SEQUENCE LISTING (410 sequences)

SEQ ID NO: 1 - protein, 246 aa, synthetic construct
GSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLGKPIPNPLLGLDSTGLNDIFEAQKIEWHEHHHHHHHH

SEQ ID NO: 2 - protein, 232 aa, synthetic construct
GSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLGLNDIFEAQKIEWHEHHHHHHHH

SEQ ID NO: 3 - protein, 450 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLS

SEQ ID NO: 4 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 125 aa, synthetic construct
EVQLVESGGDLVKPGGSLRLSCVASGFTFRDYAMTWVRQAPGKGLQWVSSISGSGGNTYYADAVKGRFTISRDNAKNTLYLQMNSLRAEDTAVYYCAKDRLSITIRPRYYGLDVWGQGTLVTVSS

SEQ ID NO: 6 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEANDTGVYYCMQALQTPYTFGQGTKLEIK

SEQ ID NO: 7 - protein, 459 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYYGLWVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 8 - protein, 222 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEANDTGVYYCMQGLQSPLTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 9 - protein, 459 aa, synthetic construct
EVQLVESGGDLVKPGGSLRLSCVASGFTFRDYAMDWVRQAPGKGLQWLSLISGSGGKTYYADAVKGRFTISRDNAKNTLYLQMNSLRAEDTAVYYCAVDRLTKTIRPQYYGLWVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 10 - protein, 222 aa, synthetic construct
DIVMTQTPLSLSVSPGETASISCRSSQSLLYSIGYNYLDWYMQKPGQSPQGLIYLGSKRASGVSDRFSGSGSGADFTLKISRVEADDAGVYYCMQGLQSPLTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 11 - protein, 459 aa, synthetic construct
EVQLVESGGDLVKPGGSLRLSCVASGFTFRDYAMDWVRQAPGKGLQWLSLISGSGGKTYYADAVKGRFTISRDNAKNTLYLQMNSLRAEDTAVYYCAVDRLTKTIRPQYYGLWSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 12 - protein, 459 aa, synthetic construct
EVQLVESGGDLVKPGGSLRLSCVASGFPFRDYAMDWVRQAPGKGLQWLSLISGSGGKTYYADAVKGRFTISRDNAKNTLYLQMNSLRAEDTAVYYCAVDRLTKTIRPQYYGLWVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 13 - protein, 459 aa, synthetic construct
EVQLVESGGDLVKPGGSLRLSCVASGLPFRDYAMAWVRQAPGKGLQWLSLISGSGGKTYYADAVKGRFTISRDNAKNTLYLQMNSLRAEDTAVYYCAVDRLTKTIRPQYYGLWVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 14 - protein, 459 aa, synthetic construct
EVQLVESGGDLVKPGGSLRLSCVASGFPFRDYAMDWVRQAPGKGLQWLSLISGSGGKTYYADAVKGRFTISRDNAKNTLYLQMNSLRAEDTAVYYCAVDRLTKTIRPQYYGLWSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 15 - protein, 459 aa, synthetic construct
EVQLVESGGDLVKPGGSLRLSCVASGFPFRDYAMTWVRQAPGKGLQWLSLISGSGGKTYYADAVKGRFTISRDNAKNTLYLQMNSLRAEDTAVYYCAVDRLTKTIRPQYYGLWHWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 16 - protein, 459 aa, synthetic construct
EVQLVESGGDLVKPGGSLRLSCVASGFTFRDYAMTWVRQAPGKGLQWLSLISGSGGKTYYADAVKGRFTISRDNAKNTLYLQMNSLRAEDTAVYYCAVDRLTKTIRPQYYGLWHWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 17 - protein, 222 aa, synthetic construct
DIVMTQTPLSLSVSPGETASISCRSSQSLTYSIGYNYLDWYMQKPGQSPQGLIYLGSKRASGVSDRFSGSGSGADFTLKISRVEADDAGVYYCMQGLHSPLSFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 18 - protein, 222 aa, synthetic construct
DIVMTQTPLSLSVSPGETASISCRSSKSLLYSIGYNYLDWYMQKPGQSPQGLIYLGSKRASGVSDRFSGSGSGADFTLKISRVEADDAGVYYCMQGLHSPLTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 19 - protein, 222 aa, synthetic construct
DIVMTQTPLSLSVSPGETASISCRSSQSLTYSIGYNYLDWYMQKPGQSPQGLIYLGSKRASGVSDRFSGSGSGADFTLKISRVEADDAGVYYCMQGLHSPLGFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 20 - protein, 222 aa, synthetic construct
DIVMTQTPLSLSVSPGETASISCRSSKSLTYSIGYNYLDWYMQKPGQSPQGLIYLGSKRASGVSDRFSGSGSGADFTLKISRVEADDAGVYYCMQGLHSPLGFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 21 - protein, 222 aa, synthetic construct
DIVMTQTPLSLSVSPGETASISCRSSKSLTYSIGYNYLDWYMQKPGQSPQGLIYLGSKRASGVSDRFSGSGSGADFTLKISRVEADDAGVYYCMQGLHSPLSFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 22 - protein, 222 aa, synthetic construct
DIVMTQTPLSLSVSPGETASISCRSSQSLTYSIGYNYLDWYMQKPGQSPQGLIYLGSKRASGVSDRFSGSGSGADFTLKISRVEADDAGVYYCMQGLQSPLGFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 23 - protein, 125 aa, synthetic construct
EVQLVESGGDLVKPGGSLRLSCVASGFTFRDYAMTWVRQAPGKGLQWVSLINGSGGKTYYADAVKGRFTISRDNAKNTLYLQMNSLRAEDTAVYYCAVDRLTKTIRPQYYGLWSWGQGTLVTVSS

SEQ ID NO: 24 - protein, 125 aa, synthetic construct
EVQLVESGGDLVKPGGSLRLSCVASGFTFRDYAMTWVRQAPGKGLQWVSLINGSGGKTYYADAVKGRFTISRDNAKNTLYLQMNSLRAEDTAVYYCAVDRLTKTIRPQYYGMWSWGQGTLVTVSS

SEQ ID NO: 25 - protein, 125 aa, synthetic construct
EVQLVESGGDLVKPGGSLRLSCVASGFTFRDYAMTWVRQAPGKGLQWVSLINGTGGKTYYADAVKGRFTISRDNAKNTLYLQMNSLRAEDTAVYYCAVDRLTKTIRPQYYGLWAWGQGTLVTVSS

SEQ ID NO: 26 - protein, 125 aa, synthetic construct
EVQLVESGGDLVKPGGSLRLSCVASGFPFRDYAMTWVRQAPGKGLQWVSLVNGSGSKTYYADAVKGRFTISRDNAKNTLYLQMNSLRAEDTAVYYCAVDRLTKTIRPQYYGLWVWGQGTLVTVSS

SEQ ID NO: 27 - protein, 125 aa, synthetic construct
EVQLVESGGDLVKPGGSLRLSCVASGFTFRDYAMTWVRQAPGKGLQWVSLINGSGGKTYYADAVKGRFTISRDNAKNTLYLQMNSLRAEDTAVYYCAVDRLTKTVRPQYYGLWSWGQGTLVTVSS

SEQ ID NO: 28 - protein, 125 aa, synthetic construct
EVQLVESGGDLVKPGGSLRLSCVASGFPFRDYAMTWVRQAPGKGLQWVSLINGSGGKTYYADAVKGRFTISRDNAKNTLYLQMNSLRAEDTAVYYCAVDRLTKTIRPQYYGLWVWGQGTLVTVSS

SEQ ID NO: 29 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEAEDTGVYYCMQGLHSPLSFGQGTKLEIK

SEQ ID NO: 30 - protein, 125 aa, synthetic construct
EVQLVESGGDLVKPGGSLRLSCVASGFTFRDYAMTWVRQAPGKGLQWVSLINGNGSKTYYADAVKGRFTISRDNAKNTLYLQMNSLRAEDTAVYYCAVDRLTKTIRPQYYGLWVWGQGTLVTVSS

SEQ ID NO: 31 - protein, 112 aa, synthetic construct
DIVMTQTPLSLSVSPGETASISCRSSKSLLYSIGYNYLDWYLQKPGQSPQGLIYLGSKRASGVPDRFSGSGSGTDFTLRISRVEADDTGVYYCMQGLHSPLTFGQGTKLEIK

SEQ ID NO: 32 - protein, 112 aa, synthetic construct
DIVMTQTPLSLSVSPGETASISCRSSQSLLYSIGYNYLDWYLQKPGQSPQGLIYLGSKRASGVPDRFSGSGNGTDFTLRISRVEAEDTGVYYCMQGLHSPLSFGQGTKLEIK

SEQ ID NO: 33 - protein, 125 aa, synthetic construct
EVQLVESGGDLVKPGGSLRLSCVASGFTFRDYAMTWVRQAPGKGLQWVSLINGNGGKTYYADAVKGRFTISRDNAKNTLYLQMNSLRAEDTAVYYCAVDRLTKTIRPQYYGLWVWGQGTLVTVSS

SEQ ID NO: 34 - protein, 125 aa, synthetic construct; identical to SEQ ID NO: 33

SEQ ID NO: 35 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEANDTGVYYCMQGLHSPLSFGQGTKLEIK

SEQ ID NO: 36 - protein, 125 aa, synthetic construct; identical to SEQ ID NO: 33

SEQ ID NO: 37 - protein, 125 aa, synthetic construct
EVQLVESGGDLVKPGGSLRLSCVASGFTFRDYAMTWVRQAPGKGLQWVSLINGSGGKTYYADAVKGRFTISRDNAKNTLYLQMNSLRAEDTAVYYCAVDRLTKTIRPQYYGLWVWGQGTLVTVSS

SEQ ID NO: 38 - protein, 125 aa, synthetic construct; identical to SEQ ID NO: 33

SEQ ID NO: 39 - protein, 125 aa, synthetic construct
EVQLVESGGDLVKPGGSLRLSCVASGFTFRDYAMTWVRQAPGKGLQWVSLINGNSGKTYYADAVKGRFTISRDNAKNTLYLQMNSLRAEDTAVYYCAVDRLTKTIRPQYYGLWVWGQGTLVTVSS

SEQ ID NO: 40 - protein, 125 aa, synthetic construct; identical to SEQ ID NO: 30

SEQ ID NO: 41 - protein, 125 aa, synthetic construct
EVQLVESGGDLVKPGGSLRLSCVASGFTFRDYAMTWVRQAPGKGLQWVSLINGDASKTYYADAVKGRFTISRDNAKNTLYLQMNSLRAEDTAVYYCAVDRLTKTIRPQYYGLWVWGQGTLVTVSS

SEQ ID NO: 42 - protein, 125 aa, synthetic construct; identical to SEQ ID NO: 37

SEQ ID NO: 43 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEADDTGVYYCMQGLHSPLTFGQGTKLEIK

SEQ ID NO: 44 - protein, 125 aa, synthetic construct
EVQLVESGGDLVKPGGSLRLSCVASGFTFRDYAMTWVRQAPGKGLQWVSLINGSGTKTYYADAVKGRFTISRDNAKNTLYLQMNSLRAEDTAVYYCAVDRLTKTIRPQYYGLWVWGQGTLVTVSS

SEQ ID NO: 45 - protein, 125 aa, synthetic construct; identical to SEQ ID NO: 37

SEQ ID NO: 46 - protein, 125 aa, synthetic construct; identical to SEQ ID NO: 33

SEQ ID NO: 47 - protein, 125 aa, synthetic construct; identical to SEQ ID NO: 37

SEQ ID NO: 48 - protein, 125 aa, synthetic construct; identical to SEQ ID NO: 37

SEQ ID NO: 49 - protein, 125 aa, synthetic construct; identical to SEQ ID NO: 37

SEQ ID NO: 50 - protein, 125 aa, synthetic construct; identical to SEQ ID NO: 37

SEQ ID NO: 51 - protein, 125 aa, synthetic construct; identical to SEQ ID NO: 37

SEQ ID NO: 52 - protein, 125 aa, synthetic construct; identical to SEQ ID NO: 33

SEQ ID NO: 53 - protein, 125 aa, synthetic construct; identical to SEQ ID NO: 37

SEQ ID NO: 54 - protein, 125 aa, synthetic construct
EVQLVESGGDLVKPGGSLRLSCVASGLPFRDYAMAWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 55 - protein, 125 aa, synthetic construct; identical to SEQ ID NO: 37

SEQ ID NO: 56 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEAEDTGVYYCMQGLHSPLTFGQGTKLEIK

SEQ ID NO: 57 - protein, 125 aa, synthetic construct; identical to SEQ ID NO: 37

SEQ ID NO: 58 - protein, 125 aa, synthetic construct; identical to SEQ ID NO: 37

SEQ ID NO: 59 - protein, 125 aa, synthetic construct
EVQLVESGGDLVKPGGSLRLSCVASGFTFRDYAMTWVRQAPGKGLQWVSLINGDGSKVYYADAVKGRFTISRDNAKNTLYLQMNSLRAEDTAVYYCAVDRLTKTIRPQYYGLWVWGQGTLVTVSS

SEQ ID NO: 60 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEAEDTGVYYCMQGLRSPLSFGQGTKLEIK

SEQ ID NO: 61 - protein, 112 aa, synthetic construct; identical to SEQ ID NO: 56

SEQ ID NO: 62 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEANDTGVYYCMQGLHSPLTFGQGTKLEIK

SEQ ID NO: 63 - protein, 125 aa, synthetic construct; identical to SEQ ID NO: 37

SEQ ID NO: 64 - protein, 125 aa, synthetic construct; identical to SEQ ID NO: 37

SEQ ID NO: 65 - protein, 125 aa, synthetic construct; identical to SEQ ID NO: 37

SEQ ID NO: 66 - protein, 125 aa, synthetic construct; identical to SEQ ID NO: 37

SEQ ID NO: 67 - protein, 125 aa, synthetic construct; identical to SEQ ID NO: 37

SEQ ID NO: 68 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEAEDTGVYYCMQGLQSPLSFGQGTKLEIK

SEQ ID NO: 69 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYYGLWSWGQGTLVTVSS

SEQ ID NO: 70 - protein, 112 aa, synthetic construct
DIVMTQTPLSLSVSPGETASISCRSSQSLLYSIGYNYLDWYLQKPGQSPQGLIYLGSKRASGVPDRFSGSGSGTDFTLRISRVEADDTGVYYCMQGLQSPLSFGQGTKLEIK

SEQ ID NO: 71 - protein, 112 aa, synthetic construct; identical to SEQ ID NO: 68

SEQ ID NO: 72 - protein, 125 aa, synthetic construct; identical to SEQ ID NO: 33

SEQ ID NO: 73 - protein, 125 aa, synthetic construct
EVQLVESGGDLVKPGGSLRLSCVASGFPFRDFAMTWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 74 - protein, 125 aa, synthetic construct
EVQLVESGGDLVKPGGSLRLSCVASGLPFRDYAMTWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 75 - protein, 125 aa, synthetic construct
EVQLVESGGDLVKPGGSLRLSCVASGFTFRDYAMTWVRQAPGKGLQWVSLINGDGSKTYYADAVKGRFTISRDNAKNTLYLQMNSLRAEDTAVYYCAVDRLTKTIRPQYYGLWVWGQGTLVTVSS

SEQ ID NO: 76 - protein, 459 aa, synthetic construct
EVQLVESGGDLVKPGGSLRLSCVASGFTFRDYAMTWVRQAPGKGLQWVSTITGSGGNTYYADAVKGRFTISRDNAKNTLYLQMNSLRAEDTAVYYCAKDRLSITIRPRYYGLDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 77 - protein, 222 aa, synthetic construct
DIVMTQTPLSLSVSPGETASISCRASQSLLYSIGYNYLDWYLQKPGQSPQGLIYLGSKRASGVPDRFSGSGSGTDFTLRISRVEANDTGVYYCMQAIQAPYTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 78 - protein, 459 aa, synthetic construct
EVQLVESGGDLVKPGGSLRLSCVASGFTFRDYAMTWVRQAPGKGLQWVSTISGSGGKTYYADAVKGRFTISRDNAKNTLYLQMNSLRAEDTAVYYCAVDRLTKTIRPQYYGLSVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 79 - protein, 222 aa, synthetic construct
DIVMTQTPLSLSVSPGETASISCRASQSLLYSIGYNYLDWYLQKPGQSPQGLIYLGSKRASGVPDRFSGSGSGTDFTLRISRVEANDTGVYYCMQALRDPYTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 80 - protein, 5 aa, synthetic construct
DYAMT

SEQ ID NO: 81 - protein, 7 aa, synthetic construct
GFTFRDY

SEQ ID NO: 82 - protein, 8 aa, synthetic construct
GFTFRDYA

SEQ ID NO: 83 - protein, 17 aa, synthetic construct
LISGSGGKTYYADAVKG

SEQ ID NO: 84 - protein, 6 aa, synthetic construct
SGSGGK

SEQ ID NO: 85 - protein, 8 aa, synthetic construct
ISGSGGKT

SEQ ID NO: 86 - protein, 16 aa, synthetic construct
DRLTKTIRPQYYGLWV

SEQ ID NO: 87 - protein, 18 aa, synthetic construct
AVDRLTKTIRPQYYGLWV

SEQ ID NO: 88 - protein, 16 aa, synthetic construct
RSSQSLLYSIGYNYLD

SEQ ID NO: 89 - protein, 11 aa, synthetic construct
QSLLYSIGYNY

SEQ ID NO: 90 - protein, 7 aa, synthetic construct
LGSKRAS

SEQ ID NO: 91 - protein, 9 aa, synthetic construct
MQGLQTPLT

SEQ ID NO: 92 - protein, 9 aa, synthetic construct
FTFRDYAMT

SEQ ID NO: 93 - protein, 17 aa, synthetic construct
SISGSGGNTYYADAVKG

SEQ ID NO: 94 - protein, 17 aa, synthetic construct
KDRLSITIRPRYYGLDV

SEQ ID NO: 95 - protein, 17 aa, synthetic construct
TITGSGGNTYYADAVKG

SEQ ID NO: 96 - protein, 17 aa, synthetic construct
TISGSGGKTYYADAVKG

SEQ ID NO: 97 - protein, 17 aa, synthetic construct
VDRLTKTIRPQYYGLWV

SEQ ID NO: 98 - protein, 17 aa, synthetic construct
VDRLTKTIRPQYYGLSV

SEQ ID NO: 99 - protein, 16 aa, synthetic construct
RASQSLLYSIGYNYLD

SEQ ID NO: 100 - protein, 9 aa, synthetic construct
MQALQTPYT

SEQ ID NO: 101 - protein, 9 aa, synthetic construct
MQGLQSPLT

SEQ ID NO: 102 - protein, 9 aa, synthetic construct
MQAIQAPYT

SEQ ID NO: 103 - protein, 9 aa, synthetic construct
MQALRDPYT

SEQ ID NO: 104 - protein, 10 aa, synthetic construct
GFTFRDYAMT

SEQ ID NO: 105 - protein, 16 aa, synthetic construct
RADQSLLYSIGYNYLD

SEQ ID NO: 106 - protein, 10 aa, synthetic construct
GFNFRDYAMT

SEQ ID NO: 107 - protein, 10 aa, synthetic construct
GFPFRDYAMT

SEQ ID NO: 108 - protein, 17 aa, synthetic construct
LINGSGGKTYYADAVKG

SEQ ID NO: 109 - protein, 17 aa, synthetic construct
MISGSGGKTYYADAVKG

SEQ ID NO: 110 - protein, 17 aa, synthetic construct
FISGSGGKTYYADAVKG

SEQ ID NO: 111 - protein, 16 aa, synthetic construct
DRLTKTIRPQYYGMWV

SEQ ID NO: 112 - protein, 16 aa, synthetic construct
DRLTKTIRPQYYGLWH

SEQ ID NO: 113 - protein, 16 aa, synthetic construct
DRKTKTIRPQYYGLWV

SEQ ID NO: 114 - protein, 16 aa, synthetic construct
DRLTRTIRPQYYGLWV

SEQ ID NO: 115 - protein, 16 aa, synthetic construct
DRLTKTIRPQYYGLWG

SEQ ID NO: 116 - protein, 16 aa, synthetic construct
DRLTKTIRPQYYGLWQ

SEQ ID NO: 117 - protein, 16 aa, synthetic construct
DRMTKTIRPQYYGLWV

SEQ ID NO: 118 - protein, 16 aa, synthetic construct
DRLTKTIRPQYYGLWK

SEQ ID NO: 119 - protein, 16 aa, synthetic construct
DRLTITIRPQYYGLWV

SEQ ID NO: 120 - protein, 16 aa, synthetic construct
DRLTKTIRPQYLGLWV

SEQ ID NO: 121 - protein, 16 aa, synthetic construct
DRLTKTIRPQYRGLWV

SEQ ID NO: 122 - protein, 16 aa, synthetic construct
RSSQSTLYSIGYNYLD

SEQ ID NO: 123 - protein, 16 aa, synthetic construct
RSSESLLYSIGYNYLD

SEQ ID NO: 124 - protein, 16 aa, synthetic construct
RSSQSLLYSIGMNYLD

SEQ ID NO: 125 - protein, 16 aa, synthetic construct
RSSQSLLYSIGLNYLD

SEQ ID NO: 126 - protein, 16 aa, synthetic construct
RSSHSLLYSIGYNYLD

SEQ ID NO: 127 - protein, 16 aa, synthetic construct
RSSLSLLYSIGYNYLD

SEQ ID NO: 128 - protein, 16 aa, synthetic construct
RSSQSLLYSVGYNYLD

SEQ ID NO: 129 - protein, 16 aa, synthetic construct
RSSTSLLYSIGYNYLD

SEQ ID NO: 130 - protein, 16 aa, synthetic construct
RSSQSLLYSPGYNYLD

SEQ ID NO: 131 - protein, 16 aa, synthetic construct
RSSQSLLYSAGYNYLD

SEQ ID NO: 132 - protein, 16 aa, synthetic construct
RSSQSLAYSIGYNYLD

SEQ ID NO: 133 - protein, 7 aa, synthetic construct
MGSKRAS

SEQ ID NO: 134 - protein, 7 aa, synthetic construct
LGHKRAS

SEQ ID NO: 135 - protein, 7 aa, synthetic construct
LGNKRAS

SEQ ID NO: 136 - protein, 7 aa, synthetic construct
LGSKRMS

SEQ ID NO: 137 - protein, 7 aa, synthetic construct
LGTKRAS

SEQ ID NO: 138 - protein, 9 aa, synthetic construct
MQPLQSPLT

SEQ ID NO: 139 - protein, 9 aa, synthetic construct
MQGLQSPLG

SEQ ID NO: 140 - protein, 9 aa, synthetic construct
MNGLQSPLT

SEQ ID NO: 141 - protein, 9 aa, synthetic construct
MQGLQSPLS

SEQ ID NO: 142 - protein, 9 aa, synthetic construct
MQGLQSPLN

SEQ ID NO: 143 - protein, 9 aa, synthetic construct
MQGLQSPLL

SEQ ID NO: 144 - protein, 9 aa, synthetic construct
MQGLQSPNT

SEQ ID NO: 145 - protein, 19 aa, synthetic construct
RSSQSLLYSIGYNYLDWYL

SEQ ID NO: 146 - protein, 9 aa, synthetic construct
FAFDDYAMT

SEQ ID NO: 147 - protein, 9 aa, synthetic construct
FAFGDYAMT

SEQ ID NO: 148 - protein, 9 aa, synthetic construct
FAFRDYAMT

SEQ ID NO: 149 - protein, 9 aa, synthetic construct
FAFRDYAMV

SEQ ID NO: 150 - protein, 9 aa, synthetic construct
FDFGDYAMT

SEQ ID NO: 151 - protein, 9 aa, synthetic construct
FDFRDYAMT

SEQ ID NO: 152 - protein, 9 aa, synthetic construct
FDFRDYAMV

SEQ ID NO: 153 - protein, 9 aa, synthetic construct
FIFRDYAMT

SEQ ID NO: 154 - protein, 9 aa, synthetic construct
FPFGDYAMT

SEQ ID NO: 155 - protein, 9 aa, synthetic construct
FPFNDFAMT

SEQ ID NO: 156 - protein, 9 aa, synthetic construct
FPFRDFAMT

SEQ ID NO: 157 - protein, 9 aa, synthetic construct
FPFRDYAMS

SEQ ID NO: 158 - protein, 9 aa, synthetic construct
FPFRDYAMT

SEQ ID NO: 159 - protein, 9 aa, synthetic construct
FPFRDYAMV

SEQ ID NO: 160 - protein, 9 aa, synthetic construct
FSFNDYAMV

SEQ ID NO: 161 - protein, 9 aa, synthetic construct
FSFRDYAMT

SEQ ID NO: 162 - protein, 9 aa, synthetic construct
FTFGDYAMT

SEQ ID NO: 163 - protein, 9 aa, synthetic construct
FTFSDYAMT

SEQ ID NO: 164 - protein, 9 aa, synthetic construct
FTFSDYAMV

SEQ ID NO: 165 - protein, 9 aa, synthetic construct
IAFRDYAMT

SEQ ID NO: 166 - protein, 9 aa, synthetic construct
IAFRDYAMV

SEQ ID NO: 167 - protein, 9 aa, synthetic construct
IAFSDYAMV

SEQ ID NO: 168 - protein, 9 aa, synthetic construct
IDFRDYAMV

SEQ ID NO: 169 - protein, 9 aa, synthetic construct
IPFGDYAMA

SEQ ID NO: 170 - protein, 9 aa, synthetic construct
IPFGDYAMT

SEQ ID NO: 171 - protein, 9 aa, synthetic construct
IPFGDYAMV

SEQ ID NO: 172 - protein, 9 aa, synthetic construct
IRFGDYAMT

SEQ ID NO: 173 - protein, 9 aa, synthetic construct
IRFRDYAMV

SEQ ID NO: 174 - protein, 9 aa, synthetic construct
ISFGDYAMA

SEQ ID NO: 175 - protein, 9 aa, synthetic construct
ISFRDYAMV

SEQ ID NO: 176 - protein, 9 aa, synthetic construct
ITFGDYAMT

SEQ ID NO: 177 - protein, 9 aa, synthetic construct
ITFRDYAMT

SEQ ID NO: 178 - protein, 9 aa, synthetic construct
ITFRDYAMV

SEQ ID NO: 179 - protein, 9 aa, synthetic construct
ITFSDYAMV

SEQ ID NO: 180 - protein, 9 aa, synthetic construct
ITSRDYAMV

SEQ ID NO: 181 - protein, 9 aa, synthetic construct
LAFRDYAMV

SEQ ID NO: 182 - protein, 9 aa, synthetic construct
LDFGDYAMT

SEQ ID NO: 183 - protein, 9 aa, synthetic construct
LDFRDYAMV

SEQ ID NO: 184 - protein, 9 aa, synthetic construct
LPFGDYAMT

SEQ ID NO: 185 - protein, 9 aa, synthetic construct
LPFRDYAMA

SEQ ID NO: 186 - protein, 9 aa, synthetic construct
LPFRDYAMT

SEQ ID NO: 187 - protein, 9 aa, synthetic construct
LRFGDYAMV

SEQ ID NO: 188 - protein, 9 aa, synthetic construct
LRFRDYAMT

SEQ ID NO: 189 - protein, 9 aa, synthetic construct
LSFRDYAMV

SEQ ID NO: 190 - protein, 9 aa, synthetic construct
LTFRDYAMT

SEQ ID NO: 191 - protein, 9 aa, synthetic construct
LTFSDYAMT

SEQ ID NO: 192 - protein, 9 aa, synthetic construct
LTFSDYAMV

SEQ ID NO: 193 - protein, 9 aa, synthetic construct
VAFRDYAMT

SEQ ID NO: 194 - protein, 9 aa, synthetic construct
LDFRDYAMT

SEQ ID NO: 195 - protein, 17 aa, synthetic construct
LINGDASKTYYADAVKG

SEQ ID NO: 196 - protein, 17 aa, synthetic construct
LINGDGSKTYYADAVKG

SEQ ID NO: 197 - protein, 17 aa, synthetic construct
LINGDGSKVYYADAVKG

SEQ ID NO: 198 - protein, 17 aa, synthetic construct
LINGIGGKTYYADAVKG

SEQ ID NO: 199 - protein, 17 aa, synthetic construct
LINGNGGKIYYADAVKG

SEQ ID NO: 200 - protein, 17 aa, synthetic construct
LINGNGGKTYYADAVKG

SEQ ID NO: 201 - protein, 17 aa, synthetic construct
LINGNGGRTYYADAVKG

SEQ ID NO: 202 - protein, 17 aa, synthetic construct
LINGNGSKTYYADAVKG

SEQ ID NO: 203 - protein, 17 aa, synthetic construct
LINGNSGKTYYADAVKG

SEQ ID NO: 204 - protein, 17 aa, synthetic construct
LINGNSGKVYYADAVKG

SEQ ID NO: 205 - protein, 17 aa, synthetic construct
LINGSGDKTYYADAVKG

SEQ ID NO: 206 - protein, 17 aa, synthetic construct
LINGSGDRTYYADAVKG

SEQ ID NO: 207 - protein, 17 aa, synthetic construct
LINGSGGRTYYADAVKG

SEQ ID NO: 208 - protein, 17 aa, synthetic construct
LINGSGGKTYYADAVKD

SEQ ID NO: 209 - protein, 17 aa, synthetic construct
LINGSGGKVYYADAVKG

SEQ ID NO: 210 - protein, 17 aa, synthetic construct
LINGSGNKTYYADAVKG

SEQ ID NO: 211 - protein, 17 aa, synthetic construct
LINGSGTKTYYADAVKG

SEQ ID NO: 212 - protein, 17 aa, synthetic construct
LINGTGGKTYYADAVKG

SEQ ID NO: 213 - protein, 17 aa, synthetic construct
LISGSGGKSYYADAVKG

SEQ ID NO: 214 - protein, 17 aa, synthetic construct
LISGSGGNTYYADAVKG

SEQ ID NO: 215 - protein, 17 aa, synthetic construct
LISGSGTKTYYADAVKG

SEQ ID NO: 216 - protein, 17 aa, synthetic construct
LVNGSGSKTYYADAVKG

SEQ ID NO: 217 - protein, 17 aa, synthetic construct
SINGSGGKTYYADAVKG

SEQ ID NO: 218 - protein, 17 aa, synthetic construct
SINGSGGSIYYADAVKG

SEQ ID NO: 219 - protein, 17 aa, synthetic construct
TINGNGGKTYYADAVKG

SEQ ID NO: 220 - protein, 17 aa, synthetic construct
TINGSGNKTYYADAVKG

SEQ ID NO: 221 - protein, 17 aa, synthetic construct
ADRLTKTIRPQYYGLWV

SEQ ID NO: 222 - protein, 17 aa, synthetic construct
IDRLTKTIRPQYYGLWV

SEQ ID NO: 223 - protein, 17 aa, synthetic construct
RDRLTKTIRPQYYGLQV

SEQ ID NO: 224 - protein, 17 aa, synthetic construct
SDRLTKTIRPQYMGMWV

SEQ ID NO: 225 - protein, 17 aa, synthetic construct
TDRLTHTIRPQYYGLWS

SEQ ID NO: 226 - protein, 17 aa, synthetic construct
TDRLTKTIRPQYYGLWV

SEQ ID NO: 227 - protein, 17 aa, synthetic construct
TDRLTKTIRPQYYGMWV

SEQ ID NO: 228 - protein, 17 aa, synthetic construct
VDRITKTIRPQYTGLWV

SEQ ID NO: 229 - protein, 17 aa, synthetic construct
VDRLTKMIRPQYYGLWS

SEQ ID NO: 230 - protein, 17 aa, synthetic construct
VDRLTKTIRPAYQGLWV

SEQ ID NO: 231 - protein, 17 aa, synthetic construct
VDRLTKTIRPQYAGLWS

SEQ ID NO: 232 - protein, 17 aa, synthetic construct
VDRLTKTIRPQYAGLWV

SEQ ID NO: 233 - protein, 17 aa, synthetic construct
VDRLTKTIRPQYEGLWL

SEQ ID NO: 234 - protein, 17 aa, synthetic construct
VDRLTKTIRPQYEGLWV

SEQ ID NO: 235 - protein, 17 aa, synthetic construct
VDRLTKTIRPQYFGLWV

SEQ ID NO: 236 - protein, 17 aa, synthetic construct
VDRLTKTIRPQYLGLWV

SEQ ID NO: 237 - protein, 17 aa, synthetic construct
VDRLTKTIRPQYMGLWV

SEQ ID NO: 238 - protein, 17 aa, synthetic construct
VDRLTKTIRPQYNGLWV

SEQ ID NO: 239 - protein, 17 aa, synthetic construct
VDRLTKTIRPQYQGLWV

SEQ ID NO: 240 - protein, 17 aa, synthetic construct
VDRLTKTIRPQYRGFWD

SEQ ID NO: 241 - protein, 17 aa, synthetic construct
VDRLTKTIRPQYSGLWD

SEQ ID NO: 242 - protein, 17 aa, synthetic construct
VDRLTKTIRPQYSGLWV

SEQ ID NO: 243 - protein, 17 aa, synthetic construct
VDRLTKTIRPQYTGLWV

SEQ ID NO: 244 - protein, 17 aa, synthetic construct
VDRLTKTIRPQYYGLWA

SEQ ID NO: 245 - protein, 17 aa, synthetic construct
VDRLTKTIRPQYYGLWD

SEQ ID NO: 246 - protein, 17 aa, synthetic construct
VDRLTKTIRPQYYGLWF

SEQ ID NO: 247 - protein, 17 aa, synthetic construct
VDRLTKTIRPQYYGLWH

SEQ ID NO: 248 - protein, 17 aa, synthetic construct
VDRLTKTIRPQYYGLWS

SEQ ID NO: 249 - protein, 17 aa, synthetic construct
VDRLTKTIRPQYYGMWH

SEQ ID NO: 250 - protein, 17 aa, synthetic construct
VDRLTKTIRPQYYGMWS

SEQ ID NO: 251 - protein, 17 aa, synthetic construct
VDRLTKTIRPVYRGLWV

SEQ ID NO: 252 - protein, 17 aa, synthetic construct
VDRLTKTVRPQYYGLWS

SEQ ID NO: 253 - protein, 17 aa, synthetic construct
VDRLTKTVRPQYYGLWV

SEQ ID NO: 254 - protein, 17 aa, synthetic construct
VDRLTNTIRPQYMGLWV

SEQ ID NO: 255 - protein, 17 aa, synthetic construct
VDRMTKTIRPQYYGLWD

SEQ ID NO: 256 - protein, 17 aa, synthetic construct
VDRMTKTIRPQYYGLWS

SEQ ID NO: 257 - protein, 17 aa, synthetic construct
VDRMTKTIRPQYYGLWV

SEQ ID NO: 258 - protein, 16 aa, synthetic construct
RSSGSVLYSSGYNYLD

SEQ ID NO: 259 - protein, 16 aa, synthetic construct
RSSKSLIYSIGYNYVD

SEQ ID NO: 260 - protein, 16 aa, synthetic construct
RSSKSLLYSGGYNYLD

SEQ ID NO: 261 - protein, 16 aa, synthetic construct
RSSKSLLYSIGLNYVD

SEQ ID NO: 262 - protein, 16 aa, synthetic construct
RSSKSLLYSIGYNYID

SEQ ID NO: 263 - protein, 16 aa, synthetic construct
RSSKSLLYSIGYNYLD

SEQ ID NO: 264 - protein, 16 aa, synthetic construct
RSSKSLLYSIGYNYVD

SEQ ID NO: 265 - protein, 16 aa, synthetic construct
RSSKSLLYSTGRNYLD

SEQ ID NO: 266 - protein, 16 aa, synthetic construct
RSSKSLLYSTGYNYLD

SEQ ID NO: 267 - protein, 16 aa, synthetic construct
RSSKSLLYSVGYNYLD

SEQ ID NO: 268 - protein, 16 aa, synthetic construct
RSSKSLLYSVGYNYVD

SEQ ID NO: 269 - protein, 16 aa, synthetic construct
RSSKSLSYSIGYNYVD

SEQ ID NO: 270 - protein, 16 aa, synthetic construct
RSSKSVLYSAGYNYVD

SEQ ID NO: 271 - protein, 16 aa, synthetic construct
RSSQSLIYSNRYNYLD

SEQ ID NO: 272 - protein, 16 aa, synthetic construct
RSSQSLLYSAGTNYLD

SEQ ID NO: 273 - protein, 16 aa, synthetic construct
RSSQSLLYSAGYNYID

SEQ ID NO: 274 - protein, 16 aa, synthetic construct
RSSQSLLYSAGYNYVD

SEQ ID NO: 275 - protein, 16 aa, synthetic construct
RSSQSLLYSGDYNYLD

SEQ ID NO: 276 - protein, 16 aa, synthetic construct
RSSQSLLYSGGANYLD

SEQ ID NO: 277 - protein, 16 aa, synthetic construct
RSSQSLLYSGLYNYLD

SEQ ID NO: 278 - protein, 16 aa, synthetic construct
RSSQSLLYSIGANYLD

SEQ ID NO: 279 - protein, 16 aa, synthetic construct
RSSQSLLYSIGFNYID

SEQ ID NO: 280 - protein, 16 aa, synthetic construct
RSSQSLLYSIGFNYVD

SEQ ID NO: 281 - protein, 16 aa, synthetic construct
RSSQSLLYSIGNNYID

SEQ ID NO: 282 - protein, 16 aa, synthetic construct
RSSQSLLYSIGSNYLD

SEQ ID NO: 283 - protein, 16 aa, synthetic construct
RSSQSLLYSIGSNYVD

SEQ ID NO: 284 - protein, 16 aa, synthetic construct
RSSQSLLYSIGYNYID

SEQ ID NO: 285 - protein, 16 aa, synthetic construct
RSSQSLLYSIGYNYVD

SEQ ID NO: 286 - protein, 16 aa, synthetic construct
RSSQSLLYSNRTNYLD

SEQ ID NO: 287 - protein, 16 aa, synthetic construct
RSSQSLLYSSGINYLD

SEQ ID NO: 288 - protein, 16 aa, synthetic construct
RSSQSLLYSSGYNYLD

SEQ ID NO: 289 - protein, 16 aa, synthetic construct
RSSQSLLYSSGYNYVD

SEQ ID NO: 290 - protein, 16 aa, synthetic construct
RSSQSLLYSVGYNYID

SEQ ID NO: 291 - protein, 16 aa, synthetic construct
RSSQSLSYSIGYNYID

SEQ ID NO: 292 - protein, 16 aa, synthetic construct
RSSQSLSYSIGYNYLD

SEQ ID NO: 293 - protein, 16 aa, synthetic construct
RSSQSLSYSIGYNYVD

SEQ ID NO: 294 - protein, 16 aa, synthetic construct
RSSQSVLYSAGYNYLD

SEQ ID NO: 295 - protein, 16 aa, synthetic construct
RSSQSVLYSGGYNYLD

SEQ ID NO: 296 - protein, 16 aa, synthetic construct
RSSQSVLYSTGYNYVD

SEQ ID NO: 297 - protein, 16 aa, synthetic construct
RSSRSLLYSVGYNYLD

SEQ ID NO: 298 - protein, 16 aa, synthetic construct
RSSTSLLYGIGYNYLD

SEQ ID NO: 299 - protein, 16 aa, synthetic construct
RSSTSLLYSIGYNYVD

SEQ ID NO: 300 - protein, 16 aa, synthetic construct
RSSTSLQYSIGKNYLD

SEQ ID NO: 301 - protein, 16 aa, synthetic construct
RSSTSLSYSIGRNYLD

SEQ ID NO: 302 - protein, 16 aa, synthetic construct
RSSTTLLYSAGYNYVD

SEQ ID NO: 303 - protein, 7 aa, synthetic construct
AGNKRQS

SEQ ID NO: 304 - protein, 7 aa, synthetic construct
HGNKRQS

SEQ ID NO: 305 - protein, 7 aa, synthetic construct
LGDKRAA

SEQ ID NO: 306 - protein, 7 aa, synthetic construct
LGNKRAA

SEQ ID NO: 307 - protein, 7 aa, synthetic construct
LGNKRAP

SEQ ID NO: 308 - protein, 7 aa, synthetic construct
LGNKRAT

SEQ ID NO: 309 - protein, 7 aa, synthetic construct
LGNKRQA

SEQ ID NO: 310 - protein, 7 aa, synthetic construct
LGNKRQS

SEQ ID NO: 311 - protein, 7 aa, synthetic construct
LGNKRSS

SEQ ID NO: 312 - protein, 7 aa, synthetic construct
LGNRRAA

SEQ ID NO: 313 - protein, 7 aa, synthetic construct
LGNRRAS

SEQ ID NO: 314 - protein, 7 aa, synthetic construct
LGSKLAA

SEQ ID NO: 315 - protein, 7 aa, synthetic construct
LGSKLAS

SEQ ID NO: 316 - protein, 7 aa, synthetic construct
LGSKRAA

SEQ ID NO: 317 - protein, 7 aa, synthetic construct
LGSKRAG

SEQ ID NO: 318 - protein, 7 aa, synthetic construct
LGSKRAP

SEQ ID NO: 319 - protein, 7 aa, synthetic construct
LGSKRAQ

SEQ ID NO: 320 - protein, 7 aa, synthetic construct
LGSKRAR

SEQ ID NO: 321 - protein, 7 aa, synthetic construct
LGSKRAT

SEQ ID NO: 322 - protein, 7 aa, synthetic construct
LGSKRQA

SEQ ID NO: 323 - protein, 7 aa, synthetic construct
LGSKRQS

SEQ ID NO: 324 - protein, 7 aa, synthetic construct
LGSKRQT

SEQ ID NO: 325 - protein, 7 aa, synthetic construct
LGSKRSA

SEQ ID NO: 326 - protein, 7 aa, synthetic construct
LGSKRYS

SEQ ID NO: 327 - protein, 7 aa, synthetic construct
LGSNRAS

SEQ ID NO: 328 - protein, 7 aa, synthetic construct
LGTKRQA

SEQ ID NO: 329 - protein, 7 aa, synthetic construct
LGYKRAP

SEQ ID NO: 330 - protein, 7 aa, synthetic construct
LGYKRAS

SEQ ID NO: 331 - protein, 7 aa, synthetic construct
LGYKRAT

SEQ ID NO: 332 - protein, 7 aa, synthetic construct
LSNKRSA

SEQ ID NO: 333 - protein, 7 aa, synthetic construct
NGYKRAA

SEQ ID NO: 334 - protein, 7 aa, synthetic construct
QGRKRAA

SEQ ID NO: 335 - protein, 9 aa, synthetic construct
MQGLFSPPT

SEQ ID NO: 336 - protein, 9 aa, synthetic construct
MQGLHNPLT

SEQ ID NO: 337 - protein, 9 aa, synthetic construct
MQGLHNPPT

SEQ ID NO: 338 - protein, 9 aa, synthetic construct
MQGLHSPLS

SEQ ID NO: 339 - protein, 9 aa, synthetic construct
MQGLHSPLT

SEQ ID NO: 340 - protein, 9 aa, synthetic construct
MQGLHSPLV

SEQ ID NO: 341 - protein, 9 aa, synthetic construct
MQGLISPLS

SEQ ID NO: 342 - protein, 9 aa, synthetic construct
MQGLISPLT

SEQ ID NO: 343 - protein, 9 aa, synthetic construct
MQGLQSPVT

SEQ ID NO: 344 - protein, 9 aa, synthetic construct
MQGLQSPYT

SEQ ID NO: 345 - protein, 9 aa, synthetic construct
MQGLRSPLS

SEQ ID NO: 346 - protein, 9 aa, synthetic construct
MQGLRSPLT

SEQ ID NO: 347 - protein, 9 aa, synthetic construct
MQGLRSPVT

SEQ ID NO: 348 - protein, 9 aa, synthetic construct
MQGLSSPLT

SEQ ID NO: 349 - protein, 9 aa, synthetic construct
MQGLTSPLT

SEQ ID NO: 350 - protein, 9 aa, synthetic construct
MQNLHFPPT

SEQ ID NO: 351 - protein, 9 aa, synthetic construct
MQSLQSPIT

SEQ ID NO: 352 - protein, 9 aa, synthetic construct
MQSLQSPLS

SEQ ID NO: 353 - protein, 9 aa, synthetic construct
MQSLQSPLT

SEQ ID NO: 354 - protein, 9 aa, synthetic construct
MQSLQSPYT

SEQ ID NO: 355 - protein, 9 aa, synthetic construct
MQGLQSPIT

SEQ ID NO: 356 - protein, 9 aa, synthetic construct
MQGLTSPIV

SEQ ID NO: 357 - protein, 9 aa, synthetic construct
MQGLQSPLV

SEQ ID NO: 358 - protein, 10 aa, synthetic construct
GFTFRDYAMD

SEQ ID NO: 359 - protein, 10 aa, synthetic construct
GFPFRDYAMD

SEQ ID NO: 360 - protein, 10 aa, synthetic construct
GLPFRDYAMA

SEQ ID NO: 361 - protein, 18 aa, synthetic construct
AVDRLTKTIRPQYYGLWS

SEQ ID NO: 362 - protein, 18 aa, synthetic construct
AVDRLTKTIRPQYYGLWH

SEQ ID NO: 363 - protein, 16 aa, synthetic construct
RSSQSLTYSIGYNYLD

SEQ ID NO: 364 - protein, 16 aa, synthetic construct
RSSKSLTYSIGYNYLD

SEQ ID NO: 365 - protein, 9 aa, synthetic construct
MQGLHSPLG

SEQ ID NO: 366 - protein, 125 aa, synthetic construct
EVQLVESGGDLVKPGGSLRLSCVASGIPFGDYAMAWVRQAPGKGLQWVSLINGSGGKTYYADAVKGRFTISRDNAKNTLYLQMNSLRAEDTAVYYCAVDRLTKTIRPQYYGLWVWGQGTLVTVSS

SEQ ID NO: 367 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYYGMWHWGQGTLVTVSS

SEQ ID NO: 368 - protein, 125 aa, synthetic construct
EVQLVESGGDLVKPGGSLRLSCVASGFTFRDYAMTWVRQAPGKGLQWVSLINGSGNKTYYADAVKGRFTISRDNAKNTLYLQMNSLRAEDTAVYYCAVDRLTKTIRPQYYGLWVWGQGTLVTVSS

SEQ ID NO: 369 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRMTKTIRPQYYGLWDWGQGTLVTVSS

SEQ ID NO: 370 - protein, 125 aa, synthetic construct
EVQLVESGGDLVKPGGSLRLSCVASGLAFRDYAMVWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 371 - protein, 125 aa, synthetic construct
EVQLVESGGDLVKPGGSLRLSCVASGFTFRDYAMTWVRQAPGKGLQWVGLINGSGGKTYYADAVKGRFTISRDNAKNTLYLQMNSLRAEDTAVYYCAVDRLTKTIRPQYYGLWVWGQGTLVTVSS

SEQ ID NO: 372 - protein, 125 aa, synthetic construct
EVQLVESGGDLVKPGGSLRLSCVASGLTFRDYAMTWVRQAPGKGLQWVGLISGSGGKTYYADAVKGRFTISRDNAKNTLYLQMNSLRAEDTAVYYCAVDRLTKTIRPQYYGLWVWGQGTLVTVSS

SEQ ID NO: 373 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYYGLWFWGQGTLVTVSS

SEQ ID NO: 374 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYYGLWAWGQGTLVTVSS

SEQ ID NO: 375 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCASDRLTKTIRPQYMGMWVWGQGTLVTVSS

SEQ ID NO: 376 - protein, 125 aa, synthetic construct
EVQLVESGGDLVKPGGSLRLSCVASGVAFRDYAMTWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 377 - protein, 125 aa, synthetic construct
VVQLVESGGDLVKPGGSLRLSCVASGFPFRDYAMSWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 378 - protein, 125 aa, synthetic construct
EVQLVESGGDLVKPGGSLRLSCVASGFTFRDYAMTWVRQAPGKGLQWVSLINGSGDKTYYADAVKGRFTISRDNAKNTLYLQMNSLRAEDTAVYYCAVDRLTKTIRPQYYGLWVWGQGTLVTVSS

SEQ ID NO: 379 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYYGLWHWGQGTLVTVSS

SEQ ID NO: 380 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYYGLWDWGQGTLVTVSS

SEQ ID NO: 381 - protein, 125 aa, synthetic construct
EVQLVESGGDLVKPGGSLRLSCVASGFSFRDYAMTWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 382 - protein, 125 aa, synthetic construct
EVQLVESGGDLVKPGGSLRLSCVASGFTFRDYAMTWVRQAPGKGLQWVSLINGSGGKTYYADAVKDRFTISRDNAKNTLYLQMNSLRAEDTAVYYCAVDRLTKTIRPQYYGLWVWGQGTLVTVSS

SEQ ID NO: 383 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRMTKTIRPQYYGLWSWGQGTLVTVSS

SEQ ID NO: 384 - protein, 125 aa, synthetic construct
EVQLVESGGDLVKPGGSLRLSCVASGIRFRDYAMVWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 385 - protein, 125 aa, synthetic construct
EVQLVESGGDLVKPGGSLRLSCVASGFPFRDYAMVWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 386 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTVRPQYYGLWVWGQGTLVTVSS

SEQ ID NO: 387 - protein, 125 aa, synthetic construct
EVQLVESGGDLVKPGGSLRLSCVASGISFRDYAMVWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 388 - protein, 125 aa, synthetic construct
EVQLVESGGDLVKPGGSLRLSCVASGLSFRDYAMVWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 389 - protein, 125 aa, synthetic construct
EVQLVESGGDLVKPGGSLRLSCVASGLRFRDYAMTWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 390 - protein, 125 aa, synthetic construct
EVQLVESGGDLVKPGGSLRLSCVASGFTFRDYAMTWVRQAPGKGLQWVSLISGSGGKSYYADAVKGRFTISRDNAKNTLYLQMNSLRAEDTAVYYCAVDRLTKTIRPQYYGLWVWGQGTLVTVSS

SEQ ID NO: 391 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYYGMWSWGQGTLVTVSS

SEQ ID NO: 392 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRMTKTIRPQYYGLWVWGQGTLVTVSS

SEQ ID NO: 393 - protein, 125 aa, synthetic construct
EVQLVESGGDLVKPGGSLRLSCVASGFAFRDYAMTWVRQAPGKGLQWVSLINGSGGKTYYADAVKGRFTISRDNAKNTLYLQMNSLRAEDTAVYYCAVDRLTKTIRPQYYGLWVWGQGTLVTVSS

SEQ ID NO: 394 - protein, 125 aa, synthetic construct
EVQLVESGGDLVKPGGSLRLSCVASGLDFRDYAMTWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 395 - protein, 125 aa, synthetic construct
EVQLVESGGDLVKPGGSLRLSCVASGFTFRDYAMTWVRQAPGKGLQWVSLINGNGGRTYYADAVKGRFTISRDNAKNTLYLQMNSLRAEDTAVYYCAVDRLTKTIRPQYYGLWVWGQGTLVTVSS

SEQ ID NO: 396 - protein, 125 aa, synthetic construct
EVQLVESGGDLVKPGGSLRLSCVASGFTFRDYAMTWVRQAPGKGLQWVSLINGIGGKTYYADAVKGRFTISRDNAKNTLYLQMNSLRAEDTAVYYCAADRLTKTIRPQYYGLWVWGQGTLVTVSS

SEQ ID NO: 397 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPVYRGLWVWGQGTLVTVSS

SEQ ID NO: 398 - protein, 125 aa, synthetic construct
EVQLVESGGDLVKPGGSLRLSCVASGFTFRDYAMTWVRQAPGKGLQWVSLINGNSGKVYYADAVKGRFTISRDNAKNTLYLQMNSLRAEDTAVYYCAVDRLTKTIRPQYYGLWVWGQGTLVTVSS

SEQ ID NO: 399 - protein, 125 aa, synthetic construct
EVQLVESGGDLVKPGGSLRLSCVASGFTFRDYAMTWVRQAPGKGLQWVSLISGSGTKTYYADAVKGRFTISRDNAKNTLYLQMNSLRAEDTAVYYCAVDRLTKTIRPQYYGLWVWGQGTLVTVSS

SEQ ID NO: 400 - protein, 125 aa, synthetic construct
EVQLVESGGDLVKPGGSLRLSCVASGFIFRDYAMTWVRQAPGKGLQWVSLINGNGGKTYYADAVKGRFTISRDNAKNTLYLQMNSLRAEDTAVYYCAVDRLTKTIRPQYYGLWVWGQGTLVTVSS

SEQ ID NO: 401 - protein, 125 aa, synthetic construct
EVQLVESGGDLVKPGGSLRLSCVASGFTFRDYAMTWVRQAPGKGLQWVSLINGNGGKIYYADAVKGRFTISRDNAKNTLYLQMNSLRAEDTAVYYCAVDRLTKTIRPQYYGLWVWGQGTLVTVSS

SEQ ID NO: 402 - protein, 125 aa, synthetic construct
EVQLVESGGDLVKPGGSLRLSCVASGIAFRDYAMTWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 403 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCATDRLTHTIRPQYYGLWSWGQGTLVTVSS

SEQ ID NO: 404 - protein, 125 aa, synthetic construct
EVQLVESGGDLVKPGGSLRLSCVASGIPFRDYAMTWVRQAPGKGLQWVSLISGSGGKTYYADAVKGRFTISRDNAKNTLYLQMNSLRAEDTAVYYCAVDRLTKTIRPQYYGLWVWGQGTLVTVSS

SEQ ID NO: 405 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAVDRLTKTIRPQYRGFWDWGQGTQVTVSS

SEQ ID NO: 406 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCAIDRLTKTIRPQYYGLWVWGQGTLVTVSS

SEQ ID NO: 407 - protein, 125 aa, synthetic construct
EVQLVESGGDLVKPGGSLRLSCVASGFTFRDYAMTWVRQAPGKGLQWVSLINGNGGKTYYADAVKGRFTISRDNAKNTLYLQINSLRAEDTAVYYCAVDRLTKTIRPQYYGLWVWGQGTLVTVSS

SEQ ID NO: 408 - protein, 125 aa, synthetic construct
EVQLVESGGDLVKPGGSLRLSCVASGFTFRDYAMTWVRQAPGKGLQWVSLISGSGGKTYYADAVKGRFTISRDNAKNTLYLQMNSLRAEDTAVYYCATDRLTKTIRPQYYGMWVWGQGTLVTVSS

SEQ ID NO: 409 - protein, 9 aa, synthetic construct
IPFRDYAMT

SEQ ID NO: 410 - protein, 7 aa, synthetic construct
LGSKRLS

(19) United States
(12) Patent Application Publication
(10) Pub. No.: US 2025/0188176 A1
(43) Pub. Date: Jun. 12, 2025

(54) ANTI-IL-4RA ANTIBODIES AND USES THEREOF

(71) Applicant: INVETX, INC., Boston, MA (US)

(72) Inventors: William Brondyk, Boston, MA (US); Leila Sevigny, Boston, MA (US); Elizabeth Bosia, Boston, MA (US)

(21) Appl. No.: 18/972,367

(22) Filed: Dec. 6, 2024

Related U.S. Application Data
(60) Provisional application No. 63/606,700, filed on Dec. 6, 2023.

(51) Int. Cl. C07K 16/28; A61P 37/06; G01N 33/53; G01N 33/68

(52) U.S. Cl. CPC C07K 16/2866; A61P 37/06; G01N 33/53; G01N 33/6869; C07K 2317/565; C07K 2317/567; C07K 2317/76; C07K 2317/92; G01N 2333/7155

(57) ABSTRACT

The invention provides novel anti-IL-4RA proteins, antibodies and IL-4RA binding fragments thereof, which inhibit association of IL-4 with IL-4 receptor and are suitable for administration to a human or canine subject. The invention provides novel compositions and methods of treating, alleviating the symptoms of, or preventing, lung diseases, cardiovascular diseases, cancers, metabolic diseases, neurological diseases, and infectious diseases, comprising administering an effective amount of an anti-IL-4RA protein, antibody, or fragment thereof. The methods and compositions are used to treat or prevent IL-4-related disorders.

### INCORPORATION BY REFERENCE

This application claims priority to U.S. provisional application Ser. No. 63/606,700, filed Dec. 6, 2023, which is incorporated by reference herein in its entirety.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted via Patent Center and is hereby incorporated by reference in its entirety. Said .xml copy, created on Dec. 6, 2024 is named Y9432-99005, and is 376,327 bytes in size.

### FIELD OF THE INVENTION

The invention provides novel anti-IL-4RA proteins and antibodies that are suitable for administration to a human or canine subject. The invention also provides novel compositions and methods of treating asthma or eliciting an antiasthmatic or antiallergenic effect in a human or canine subject, comprising administering an effective amount of an anti-IL-4RA protein, antibody or fragment thereof. The methods and compositions are used to treat or prevent IL-4- and IL-13-related disorders.

### BACKGROUND OF THE INVENTION

IL-4 signals through type I and type II receptor complexes, both of which include the IL-4Rα receptor alpha chain (IL-4Rα) (CD124). The type 1 IL-4 receptor is composed of IL-4Rα chain and the common gamma chain (γC) (CD132). The γC chain is “common” in that it is a cytokine receptor sub-unit that is common to the receptor complexes for at least six different interleukin receptors: IL-2, IL-4, IL-7, IL-9, IL-15, and IL-21 receptor. The γC chain is expressed in hematopoietic cells but poorly expressed in non-hematopoietic cells. Hence, IL-4 primarily modulates the function of lymphocyte subsets, including inducing T_{H}2 polarization and IgG1/IgE class switching in B cells.

The type 2 IL-4 receptor is composed of an IL-4Rα chain and an al chain of the IL-13 receptor (IL-13Rα1) and is also referred to as the “shared” IL-4/IL-13 receptor. IL-13Rα1 is poorly expressed in lymphocytes but abundantly in all non-hematopoietic cells. IL-13 acts mainly on myeloid cells and non-hematopoietic cells, having effects on mucus production, smooth muscle contraction, and epithelium permeabilization (e.g. allergic asthma).

Interleukin 4 (IL-4) is a cytokine that induces differentiation of naive helper T cells (T_{H}0 cells) to T_{H}2 cells. Upon activation by IL-4, T_{H}2 cells subsequently produce additional IL-4 in a positive feedback loop. IL-4 is produced primarily by mast cells, T_{H}2 cells, eosinophils and basophils.

IL-4 and IL-13 cytokines are secreted glycoproteins with about 25% sequence identity. They share receptor subunits and elicit overlapping and unique biological responses.

IL-4 has many biological roles, including the stimulation of activated B cells and T cell proliferation, and the differentiation of B cells into plasma cells. It is a key regulator in humoral and adaptive immunity. IL-4 induces B cell class switching to IgE, and upregulates MHC class II production. IL-4 decreases the production of T_{H}1 cells, macrophages, IFNγ, and dendritic cells.

Overproduction of IL-4 is associated with allergies and airway inflammation observed in the lungs of patients with allergic asthma. IL-4 mediates important pro-inflammatory functions in asthma, including induction of isotype rearrangement of IgE, expression of vascular cell adhesion molecule 1 (VCAM-1), promoting eosinophilic transmigration through endothelium, mucus secretion, and T_{H}2 leading to cytokine release.

IL-4 also plays a significant effect on tumor progression. Overexpression of IL-4 receptor has been found in many types of cancer. IL-4 has been shown to drive mitogenesis, dedifferentiation, and metastasis in rhabdomyosarcoma.

IL-4RA-related diseases and conditions that affect dogs, cats, and horses are similar to those in humans, including without limitation, atopic dermatitis, asthma, chronic rhinosinusitis, eosinophilic esophagitis, eosinophilic bronchopneumopathy, eosinophilic gastroenteritis/colitis, hypereosinophilic syndrome and prugrigo nodularis.

Citation or identification of any document in this application is not an admission that such document is available as prior art to the present invention.

### SUMMARY OF THE INVENTION

In an aspect, the invention provides an antigen binding protein that specifically binds to interleukin-4 receptor alpha (IL-4RA). In certain embodiments, the IL-4RA binding protein comprises: (a) a heavy chain complementarity determining region 1 (HCDR1) comprising X₂₇X₂₈X₂₉X₃₀X₃₁X₃₂X₃₃X₃₄X₃₅, wherein X₂₇ comprises F, I, L, or V, X₂₈ comprises A, D, I, N, P, R, S, or T, X₂₉ comprises F or S, X₃₀ comprises D, G, N, R, or S, X₃₁ comprises D, X₃₂ comprises F or Y, X₃₃ comprises A, X₃₄ comprises M, and X₃₅ comprises A, S, T, or V; (b) a heavy chain complementarity determining region 2 (HCDR2) comprising X₅₀X₅₁X₅₂X₅₃X₅₄X₅₅X₅₆X₅₇X₅₈X₅₉X₆₀X₆₁X₆₂X₆₃X₆₄X₆₅X₆₆, wherein X₅₀ comprises F, L, M, S, or T, X₅₁ comprises I or V, X₅₂ comprises N, S, or T, X₅₃ comprises G, X₅₄ comprises D, I, N, S, or T, X₅₅ comprises A, G, or S, X₅₆ comprises D, G, N, S, or T, X₅₇ comprises G, K, N, R, or S, X₅₈ comprises I, S, T, or V, X₅₉ comprises Y, X₆₀ comprises Y, X₆₁ comprises A, X₆₂ comprises D, X₆₃ comprises A, X₆₄ comprises V X₆₅ comprises K, and X₆₆ comprises D or G; (c) a heavy chain complementarity determining region 3 (HCDR3) comprising X₉₈X₉₉X₁₀₀X₁₀₁X₁₀₂X₁₀₃X₁₀₄X₁₀₅X₁₀₆X₁₀₇X₁₀₈X₁₀₉X₁₁₀X₁₁₁X₁₁₂X₁₁₃X₁₁₄, wherein X₉₈ comprises A, I, K, R, S, T, or V, X₉₉ comprises D, X₁₀₀ comprises R, X₁₀₁ comprises I, K, L, or M, X₁02 comprises S or T, X₁₀₃ comprises I, H, N, K, or R, X₁₀₄ comprises M or T, X₁₀₅ comprises I or V, X₁₀₆ comprises R, X₁₀₇ comprises P, X₁₀₈ comprises A, Q, R, or V, X₁₀₉ comprises Y, X₁₁₀ comprises A, E, F, L, M, N, R, S, T, Q, or Y, X₁₁₁ comprises G, X₁₁₂ comprises F, L, or M, X₁₁₃ comprises D, Q, S, or W, and X₁₁₄ comprises, A, D, F, G, H, L, Q, R, S, or V; (d) a light chain complementarity determining region 1 (LCDR1) comprising X₂₇X₂₈X₂₉X₃₀X₃₁X₃₂X₃₃X₃₄X₃₅X₃₆X₃₇X₃₈X₃₉, wherein X₂₇ comprises E, G, H, K, L, Q, R, or T, X₂₈ comprises S or T, X₂₉ comprises L or V, X₃₀ comprises A, I, L, Q, S, or T, X₃₁ comprises Y, X₃₂ comprises G or S, X₃₃ comprises A, G, I, N, P, S, T, or V, X₃₄ comprises D, G, L, or R, X₃₅ comprises A, F, I, K, L, M, N, R, S, T, or Y, X₃₆ comprises N, X₃₇ comprises Y, X₃₈ comprises I, L, or V, and X₃₉ comprises D; (e) a light chain complementarity determining region 2 (LCDR2) comprising X₅₅X₅₆X₅₇X₅₈X₅₉X₆₀X₆₁, wherein X₅₅ comprises A, H, L, M, N, or Q, X₅₆ comprises G or S, X₅₇ comprises D, H, N, S, T, or Y, X₅₈ comprises K, N, or R, X₅₉ comprises L or R, X₆₀ comprises A, M, Q, S, or Y, and X₆₁ comprises A, G, P, Q, R, S, or T; and (f) a light chain complementarity determining region 3 (LCDR3) comprising X₉₄X₉₅X₉₆X₉₇X₉₈X₉₉X₁₀₀X₁₀₁X₁₀₉, wherein X₉₄ comprises M, X₉₅ comprises N, or Q, X₉₆ comprises A, G, N, P, or S, X₉₇ comprises I or L, X₉₈ comprises F, H, I, Q, R, S, or T, X₉₉ comprises A, D, F, N, or S, X₁₀₀ comprises P, X₁₀₁ comprises I, L, N, P, V, or Y, and X₁₀₂ comprises G, L, N, S, T, or V.

In certain embodiments, the antigen binding protein comprises an HCDR1 which comprises FX₂₈FRDYAMT, wherein X₂₈ comprises N, P, or T; an HCDR2 which comprises X₅₀IX₅₂GSGGX₅₇TYYADAVKG, wherein X₅₂₀ comprises F, L, M, S, or T, X₅₂ comprises N, or S, and X₅₇ comprises K, or N; an HCDR3 which comprises X₉₈DRX₁₀₁X₁₀₂X₁₀₃TIRPX₁₀₈YX₁₁₀GX₁₁₂X₁₁₃X₁₁₄, wherein X₉₈ comprises K, or V, X₁₀₁ comprises K, L, or M, X₁₀₂ comprises S or T, X₁₀₃ comprises I, K, or R, X₁₀₈ comprises R, or Q, X₁₁₀ comprises L, R, or Y, X₁₁₂ comprises L, or M, X₁₁₃ comprises D, S, or W, and X₁₁₄ comprises G, H, K, Q, or V; and LCDR1 which comprises X₂₇SLX₃₀YSX₃₃GX₃₅NYLD, wherein X₂₇ comprises E, H, L, Q, or T, X₃₀ comprises A, L, or T, X₃₃ comprises A, I, P, or V, and X₃₅ comprises M, or Y; an LCDR2 which comprises X₅₅GX₅₇KRX₆₀S, wherein X₅₅ comprises L, of M, X₅₇ comprises H, N, S, or T, and X₆₀ comprises A, L, or M; and an LCDR3 which comprises MX₉₅X₉₆X₉₇X₉₈X₉₉PX₁₀₁X₁₀₂, wherein X₉₅ comprises N, or Q, X₉₆ comprises A, G, or P, X₉₇ comprises I or L, X₉₈ comprises Q, or R, X₉₉ comprises A, D, S, or T, X₁₀₀ comprises P, X₁₀₁ comprises L, N, or Y, and X₁₀₂ comprises G, L, N, S, or T.

In certain embodiments, the antigen binding protein comprises an HCDR1 which comprises SEQ ID NO:104, or SEQ ID NO:106, or SEQ ID NO:107; an HCDR2 which comprises SEQ ID NO:83, or SEQ ID NO:108, or SEQ ID NO:109, or SEQ ID NO:110; and HCDR3 which comprises SEQ ID NO:86, or SEQ ID NO:111, or SEQ ID NO:112, or SEQ ID NO:113, or SEQ ID NO:114, or SEQ ID NO:115, or SEQ ID NO:116, or SEQ ID NO:117, or SEQ ID NO:118, or SEQ ID NO:119, or SEQ ID NO:120, or SEQ ID NO:121; an LCDR1 which comprises SEQ ID NO:88, or SEQ ID NO:122, or SEQ ID NO:123, or SEQ ID NO:124, or SEQ ID NO:125, or SEQ ID NO:126, or SEQ ID NO:127, or SEQ ID NO:128, or SEQ ID NO:129, or SEQ ID NO:130, or SEQ ID NO:131, or SEQ ID NO:132; an LCDR2 which comprises SEQ ID NO:90, or SEQ ID NO:133, or SEQ ID NO:134, or SEQ ID NO:135, or SEQ ID NO:136, or SEQ ID NO:137; and/or and LCDR3 which comprises SEQ ID NO:101, or SEQ ID NO:138, or SEQ ID NO:139, or SEQ ID NO:140, or SEQ ID NO:141, or SEQ ID NO:142, or SEQ ID NO:143, or SEQ ID NO:144.

In certain embodiments, HCDR1 comprises FTFRDYAMT; and/or HCDR2 comprises LIX₅₂GSGGKTYYADAVKG, wherein X₅₂ comprises N, or S; and/or HCDR3 comprises DRX₁₀₁TKTIRPQYYGLWX₁₁₄, wherein X₁₀₁ comprises K or L, and X₁₁₄ comprises H or V; and/or LCDR1 comprises QSLX₃₀YSIGYNYLD, wherein X₃₀ comprises L, or T; and/or LCDR2 comprises LGX₅₇KRAS, wherein X₅₇ comprises H or S; and/or LCDR3 comprises MQX₉₆LGQPLX₁₀₂, wherein X₉₆ comprises G or P, and X₁₀₂ comprises G, L, N, S, or T.

In certain embodiments, the antigen binding protein comprises a heavy chain variable domain at least 80%, at least 85%, at least 90%, at least 95%, at least 97%, or 100% identical to a heavy chain variable domain of SEQ ID NO:5, SEQ ID NO:7, SEQ ID NO:15, SEQ ID NO:16, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:30, SEQ ID NO:33, SEQ ID NO:34, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:57, SEQ ID NO:58, SEQ ID NO:59, SEQ ID NO:63, SEQ ID NO:64, SEQ ID NO:65, SEQ ID NO:66, SEQ ID NO:67, SEQ ID NO:68, SEQ ID NO:69, SEQ ID NO:72, SEQ ID NO:73, SEQ ID NO:74, SEQ ID NO:75, SEQ ID NO:76, or SEQ ID NO:78, and/or a light chain variable domain at least 80%, at least 85%, at least 90%, at least 95%, at least 97%, or 100% identical to a light chain variable domain of SEQ ID NO:6, SEQ ID NO:8, SEQ ID NO:17, SEQ ID NO:18, SEQ ID NO:19, SEQ ID NO:20, SEQ ID NO:21, SEQ ID NO:22, SEQ ID NO:29, SEQ ID NO:31, SEQ ID NO:32, SEQ ID NO:35, SEQ ID NO:43, SEQ ID NO:56, SEQ ID NO:60, SEQ ID NO:61, SEQ ID NO:62, SEQ ID NO:68, SEQ ID NO:70, SEQ ID NO:71, SEQ ID NO:77, or SEQ ID NO:79.

In certain embodiments, HCDR1 comprises X₂₇X₂₈FRDX₃₂AMX₃₅, wherein X₂₇ comprises F or L, X₂₈ comprises P or T, X₃₂ comprises F or Y, and X₃₅ comprises A or T; HCDR2 comprises X₅₀X₅₁X₅₂GX₅₄X₅₅X₅₆X₅₇X₅₅YYADAVKG, wherein X₅₂₀ comprises L or T, X₅₁ comprises I or V, X₅₂ comprises N, S, or T, X₅₄ comprises D, N, S, or T, X₅₅ comprises A, G, or S, X₅₆ comprises G, S, or T, X₅₇ comprises K or N, and X₅₈ comprises T or V; HCDR3 comprises X₉₈DRLX₁₀₂X₁₀₃TX₁₀₅RPX₁₀₈YYGX₁₁₂X₁₃X₁₁₄, wherein X₉₈ comprises K or V, X₁₀₂ comprises S or T, X₁₀₃ comprises I or K, X₁₀₅ comprises I or V, X₁₀₈ comprises Q or R, X₁₁₂ comprises L or M, X₁₁₃ comprises D, S, or W, and X₁₁₄ comprises A, S, or V; LCDR1 comprises RX₂₅X₂₆X₂₇SLLYSIGYNYLD, wherein X₂₅ comprises A or S, X₂₆ comprises D or S, and X₂₇ comprises K or Q; LCDR2 comprises LGSKARAS; and LCDR3 comprises MQX₉₆X₉₇X₉₈X₉₉PX₁₀₁X₁₀₂, wherein X₉₆ comprises A or G, X₉₇ comprises I or L, X₉₈ comprises H, Q, or R, X₉₉ comprises A, D, or S, X₁₀₁ comprises L or Y, and X₁₀₂ comprises S or T.

In certain embodiments, HCDR1 comprises SEQ ID NO:92, HCDR2 comprises SEQ ID NO:108, and HCDR3 comprises SEQ ID NO:248. In certain embodiments, HCDR1 comprises SEQ ID NO:92, HCDR2 comprises SEQ ID NO:108, and HCDR3 comprises SEQ ID NO:250. In certain embodiments, HCDR1 comprises SEQ ID NO:92, HCDR2 comprises SEQ ID NO:212, and HCDR3 comprises SEQ ID NO:244. In certain embodiments, HCDR1 comprises SEQ ID NO:158, HCDR2 comprises SEQ ID NO:216, and HCDR3 comprises SEQ ID NO:97. In certain embodiments, HCDR1 comprises SEQ ID NO:92, HCDR2 comprises SEQ ID NO:108, and HCDR3 comprises SEQ ID NO:252. In certain embodiments, HCDR1 comprises SEQ ID NO:158, HCDR2 comprises SEQ ID NO:108, and HCDR3 comprises SEQ ID NO:97. In certain embodiments, HCDR1 comprises SEQ ID NO:92, HCDR2 comprises SEQ ID NO:202, and HCDR3 comprises SEQ ID NO:97. In certain embodiments, HCDR1 comprises SEQ ID NO:92, HCDR2 comprises SEQ ID NO:200, and HCDR3 comprises SEQ ID NO:97. In certain embodiments, HCDR1 comprises SEQ ID NO:92, HCDR2 comprises SEQ ID NO:108, and HCDR3 comprises SEQ ID NO:97. In certain embodiments, HCDR1 comprises SEQ ID NO:92, HCDR2 comprises SEQ ID NO:203, and HCDR3 comprises SEQ ID NO:97. In certain embodiments, HCDR1 comprises SEQ ID NO:92, HCDR2 comprises SEQ ID NO:195, and HCDR3 comprises SEQ ID NO:97. In certain embodiments, HCDR1 comprises SEQ ID NO:92, HCDR2 comprises SEQ ID NO:211, and HCDR3 comprises SEQ ID NO:97. In certain embodiments, HCDR1 comprises SEQ ID NO:185, HCDR2 comprises SEQ ID NO:83, and HCDR3 comprises SEQ ID NO:97. In certain embodiments, HCDR1 comprises SEQ ID NO:92, HCDR2 comprises SEQ ID NO:197, and HCDR3 comprises SEQ ID NO:97. In certain embodiments, HCDR1 comprises SEQ ID NO:92, HCDR2 comprises SEQ ID NO:83, and HCDR3 comprises SEQ ID NO:248. In certain embodiments, HCDR1 comprises SEQ ID NO:156, HCDR2 comprises SEQ ID NO:83, and HCDR3 comprises SEQ ID NO:97. In certain embodiments, HCDR1 comprises SEQ ID NO:186, HCDR2 comprises SEQ ID NO:83, and HCDR3 comprises SEQ ID NO:97. In certain embodiments, HCDR1 comprises SEQ ID NO:92, HCDR2 comprises SEQ ID NO:196, and HCDR3 comprises SEQ ID NO:97. In certain embodiments, HCDR1 comprises SEQ ID NO:92, HCDR2 comprises SEQ ID NO:95, and HCDR3 comprises SEQ ID NO:94. In certain embodiments, HCDR1 comprises SEQ ID NO:92, HCDR2 comprises SEQ ID NO:83, and HCDR3 comprises SEQ ID NO:98. In certain embodiments, LCDR1 comprises SEQ ID NO:88, LCDR2 comprises SEQ ID NO:90, and LCDR3 comprises SEQ ID NO:338. In certain embodiments, LCDR1 comprises SEQ ID NO:263, LCDR2 comprises SEQ ID NO:90, and LCDR3 comprises SEQ ID NO:339. In certain embodiments, LCDR1 comprises SEQ ID NO:88, LCDR2 comprises SEQ ID NO:90, and LCDR3 comprises SEQ ID NO:339. In certain embodiments, LCDR1 comprises SEQ ID NO:88, LCDR2 comprises SEQ ID NO:90, and LCDR3 comprises SEQ ID NO:345. In certain embodiments, LCDR1 comprises SEQ ID NO:88, LCDR2 comprises SEQ ID NO:90, and LCDR3 comprises SEQ ID NO:141. In certain embodiments, LCDR1 comprises SEQ ID NO:105, LCDR2 comprises SEQ ID NO:90, and LCDR3 comprises SEQ ID NO:102. In certain embodiments, LCDR1 comprises SEQ ID NO:105, LCDR2 comprises SEQ ID NO:90, and LCDR3 comprises SEQ ID NO:103.

In certain embodiments, the antigen binding protein comprises a heavy chain variable domain at least 80%, at least 85%, at least 90%, at least 95%, at least 97%, or 100% identical to SEQ ID NO:23, SEQ ID NO:24, SEQ ID NO:25, SEQ ID NO:26, SEQ ID NO:27, SEQ ID NO:28, SEQ ID NO:30, SEQ ID NO:33, SEQ ID NO:34, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:57, SEQ ID NO:58, SEQ ID NO:59, SEQ ID NO:63, SEQ ID NO:64, SEQ ID NO:65, SEQ ID NO:66, SEQ ID NO:67, SEQ ID NO:69, SEQ ID NO:72, SEQ ID NO:73, SEQ ID NO:74, SEQ ID NO:75, SEQ ID NO:76, or SEQ ID NO:78 and/or a light chain variable domain at least 80%, at least 85%, at least 90%, at least 95%, at least 97%, or 100% identical to SEQ ID NO:29, SEQ ID NO:31, SEQ ID NO:32, SEQ ID NO:35, SEQ ID NO:43, SEQ ID NO:56, SEQ ID NO:60, SEQ ID NO:61, SEQ ID NO:62, SEQ ID NO:68, SEQ ID NO:70, SEQ ID NO:71, SEQ ID NO:77, or SEQ ID NO:79.

In certain embodiments, HCDR1 comprises SEQ ID NO:358, HCDR2 comprises SEQ ID NO:83, and HCDR3 comprises SEQ ID NO:361. In certain embodiments, HCDR1 comprises SEQ ID NO:359, HCDR2 comprises SEQ ID NO:83, and HCDR3 comprises SEQ ID NO:87. In certain embodiments, HCDR1 comprises SEQ ID NO:360, HCDR2 comprises SEQ ID NO:83, and HCDR3 comprises SEQ ID NO:87. In certain embodiments, HCDR1 comprises SEQ ID NO:359, HCDR2 comprises SEQ ID NO:83, and HCDR3 comprises SEQ ID NO:361. In certain embodiments, HCDR1 comprises SEQ ID NO:107, HCDR2 comprises SEQ ID NO:83, and HCDR3 comprises SEQ ID NO:362. In certain embodiments, HCDR1 comprises SEQ ID NO:104, HCDR2 comprises SEQ ID NO:83, and HCDR3 comprises SEQ ID NO:362. In certain embodiments, HCDR1 comprises SEQ ID NO:358, HCDR2 comprises SEQ ID NO:83, and HCDR3 comprises SEQ ID NO:87. In certain embodiments, LCDR1 comprises SEQ ID NO:363, LCDR2 comprises SEQ ID NO:90, and LCDR3 comprises SEQ ID NO:338. In certain embodiments, LCDR1 comprises SEQ ID NO:263, LCDR2 comprises SEQ ID NO:90, and LCDR3 comprises SEQ ID NO:339. In certain embodiments, LCDR1 comprises SEQ ID NO:363, LCDR2 comprises SEQ ID NO:90, and LCDR3 comprises SEQ ID NO:365. In certain embodiments, LCDR1 comprises SEQ ID NO:364, LCDR2 comprises SEQ ID NO:90, and LCDR3 comprises SEQ ID NO:338. In certain embodiments, LCDR1 comprises SEQ ID NO:363, LCDR2 comprises SEQ ID NO:90, and LCDR3 comprises SEQ ID NO:139. In certain embodiments, LCDR1 comprises SEQ ID NO:88, LCDR2 comprises SEQ ID NO:90, and LCDR3 comprises SEQ ID NO:101.

In certain embodiments, the antigen binding protein comprises a heavy chain variable domain at least 80%, at least 85%, at least 90%, at least 95%, at least 97%, or 100% identical to SEQ ID NO:11, SEQ ID NO:12, SEQ ID NO:13, SEQ ID NO:14, SEQ ID NO:15, or SEQ ID NO:16 and/or a light chain variable domain at least 80%, at least 85%, at least 90%, at least 95%, at least 97%, or 100% identical to SEQ ID NO:17, SEQ ID NO:18, SEQ ID NO:19, SEQ ID NO:20, SEQ ID NO:21, or SEQ ID NO:22.

In certain embodiments, the heavy chain variable domain comprises SEQ ID NO:13 and the light chain variable domain comprises SEQ ID NO:19. In certain embodiments, the heavy chain variable domain comprises SEQ ID NO:13 and the light chain variable domain comprises SEQ ID NO:17. In certain embodiments, the heavy chain variable domain comprises SEQ ID NO:13 and the light chain variable domain comprises SEQ ID NO:21. In certain embodiments, the heavy chain variable domain comprises SEQ ID NO:15 and the light chain variable domain comprises SEQ ID NO:19. In certain embodiments, the heavy chain variable domain comprises SEQ ID NO:15 and the light chain variable domain comprises SEQ ID NO:17. In certain embodiments, the heavy chain variable domain comprises SEQ ID NO:15 and the light chain variable domain comprises SEQ ID NO:21. In certain embodiments, the heavy chain variable domain comprises SEQ ID NO:16 and the light chain variable domain comprises SEQ ID NO:12.

In certain embodiments, the antigen binding protein comprises no more than two (2) changes in any VH-CDR as compared to SEQ ID NO:15 and no more than two (2) changes in any VL-CDR as compared to SEQ ID NO:19.

In certain embodiments, the antigen binding protein comprises no more than one (1) changes in any VH-CDR as compared to SEQ ID NO:15 and no more than one (1) change in any VL-CDR as compared to SEQ ID NO:19.

In certain embodiments, the antigen binding protein comprises a heavy chain variable domain framework (HFR1+HFR2+HFR3+HFR4) at least 75%, or at least 80%, or at least 85%, or at least 90%, or at least 93%, or at least 95% identical, or identical to the heavy chain variable domain framework of SEQ ID NO:15.

In certain embodiments, the antigen binding protein comprises a light chain variable domain framework (LFR1+LFR2+LFR3+LFR4) at least 75%, or at least 80%, or at least 85%, or at least 90%, or at least 93%, or at least 95% identical, or identical to the light chain variable domain framework of SEQ ID NO:19.

In certain embodiments, the IL-4RA binding protein comprises one or more VH CDR substitutions from Table 4 or one or more VK CDR substitutions from Table 5, or one or more one or more VH CDR substitutions from Table 4 and one or more VK CDR substitutions from Table 5. In certain embodiments, the IL-4RA binding protein comprises one or more VH CDR substitutions from Table 10, or one or more VK CDR substitutions from Table 11, or one or more VH CDR substitutions from Table 10 and one or more VK CDR substitutions from Table 11. In certain embodiments, the IL-4RA binding protein comprises one or more VH CDR substitutions from Table 15, or one or more VL CDR substitutions from Table 15, or one or more VH CDR substitutions from Table 15 and one or more VL CDR substitutions from Table 15.

In certain embodiments, the IL-4RA binding protein comprises one or more VH CDRs from Table 4 or one or more VK CDRs from Table 5, or one or more one or more VH CDRs from Table 4 and one or more VK CDRs from Table 5. In certain embodiments, the IL-4RA binding protein comprises one or more VH CDRs from Table 10, or one or more VK CDRs from Table 11, or one or more VH CDRs from Table 10 and one or more VK CDRs from Table 11. In certain embodiments, the IL-4RA binding protein comprises one or more VH CDRs from Table 15, or one or more VL CDRs from Table 15, or one or more VH CDRs from Table 15 and one or more VL CDRs from Table 15.

In certain embodiments, the IL-4RA binding protein comprises the VH CDRs of a heavy chain variable domain of Table 15 and the VL CDRs of a light chain variable domain of Table 15. In certain embodiments, the IL-4RA binding protein comprises a heavy chain variable domain at least 80%, at least 85%, at least 90%, at least 95%, at least 97%, or 100% identical to a heavy chain variable domain of Table 15 and a light chain variable domain at least 80%, at least 85%, at least 90%, at least 95%, at least 97%, or 100% identical to a light chain variable domain of Table 15.

In certain embodiments, the IL-4RA binding protein comprises one or more HCDRs from Table 21 and one or more LCDRs from Table 22. In certain embodiments, the IL-4RA binding protein comprises the HCDRs of a heavy chain variable domain of Table 24 or the LCDRs of a light chain variable domain of Table 25, or the HCDRs of a heavy chain variable domain of Table 24 and the LCDRs of a light chain variable domain of Table 25.

In certain embodiments, an IL-4RA binding protein comprises the HCDRs of a heavy chain variable domain of Table 26, or the LCDRs of a light chain variable domain of Table 27, or the HCDRs of a heavy chain variable domain of Table 26 and the LCDRs of a light chain variable domain of Table 27. In certain embodiments, an IL-4RA binding protein comprises a heavy chain variable domain at least 80%, at least 85%, at least 90%, at least 95%, at least 97%, or 100% identical to SEQ ID NO:11, SEQ ID NO:12, SEQ ID NO:13, SEQ ID NO:14, SEQ ID NO:15, or SEQ ID NO:16, or a light chain variable domain at least 80%, at least 85%, at least 90%, at least 95%, at least 97%, or 100% identical to SEQ ID NO:17, SEQ ID NO:18, SEQ ID NO:19, SEQ ID NO:20, SEQ ID NO:21, or SEQ ID NO:22, or a heavy chain variable domain at least 80%, at least 85%, at least 90%, at least 95%, at least 97%, or 100% identical to SEQ ID NO:11, SEQ ID NO:12, SEQ ID NO:13, SEQ ID NO:14, SEQ ID NO:15, or SEQ ID NO:16 and a light chain variable domain at least 80%, at least 85%, at least 90%, at least 95%, at least 97%, or 100% identical to SEQ ID NO:17, SEQ ID NO:18, SEQ ID NO:19, SEQ ID NO:20, SEQ ID NO:21, or SEQ ID NO:22.

In certain embodiments, an anti-IL-4RA binding protein of the invention comprises the HCDRs of GFmix_PTH-H (SEQ ID NO:15) and the LCDRs of GFmix-THS-L (SEQ ID NO:19). In certain embodiments, an anti-IL-4RA binding protein of the invention comprises the HCDRs of GFmix_PTH-H (SEQ ID NO:15) and the LCDRs of GFmix3_KTHS-L (SEQ ID NO:21). In certain embodiments, an anti-IL-4RA binding protein of the invention comprises the HCDRs of 21188-H (SEQ ID NO:16) and the LCDRs of 21188-L (SEQ ID NO:22).

IL-4RA binding proteins disclosed herein by nature of their development comprise similar CDRs and variable domains, thus the VH CDRs are expected to be compatible with one another and the V_{L} CDRs are expected to be compatible with one another and the VH heavy chain variable domains are expected to be compatible with the V_{L} light chain variable domains. Accordingly, one or more CDRs of an IL-4RA binding protein herein may be substituted by a variety of corresponding CDRs herein, one of more amino acids in CDRs and FRs may be substituted by corresponding amino acids of CDRs and FRs herein, and V_{H} and V_{L} variable domains may be mixed and matched. In certain embodiments, an IL4A binding protein of the invention comprises a V_{H} sequence and a V_{L} sequence based on a particular IL-4RA binding protein exemplified or set forth herein and substituted with one or more other corresponding CDRs or amino acid substitutions set forth herein. Such CDRs and amino acids are found, for example, without limitation in Tables 4, 5, 10, 11, 12, 15, 21, 22, 24, 25, 26, and 27 and V_{H} and V_{L} sequences disclosed herein. In certain embodiments, the IL-4RA binding proteins include one or more conservative substitutions by comparison to CDRs and V_{H} and V_{L} sequences disclosed herein.

IL-4RA binding proteins of the invention do not include the V_{H} and Vκ domains of dupilumab.

In an aspect, the invention provides an isolated nucleic acid sequence encoding any one of the aforementioned anti-IL-4RA antibodies or antibody fragments, and a vector comprising or capable of expressing any one of the anti-IL-4RA antibodies or antibody fragments.

In another aspect, the invention provides a recombinant cell which comprises a nucleic acid sequence encoding any one of the aforementioned anti-IL-4RA antibodies or antibody fragments, or a vector comprising or capable of expressing any one of the anti-IL-4RA antibodies or antibody fragments.

The invention provides a method of producing any one of the aforementioned anti-IL-4RA antibodies or antibody fragments, which comprises culturing the cell capable of expressing the anti-IL-5 antibody or antibody fragment under conditions that result in production of the antibody or antibody fragment.

The invention provides a pharmaceutical composition comprising a therapeutically effective amount of any one of the aforementioned anti-IL-4RA antibodies or antibody fragments.

In an aspect, the invention provides a method of detecting IL-4RA in a sample comprising incubating the sample with any one of the aforementioned anti-IL-4RA antibodies or antibody fragments and detecting the anti IL-4RA antibody or antibody fragment bound to IL-4RA in the sample.

In an aspect, the invention provides a method of blocking binding of IL-4 to IL-4RA in a sample comprising incubating the sample with any one of the aforementioned anti-IL-4RA antibodies or antibody fragments and detecting whether IL-4 binds to IL-4RA.

In an aspect, the invention provides a method of blocking binding of IL-4 to a type 1 IL-4 receptor in a sample comprising incubating the sample with any one of the aforementioned anti-IL-4RA antibodies or antibody fragments and detecting whether IL-4 binds to the type 1 IL-4 receptor.

In an aspect, the invention provides a method of blocking binding of IL-4 to a type 2 IL-4 receptor (aka “shared IL-4 receptor”) in a sample comprising incubating the sample with any one of the aforementioned anti-IL-4RA antibodies or antibody fragments and detecting whether IL-13 binds to the type 2 IL-4 receptor.

In an aspect, the invention provides a method of blocking binding of IL-13 to a type 2 IL-4 receptor (aka “shared IL-4 receptor”) in a sample comprising incubating the sample with any one of the aforementioned anti-IL-4RA antibodies or antibody fragments and detecting whether IL-13 binds to the type 2 IL-4 receptor.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts the functional assay testing antibodies ability to block canine IL-4 signaling through canine IL-4 receptor alpha (IL-4RA). Canine IL-4RA was measured using the phospho-STAT6 assay, which measures the tyrosine phosphorylation of STAT6.

FIG. 2 depicts the functional assay testing antibodies ability to block canine IL-13 signaling through canine IL-4 receptor alpha (IL-4RA). Canine IL-4RA was measured using the phospho-STAT6 assay, which measures the tyrosine phosphorylation of STAT6.

FIG. 3 (SEQ ID NO: 5, 7, 76, 78, 9, 11-16) depicts heavy chain variable domains (VH) of selected binding proteins of the invention. For reference, amino acid positions are numbered by IMGT position and starting from the amino terminal. CDRs as depicted encompass Kabat, Chothia, and IMGT CDRs.

FIG. 4 (SEQ ID NO: 6, 8, 77, 79, 10, 17-22) depicts light chain variable domains (VL) of selected binding proteins of the invention. For reference, amino acid positions are numbered by IMGT position and starting from the amino terminal. CDRs as depicted encompass Kabat, Chothia, and IMGT CDRs.

### DETAILED DESCRIPTION OF THE INVENTION

The invention provides novel anti-IL-4RA proteins and antibodies that are suitable for administration to a human or canine subject. To facilitate an understanding of the present technology, a number of terms and phrases are defined below. Additional terminology is set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present invention also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present invention shall have the meanings that are commonly understood by those of ordinary skill in the art. For example, any nomenclature used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The phrase “and/or” is used herein. To avoid confusion, the phrase is used to indicate at either one or the other of both of two conditions. Used with three or more conditions, the phrase means that one or more of the conditions are met. When referring to details of the invention, the term and/or contemplates, supports and may be substituted with either of the terms “and” or “or.”

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%. 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

As used herein, a “nucleic acid” or a “nucleic acid sequence” refers to a polymer or oligomer of pyrimidine and/or purine bases, preferably cytosine, thymine, and uracil, and adenine and guanine, respectively. The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506, incorporated herein by reference), locked nucleic acid (LNA; see Wahlestedt et al., Proc. Natl. Acad. Sci. U.S.A., 97: 5633-5638 (2000), incorporated herein by reference), cyclohexenyl nucleic acids (see Wang, J. Am. Chem. Soc., 122: 8595-8602 (2000), incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics when compared to the wild-type gene or gene product.

The invention provides an antigen binding protein that specifically binds to interleukin-4 receptor alpha (IL-4RA). In certain embodiments, the IL-4RA binding protein comprises: (a) a heavy chain complementarity determining region 1 (HCDR1) comprising X₂₇X₂₈X₂₉X₃₀X₃₁X₃₂X₃₃X₃₄X₃₅, wherein X₂₇ comprises F, I, L, or V, X₂₈ comprises A, D, I, N, P, R, S, or T, X₂₉ comprises F or S, X₃₀ comprises D, G, N, R, or S, X₃₁ comprises D, X₃₂ comprises F or Y, X₃₃ comprises A, X₃₄ comprises M, and X₃₅ comprises A, S, T, or V; (b) a heavy chain complementarity determining region 2 (HCDR2) comprising X₅₀X₅₁X₅₂X₅₃X₅₄X₅₅X₅₆X₅₇X₅₈X₅₉X₆₀X₆₁X₆₂X₆₃X₆₄X₆₅X₆₆, wherein X₅₀ comprises F, L, M, S, or T, X₅₁ comprises I or V, X₅₂ comprises N, S, or T, X₅₃ comprises G, X₅₄ comprises D, I, N, S, or T, X₅₅ comprises A, G, or S, X₅₆ comprises D, G, N, S, or T, X₅₇ comprises G, K, N, R, or S, X₅₈ comprises I, S, T, or V, X₅₉ comprises Y, X₆₀ comprises Y, X₆₁ comprises A, X₆₂ comprises D, X₆₃ comprises A, X₆₄ comprises V X₆₅ comprises K, and X₆₆ comprises D or G; (c) a heavy chain complementarity determining region 3 (HCDR3) comprising X₉₈X₉₉X₁₀₀X₁₀₁X₁₀₂X₁₀₃X₁₀₄X₁₀₅X₁₀₆X₁₀₇X₁₀₈X₁₀₉X₁₁₁X₁₁₂X₁₁₃X₁₁₄, wherein X₉₈ comprises A, I, K, R, S, T, or V, X₉₉ comprises D, X₁₀₀ comprises R, X₁₀₁ comprises I, K, L, or M, X₁02 comprises S or T, X₁₀₃ comprises I, H, N, K, or R, X₁₀₄ comprises M or T, X₁₀₅ comprises I or V, X₁₀₆ comprises R, X₁₀₇ comprises P, X₁₀₈ comprises A, Q, R, or V, X₁₀₉ comprises Y, X₁₁₀ comprises A, E, F, L, M, N, R, S, T, Q, or Y, X₁₁₁ comprises G, X₁₁₂ comprises F, L, or M, X₁₁₃ comprises D, Q, S, or W, and X₁₁₄ comprises, A, D, F, G, H, L, Q, R, S, or V; (d) a light chain complementarity determining region 1 (LCDR1) comprising X₂X₂₈X₂₉X₃₀X₃₁X₃₂X₃₃X₃₄X₃₅X₃₆X₃₇X₃₈X₃₉, wherein X₂₇ comprises E, G, H, K, L, Q, R, or T, X₂₈ comprises S or T, X₂₉ comprises L or V, X₃₀ comprises A, I, L, Q, S, or T, X₃₁ comprises Y, X₃₂ comprises G or S, X₃₃ comprises A, G, I, N, P, S, T, or V, X₃₄ comprises D, G, L, or R, X₃₅ comprises A, F, I, K, L, M, N, R, S, T, or Y, X₃₆ comprises N, X₃₇ comprises Y, X₃₈ comprises I, L, or V, and X₃₉ comprises D; (e) a light chain complementarity determining region 2 (LCDR2) comprising X₅₅X₅₆X₅₇X₅₈X₅₉X₆₀X₆₁, wherein X₅₅ comprises A, H, L, M, N, or Q, X₅₆ comprises G or S, X₅₇ comprises D, H, N, S, T, or Y, X₅₈ comprises K, N, or R, X₅₉ comprises L or R, X₆₀ comprises A, M, Q, S, or Y, and X₆₁ comprises A, G, P, Q, R, S, or T; and (f) a light chain complementarity determining region 3 (LCDR3) comprising X₉₄X₉₅X₉₆X₉₇X₉₈X₉₉X₁₀₀X₁₁X₁₀₉, wherein X₉₄ comprises M, X₉₅ comprises N, or Q, X₉₆ comprises A, G, N, P, or S, X₉₇ comprises I or L, X₉₈ comprises F, H, I, Q, R, S, or T, X₉₉ comprises A, D, F, N, or S, X₁₀₀ comprises P, X₁₀₁ comprises I, L, N, P, V, or Y, and X₁₀₂ comprises G, L, N, S, T, or V.

In certain embodiments, the antigen binding protein of claim 1 comprises an HCDR1 which comprises FX₂₈FRDYAMT, wherein X₂₈ comprises N, P, or T; an HCDR2 which comprises X₅₀IX₅₂GSGGX₅₇TYYADAVKG, wherein X₅₀ comprises F, L, M, S, or T, X₅₂ comprises N, or S, and X₅₇ comprises K, or N; an HCDR3 which comprises X₉₈DRX₁₀₁X₁₀₂X₁₀₃TIRPX₁₀₈YX₁₁₀GX₁₁₂X₁₁₃X₁₁₄, wherein X₉₈ comprises K, or V, X₁₀₁ comprises K, L, or M, X₁₀₂ comprises S or T, X₁₀₃ comprises I, K, or R, X₁₀₈ comprises R, or Q, X₁₁₀ comprises L, R, or Y, X₁₁₂ comprises L, or M, X₁₁₃ comprises D, S, or W, and X₁₁₄ comprises G, H, K, Q, or V; and LCDR1 which comprises X₂₇SLX₃₀YSX₃₃GX₃₅NYLD, wherein X₂₇ comprises E, H, L, Q, or T, X₃₀ comprises A, L, or T, X₃₃ comprises A, I, P, or V, and X₃₅ comprises M, or Y; an LCDR2 which comprises X₅₅GX₅₇KRX₆₀S, wherein X₅₅ comprises L, of M, X₅₇ comprises H, N, S, or T, and X₆₀ comprises A, L, or M; and an LCDR3 which comprises MX₉₅X₉₆X₉₇X₉₈X₉₉PX₁₀₁X₁₀₂, wherein X₉₅ comprises N, or Q, X₉₆ comprises A, G, or P, X₉₇ comprises I or L, X₉₈ comprises Q, or R, X₉₉ comprises A, D, S, or T, X₁₀₀ comprises P, X₁₀₁ comprises L, N, or Y, and X₁₀₂ comprises G, L, N, S, or T.

In certain embodiments, the IL-4RA binding protein comprises one or more VH CDRs or CDR substitutions from Table 4 (e.g., SEQ ID NO:92, SEQ ID NO:93, SEQ ID NO:83, SEQ ID NO:95, SEQ ID NO:96, SEQ ID NO:94, SEQ ID NO:97, SEQ ID NO:98), or one or more VK CDRs or CDR substitutions from Table 5 (e.g., SEQ ID NO:88, SEQ ID NO:90, SEQ ID NO:99, SEQ ID NO:100, SEQ ID NO:101, SEQ ID NO:102, SEQ ID NO:103) or one or more one or more VH CDRs or CDR substitutions from Table 4 and one or more VK CDRs or CDR substitutions from Table 5. Whereas Table 4 and Table 5 depict experimentally defined CDRs, Kabat, Chothia, and IMGT CDRs are also intended. Thus, in Table 4, the HCDR1 sequences includes Kabat HCDR1 at positions 31 to 35. Positions 26-32 correspond to Chothia HCDR1 and positions 26-33 correspond to IMGT HCDR1. The amino acid at position 26 is usually “G” as shown for example in Table 1. In Table 4, the HCDR2 sequences also depict Kabat HCDR2 at positions 50-66, Chothia HCDR2 at positions 52-57, and IMGT HCDR2 at positions 51-58. In Table 4, the HCDR3 sequences also depict Kabat HCDR3 at positions 99-114, Chothia HCDR3 at positions 99-114, and IMGT HCDR3 at positions 97-114. The amino acid at position 97 is usually “A” as shown in Table 1. In Table 5, the LCDR1 sequences includes Kabat LCDR1 at positions 24-39. Positions 24-39 correspond to Chothia LCDR1 and positions 27-37 correspond to IMGT LCDR1L. In Table 5, the HCDR2 sequences also depict Kabat LCDR2 at positions 55-61, Chothia LCDR2 at positions 55-61, and IMGT LCDR2 at positions 55-57. In Table 5, the LCDR3 sequences also depict Kabat LCDR3 at positions 94-102, Chothia HCDR3 at positions 94-102, and IMGT HCDR3 at positions 94-102.

In certain embodiments, the IL-4RA binding protein comprises one or more VH CDRs or VH-CDR substitutions from Table 10 (e.g., SEQ ID NO:104, SEQ ID NO:106, SEQ ID NO:107, SEQ ID NO:83, SEQ ID NO:108, SEQ ID NO:109, SEQ ID NO:110, SEQ ID NO:86, SEQ ID NO:11, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121), or one or more VK CDRs or one or more VK-CDR substitutions from Table 11 (e.g., SEQ ID NO:88, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:90, SEQ ID NO:133, SEQ ID NO:134, SEQ ID NO:135, SEQ ID NO:136, SEQ ID NO:137, SEQ ID NO:101, SEQ ID NO:138, SEQ ID NO:139, SEQ ID NO:140, SEQ ID NO:141, SEQ ID NO:142, SEQ ID NO:143, SEQ ID NO:144), or one or more VH CDRs or VH-CDR substitutions from Table 10 and one or more VK CDRs or one or more VK-CDR substitutions from Table 11.

In certain embodiments, the IL-4RA binding protein comprises one or more VH CDR substitutions from Table 15, or one or more VL CDR substitutions from Table 15, or one or more VH CDR substitutions from Table 15 and one or more VL CDR substitutions from Table 15. Table 15 depicts amino acid substitutions in the CDRs of TB_640_005. Those CDRs and the substituted CDRs are set forth in Table 10 and Table 11 and discussed above. The substitutions include, alone and in combination, VH substitutions S52N, L101K, and V114H, and VL substitutions L30T, S57H, G96P, T102S, T102G, and T102L.

According to certain exemplary embodiments of the present invention, the IL-4RA binding protein is an anti-IL-4RA antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the invention, the FRs of the antibody (or antigen-binding portion thereof) may be identical to the canine germline sequences, or may be naturally or artificially modified. An amino acid consensus sequence may be defined based on a side-by-side analysis of two or more CDRs.

Antibody residues that have a substantial impact on affinity and specificity of binding to target antigen are primarily located in CDRs. Kabat et al. compiled and aligned immunoglobulin heavy and light chain sequences and were the first to propose a standardized numbering scheme for the variable regions of immunoglobulins identifying conserved and hypervariable regions and residues. (Kabat E A et al., 1979, Sequences of Immunoglobulin Chains: Tabulation and Analysis of Amino Acid Sequences of Precursors, V-regions, C-regions, J-Chain and BP-Microglobulins, Department of Health, Education, and Welfare, Public Health Service, National Institutes of Health). While the Kabat system is a widely adopted standard for numbering antibody residues, the hypervariable regions defined by Kabat do not exactly match with the structural aspects of antigen-binding loops. Chothia and Lesk developed a structure-based numbering scheme by aligning crystal structures of antibody variable regions and classified CDR loops in a small number of “canonical” classes (Chothia C, et al., 1987, Canonical structures for the hypervariable regions of immunoglobulins. J. Mol. Biol. 196:901-17. doi: 10.1016/0022-2836(87)90412-8). An advantage of the Chothia numbering scheme is that topologically aligned residues from different antibodies are localized at the same position number and the Chothia CDR definition corresponds in most antibody sequences to the structural antigen-binding loop. Lefranc introduced a new system based on germ-line sequences intended to standardize numbering for all proteins of the immunoglobulin superfamily, including T cell receptor chains. (Giudicelli V et al., 1997, IMGT, the international ImMunoGeneTics database. Nucleic Acids Res. 25:206-11), which was then extended to entire variable domains (Lefranc M-P et al., 2003, IMGT unique numbering for immunoglobulin and T cell receptor variable domains and Ig superfamily V-like domains. Dev Comp Immunol. 27:55-77. doi: 10.1016/S0145-305X(02)00039-3). Additional numbering systems have been proposed to align unconventional frameworks (Abhinandan K R et al., 2008, Analysis and improvements to Kabat and structurally correct numbering of antibody variable domains. Mol Immunol. 45:3832-9. doi: 10.1016/j.molimm.2008.05.022) and to subdivide variable chain sequences into multiple fragments including structurally invariant “cores” (Gelfand et al., 1998, Algorithmic determination of core positions in the VL and VH domains of immunoglobulin molecules. J Comput Biol. (1998) 5:467-77). In certain embodiments of the invention, CDR residues are identified according to such a standard system as set forth above. In certain embodiments, antibodies of the invention are identified by all or a subset of Kabat CDR residues of the antibody sequences set forth herein. In certain embodiments, antibodies of the invention are identified by all or a subset of Chothia CDR residues of the antibody sequences set forth herein. In certain embodiments, antibodies of the invention are identified by all or a subset of IMGT CDR residues of the antibody sequences set forth herein. In certain embodiments, antibodies of the invention are identified by CDR residues defined by two or more systems, comprising e.g., but not limited to, all or a subset of residues of HCDR1 according to Kabat, all or a subset of residues of HCDR2 according to Chothia, all or a subset of residues of HCDR3 according to Kabat, all or a subset of residues of LCDR1 according to Kabat, all or a subset of residues of LCDR2 according to IMGT, and all or a subset of residues of LCDR3 according to Chothia. In certain embodiments, the extent of one or more CDRs of antibodies of the invention are set specifically set forth herein. For example, CDRs may be identified based on experimental observations of variable or fixed amino acids. In embodiments of the invention, framework amino acids are the amino acids that flank the CDRs. Frameworks may be referred together, to the exclusion of CDR amino acids, for example, “HFR1+HFR2+HFR3+HFR4” or “LFR1+LFR2+LFR3+LFR4.”

For reference, the following table shows relative locations of Kabat, Chothia, and IMGT CDRs mapped on caninized antibody TB640_005 VH (SEQ ID NO:7) and VL (SEQ ID NO:8) described herein that binds to canine IL-4RA. “X” represents CDR amino acid positions starting from the amino terminal of the antibody V_{H} or V_{L} chain as follows: X₂₆X₂₇X₂₈X₂₉X₃₀X₃₁X₃₂X₃₃X₃₄X₃₅ for HCDR1, X₅₀X₅₁X₅₂X₅₃X₅₄X₅₅X₅₆X₅₇X₅₈X₅₉X₆₀X₆₁X₆₂X₆₃X₆₄X₆₅X₆₆ for HCDR2, X₉₇X₉₈X₉₉X₁₀₀X₁₀₁X₁₀₂X₁₀₃X₁₀₄X₁₀₅X₁₀₆X₁₀₇X₁₀₈X₁₀₉X₁₁₀X₁₁₁X₁₂X₁₁₃X₁₁₄ for HCDR3, X₂₄X₂₅X₂₆X₂₇X₂₈X₂₉X₃₀X₃₁X₃₂X₃₃X₃₄X₃₅X₃₆X₃₇X₃₈X₃₉ for LCR1, X₅₅X₅₆X₅₇X₅₈X₅₉X₆₀X₆₁ for LCDR2, and X₉₄X₉₅X₉₆X₉₇X₉₈X₉₉X₁₀₀X₁₀₁X₁₀₂ for LCDR3. Antibodies comprising the CDRs can have longer or shorter frameworks. In such embodiments, it will be clear from published sources or by comparison to (e.g. alignment with) instantly disclosed antibodies which are CDR amino acids and which are frameworks.

| | HCDR1 | HCDR2 | HCDR3 |
|---|---|---|---|
| Kabat | DYAMT | LISGSGGKTYYADAVKG | DRLTKTIRPQYYGLWV |
| | (SEQ ID NO: 80) | (SEQ ID NO: 83) | (SEQ ID NO : 86) |
| Chothia | GFTFRDY | SGSGGK | DRLTKTIRPQYYGLWV |
| | (SEQ ID NO : 81) | (SEQ ID NO: 84) | (SEQ ID NO : 86) |
| IMGT | GFTFRDYA | ISGSGGKT | AVDRLTKTIRPQYYGLWV |
| | (SEQ ID NO: 82) | (SEQ ID NO: 85) | (SEQ ID NO: 87) |
| | XXXXXXXXXX | XXXXXXXXXXXXXXXXX | XXXXXXXXXXXXXXXXXX |
| | LCDR1 | LCDR2 | LCDR3 |
| Kabat | RSSQSLLYSIGYNYLD | LGSKRAS | MQGLQTPLT |
| | (SEQ ID NO:88) | (SEQ ID NO: 90) | (SEQ ID NO: 91) |
| Chothia | RSSQSLLYSIGYNYLD | LGSKRAS | MQGLQTPLT |
| | (SEQ ID NO: 88) | (SEQ ID NO: 90) | (SEQ ID NO: 91) |
| IMGT | QSLLYSIGYNY | LGS | MQGLQTPLT |
| | (SEQ ID NO: 89) | XXXXXXX | (SEQ ID NO: 91) |
| | XXXXXXXXXXXXXXXX | | XXXXXXXXX |

In one aspect, the invention provides a binding protein suitable for use in a mammal, for example, without limitation, a canine. In certain embodiments, a caninized anti-IL-4RA binding protein comprises a heavy chain complementarity determining region 1 (HCDR1), a heavy chain complementarity determining region 2 (HCDR2), a heavy chain complementarity determining region 3 (HCDR3), a light chain complementarity determining region 1 (LCDR1), a light chain complementarity region 2 (LCDR2), and a light chain complementarity region 3 (LCDR3). The CDRs may be defined according to Kabat, Chothia, IMGT, or as set forth herein.

In certain embodiments, the anti-IL-4RA binding protein comprises one or more (i.e. one, two, three, four, five, or all six) CDRs of a caninized anti-IL-4RA binding protein set forth herein. In certain embodiments, the anti-IL-4RA binding protein comprises one or more (i.e. one, two, three, four, five, or all six) CDRs of an affinity matured caninized antibody disclosed herein. Such CDRs may further comprise amino acid changes at one or two positions, for example incorporating an amino acid demonstrated herein to be compatible with IL-4RA binding or a conservative substitution.

In certain embodiments, a binding protein of the invention comprises a caninized antibody or is suitable for administration to a canine. In certain embodiments, a binding proteins of the invention comprises a felinized antibody or is suitable for administration to a feline. In certain embodiments, a binding protein of the invention comprises an equinized antibody or is suitable for administration to an equine. In certain embodiments, a binding proteins of the invention comprises a humanized antibody or is suitable for administration to a human.

In certain embodiments, an amino acid residue is mutated into one that allows the properties of the amino acid side-chain to be conserved. Examples of the properties of amino acid side chains comprise: polar amino acids (C, S, T, Y, N, Q), nonpolar amino acids (A, G, I, L, M, W, F, P, V), basic amino acids (H, K, R), acidic amino acids (E, D), hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)). The number of mutated amino acids is not limited, but in general, the number falls within 40% of amino acids of each CDR, and preferably within 35%, and still more preferably within 30% (e.g., within 25%). The identity of amino acid sequences can be determined as described herein.

The invention provides recombinant antibodies designed or modified to minimize antigenicity in canines and humans. In certain embodiments, the antibodies are further modified to remove T cell epitopes.

As used herein, the term “canine” refers to any member of the Canidae family. Domestic dogs, pure-bred and/or mongrel companion dogs, and wild or feral dogs are all canines.

As used herein the term “human framework” or “canine framework” refers to the amino acid sequence of the heavy chain and light chain of a canine antibody other than the hypervariable region residues defined herein as CDR residues. With regard to a humanized antibody, in certain embodiments, canine CDRs are identified in human antibody heavy and light chains variable domain sequences that closely match CDRs of IL-4RA-binding antibodies originating in other species. In certain embodiments, native human CDRs are replaced with the corresponding foreign CDRs (e.g., those from a rat or a mouse antibody) in both chains. With regard to a caninized antibody, in certain embodiments, canine CDRs are identified in canine antibody heavy and light chains variable domain sequences that closely match CDRs of IL-4RA-binding antibodies originating in other species. In certain embodiments, native canine CDRs are replaced with the corresponding foreign CDRs (e.g., those from a rat or a mouse antibody) in both chains. Optionally the heavy and/or light chains of the humanized or caninized antibody may contain some mutated or foreign non-CDR residues, e.g., framework amino acid residues that vary among germline antibody sequence or mutations that preserve the conformation of the foreign CDRs within the antibody.

Five major isotypes (IgA, IgG, IgM, IgD, IgE) and two forms of light chain (κ and λ) are present in dogs. In the dog, there are four subtypes for IgG, which are IgGA, IgGB, IgGC, and IgGD (Bergeron et al, 2014, Comparative functional characterization of canine IgG subclasses. Veterinary Immunology and Immunopathology. 157:31-41). For the cat, there are three subtypes of IgG which are IgG1a, IgG1b, and IgG2 (Streitzel et al. 2014, In vitro functional characterization of canine IgGs. Vet Immunol Immunopathol 158, 214-223, doi.org/10.1016/j.vetimm.2014.01.012).

In certain embodiments, antibodies of the invention, including but not limited to caninized, felinized, and humanized antibodies can be engineered to modulate one or more effector functions or circulation half-life. Hinge and constant domains of an antibody engage host receptors or complement protein to mediate effector functions and regulate antibody circulation. In certain embodiments, one or more effector functions is enhanced. In certain embodiments, one or more effector functions is reduced or eliminated. In certain embodiments, antibodies of the invention comprise modifications to modulate antibody-dependent cytotoxicity (ADCC) and/or complement-dependent cytotoxicity (CDC). A non-limiting example involves engineering of canine of eline IgG1a or IgG1b constant region residues Met242 and/or Leu243 (EU numbering) to reduce effector function (see, e.g., Lund et al., Human Fc gamma RI and Fc gamma RII interact with distinct but overlapping sites on human IgG. J Immunol., 1991, 147:2657-62). In certain embodiments, a canine or feline constant region of the invention comprises M242A and L243A substitution. In certain embodiments, the second constant domain (CH2) and/or the third constant domain (CH3) comprises mutations and combinations of mutations from wild-type designed to modulate binding to FcRn (neonatal Fc) receptor.

The term “antibody,” as used herein, includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. As used herein, the term “specifically binds” or “binds specifically” means that an IL-4RA binding protein of the invention reacts or associates more frequently, more rapidly, with greater duration and/or with greater affinity with IL-4RA than it does with alternative antigens. For example, IL-4RA binding protein binds to IL-4RA with materially greater affinity (e.g., at least 2-fold or 5-fold or 10-fold or 20-fold or 50-fold or 100-fold or 500-fold or 1000-fold or 10,000-fold or greater) than it does to other proteins or peptides. In certain embodiments, the IL-4RA-binding proteins binds to IL-4RA with an equilibrium dissociation constant KD for the epitope or target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that an antibody that specifically binds to a target (e.g., IL-4RA) from one species may also specifically bind to orthologs of IL-4RA.

Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

In certain embodiments, an antigen-binding fragment of an antibody comprises at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2, (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

The term “diabody (Db)” refers to a bivalent antibody fragment constructed by gene fusion (for example, P. Holliger et al., Proc. Natl. Acad. Sci. USA 90: 6444-6448 (1993), EP 404,097, WO 93/11161). In general, a diabody is a dimer of two polypeptide chains. In the each of the polypeptide chains, a light chain variable region (VL) and a heavy chain variable region (VH) in an identical chain are connected via a short linker, for example, a linker of about five residues, so that they cannot bind together. Because the linker between the two is too short, the VL and VH in the same polypeptide chain cannot form a single chain V region fragment, but instead form a dimer. Thus, a diabody has two antigen-binding domains. When the VL and VH regions against the two types of antigens (a and b) are combined to form VLa-VHb and VLb-VHa via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, preferably, a polypeptide linker (Huston, J. S. et al., Proc. Natl. Acad. Sci. U.S.A., 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The term “Kd” as used herein, refers to the dissociation constant of an antibody-antigen interaction. The dissociation constant, Kd, and the association constant, Ka, are quantitative measures of affinity. At equilibrium, free antigen (Ag) and free antibody (Ab) are in equilibrium with antigen-antibody complex (Ag-Ab), and the rate constants, ka and kd, quantitate the rates of the individual reactions. At equilibrium, ka [Ab][Ag]=kd [Ag-Ab]. The dissociation constant, Kd, is given by: Kd=kd/ka=[Ag][Ab]/[Ag-Ab]. Kd has units of concentration, most typically M, mM, nM, pM, etc. When comparing antibody affinities expressed as Kd, having greater affinity for IL-4RA is indicated by a lower value. The association constant, Ka, is given by: Ka=ka/kd=[Ag−Ab]/[Ag][Ab]. Ka has units of inverse concentration, most typically M-1, mM-1, nM-1, pM-1, etc. As used herein, the term “avidity” refers to the strength of the antigen-antibody binding taking valency into account.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

The anti-IL-4RA proteins described herein, including antibodies or fragments thereof, are useful for ameliorating, or reducing the symptoms of, or treating, or preventing, diseases or conditions associated, characterized by, or caused by altered levels of IL-4 and/or IL-13. Such diseases or conditions include, without limitation, atopic dermatitis, allergic dermatitis, pruritus, asthma, psoriasis, scleroderma, or eczema. The anti-IL-4RA proteins or fragments, as well as combinations with other agent, are to be administered in a therapeutically effective amount to subjects in need of such treatment in the form of a pharmaceutical composition as described herein.

In certain embodiments the method comprises ameliorating, or reducing the symptoms of, or treating, or preventing disease in a subject. In certain embodiments, treatment comprises administering the anti-IL-4RA proteins, antibodies, or fragments thereof alone. In certain embodiments, treatment comprises administering the anti-IL-4RA proteins, antibodies, or fragments thereof in conjunction with a second agent used to treat, ameliorate, reduce symptoms of, or prevent the same disease or disorder or to treat a lung disease, cardiovascular disease, cancer, infectious disease, neurological disease, allergic/inflammatory disease, or metabolic disease.

Nonlimiting examples of cardiovascular diseases the antibody compositions and methods are used for ameliorating, or reducing the symptoms of, or treating, or preventing include hypertension, cardiac toxicity of anti-cancer drugs, cardiac toxicity of anthracyclines, cardiac toxicity of quinolones, heart failure regardless of origin, ischemia, heart attack, stroke, atherosclerosis, cardiac fibrillation, thrombosis and embolism.

Nonlimiting examples of infectious diseases the antibody compositions and methods are used for ameliorating, or reducing the symptoms of, or treating, or preventing include AIDS, alveolar hydatid disease (AHD, echinococcosis), amebiasis (Entamoeba histolytica infection), Angiostrongylus infection, anisakiasis, anthrax, babesiosis (Babesia infection), Balantidium infection (balantidiasis), Baylisascaris infection (raccoon roundworm), bilharzia (schistosomiasis), Blastocystis hominis infection (blastomycosis), boreliosis, botulism, Brainerd diarrhea, brucellosis, bovine spongiform encephalopathy (BSE), candidiasis, capillariasis (Capillaria infection), chronic fatigue syndrome (CFS), Chagas disease (American trypanosomiasis), chickenpox (Varicella-Zoster virus), Chlamydia pneumoniae infection, cholera, Creutzfeldt-Jakob disease (CJD), clonorchiasis (Clonorchis infection), cutaneous larva migrans (CLM) (hookworm infection), coccidioidomycosis, conjunctivitis, Coxsackievirus A16 (hand, foot and mouth disease), cryptococcosis, Cryptosporidium infection (cryptosporidiosis), Culex mosquito (West Nile virus vector), cyclosporiasis (Cyclospora infection), cysticercosis (neurocysticercosis), Cytomegalovirus infection, Dengue/Dengue fever, Dipylidium infection (dog and cat flea tapeworm), Ebola virus hemorrhagic fever, encephalitis, Entamoeba coli infection, Entamoeba dispar infection, Entamoeba hartmanni infection, Entamoeba histolytica infection (amebiasis), Entamoeba polecki infection, enterobiasis (pinworm infection), enterovirus infection (non-polio), Epstein-Barr virus infection, Escherichia coli infection, foodborne infection, foot and mouth disease, fungal dermatitis, gastroenteritis, group A streptococcal disease, group B streptococcal disease, Hansen's disease (leprosy), Hantavirus pulmonary syndrome, head lice infestation (pediculosis), Helicobacter pylori infection, hematologic disease, Hendra virus infection, hepatitis (HCV, HBV), herpes zoster (shingles), HIV Infection, human ehrlichiosis, human parainfluenza virus infection, influenza, isosporiasis (Isospora infection), Lassa fever, leishmaniasis, Kala-azar (Kala-azar, Leishmania Infection), lice (body lice, head lice, pubic lice), Lyme disease, malaria, Marburg hemorrhagic fever, measles, meningitis, mosquito-borne diseases, Mycobacterium avium complex (MAC) infection, Naegleria infection, nosocomial infections, nonpathogenic intestinal ameobae infection, onchocerciasis (river blindness), opisthorciasis (Opisthorcis infection), parvovirus infection, plague, Pneumocystis carinii pneumonia (PCP), polio, Q fever, rabies, respiratory syncytial virus (RSV) Infection, rheumatic fever, Rift Valley fever, river blindness (onchocerciasis), rotavirus infection, roundworm infection, salmonellosis, Salmonella enteritidis, scabies, shigellosis, shingles, sleeping sickness, smallpox, streptococcal Infection, tapeworm infection (Taenia infection), tetanus, toxic shock syndrome, tuberculosis, ulcers (peptic ulcer disease), valley fever, Vibrio parahaemolyticus infection, Vibrio vulnificus infection, viral hemorrhagic fever, warts, waterborne infectious diseases, West Nile virus infection (West Nile encephalitis), whooping cough, yellow fever.

Nonlimiting examples of allergic/inflammatory conditions the antibody compositions and methods are used for ameliorating, or reducing the symptoms of, or treating, or preventing include, asthma, bronchial asthma, rheumatoid arthritis, inflammatory Bowel disease, type II diabetes, diabetes mellitus and deafness (DAD), Ballinger-Wallace syndrome, inflammatory diseases, rheumatic fever, pulmonary arterial hypertension, innate immune responses, cardiopulmonary diseases such as: chronic obstructive pulmonary disease, pulmonary embolism, pericarditis, coarctation of aorta, tetralogy of Fallot, aortic stenosis, mitral stenosis, aortic regurgitation, mitral regurgitation, pneumoconiosis, bronchiectasis, cardiomyopathies, and endothelial nitroglycerin tolerance.

Nonlimiting examples of lung diseases the antibody compositions and methods are used for ameliorating, or reducing the symptoms of, or treating, or preventing include, acute pneumonia, pulmonary fibrosis, interstitial pneumonia, pulmonary hypertension, chronic obstructive pulmonary disease (COPD), chronic bronchitis, pulmonary emphysema, asthma, refractory asthma, systemic inflammatory response syndrome (SIRS), lung injury acute (ALI), acute respiratory distress syndrome (ARDS), sarcoidosis, chronic idiopathic pulmonary thromboembolism, diffuse panbronchiolitis, cystic fibrosis, allergic alveolitis, lung cancer, obesity hypoventilation syndrome, alveolar hypoventilation syndrome and chronic transplant rejection pulmonary. Particularly important diseases are pulmonary fibrosis, interstitial pneumonia, pulmonary hypertension, asthma, COPD and SIRS.

Nonlimiting examples of cancers the antibody compositions and methods are used for ameliorating, or reducing the symptoms of, or treating, or preventing include cancers of the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus, or malignant neoplasm, carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Nonlimiting examples of neurological diseases the antibody compositions and methods are used for ameliorating, or reducing the symptoms of, or treating, or preventing include Alzheimer's disease, Parkinson's disease, Huntington's disease, Pick's disease, Kufs disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

Nonlimiting examples of metabolic diseases the antibody compositions and methods are used for ameliorating, or reducing the symptoms of, or treating, or preventing include metabolic syndrome, diabetes (type 1 diabetes, type 2 diabetes, gestational diabetes, etc.), impaired glucose tolerance, obesity, diabetic neuropathy, diabetic retinopathy, diabetic nephropathy, dyslipidemia Diseases (hypertriglyceridemia, hypercholesterolemia, hypoHDLemia, postprandial hyperlipidemia, etc.), hypertension, hypertriglyceridemia, severe hypertriglyceridemia, hypercholesterolemia, familial, elevated cholesterol caused by a genetic condition, fatty liver disease, nonalcoholic fatty liver disease (NFLD), nonalcoholic steatohepatitis (NASH), dyslipidemia, mixed dyslipidemia, atherosclerosis, and coronary heart disease.

The anti-IL-4RA proteins, antibodies or antibody fragments, are optionally administered in combination with one or more active agents including other analgesic agents. Such active agents include analgesic, anti-histamine, antipyretic, anti-inflammatory, antibiotic, antiviral, and anti-cytokine agents. Active agents include agonists, antagonists, and modulators of TNF-α, IL-2, IL-4, IL-6, IL-10, IL-12, IL-13, IL-18, IFN-α, IFN-γ, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors. Active agents also include, without limitation, 2-arylpropionic acids, aceclofenac, acemetacin, acetylsalicylic acid (Aspirin), alclofenac, alminoprofen, amoxiprin, ampyrone, arylalkanoic acids, azapropazone, benorylate/benorilate, benoxaprofen, bromfenac, carprofen, celecoxib, choline magnesium salicylate, clofezone, COX-2 inhibitors, dexibuprofen, dexketoprofen, diclofenac, diflunisal, droxicam, ethenzamide, etodolac, etoricoxib, faislamine, fenamic acids, fenbufen, fenoprofen, flufenamic acid, flunoxaprofen, flurbiprofen, ibuprofen, ibuproxam, indometacin, indoprofen, kebuzone, ketoprofen, ketorolac, lomoxicam, loxoprofen, lumiracoxib, magnesium salicylate, meclofenamic acid, mefenamic acid, meloxicam, metamizole, methyl salicylate, mofebutazone, nabumetone, naproxen, n-arylanthranilic acids, nerve growth factor (NGF), oxametacin, oxaprozin, oxicams, oxyphenbutazone, parecoxib, phenazone, phenylbutazone, phenylbutazone, piroxicam, pirprofen, profens, proglumetacin, pyrazolidine derivatives, rofecoxib, salicyl salicylate, salicylamide, salicylates, sulfinpyrazone, sulindac, suprofen, tenoxicam, tiaprofenic acid, tolfenamic acid, tolmetin, and valdecoxib.

An anti-histamine can be any compound that opposes the action of histamine or its release from cells (e.g., mast cells). Anti-histamines include but are not limited to acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratidine, methscopolamine, mizolastine, norastemizole, phenindamine, promethazine, pyrilamine, terfenadine, and tranilast.

Antibiotics include but are not limited to amikacin, aminoglycosides, amoxicillin, ampicillin, ansamycins, arsphenamine, azithromycin, azlocillin, aztreonam, bacitracin, carbacephem, carbapenems, carbenicillin, cefaclor, cefadroxil, cefalexin, cefalothin, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalosporins, chloramphenicol, cilastatin, ciprofloxacin, clarithromycin, clindamycin, cloxacillin, colistin, co-trimoxazole, dalfopristin, demeclocycline, dicloxacillin, dirithromycin, doripenem, doxycycline, enoxacin, ertapenem, erythromycin, ethambutol, flucloxacillin, fosfomycin, furazolidone, fusidic acid, gatifloxacin, geldanamycin, gentamicin, glycopeptides, herbimycin, imipenem, isoniazid, kanamycin, levofloxacin, lincomycin, linezolid, lomefloxacin, loracarbef, macrolides, mafenide, meropenem, meticillin, metronidazole, mezlocillin, minocycline, monobactams, moxifloxacin, mupirocin, nafcillin, neomycin, netilmicin, nitrofurantoin, norfloxacin, ofloxacin, oxacillin, oxytetracycline, paromomycin, penicillin, penicillins, piperacillin, platensimycin, polymyxin B, polypeptides, prontosil, pyrazinamide, quinolones, quinupristin, rifampicin, rifampin, roxithromycin, spectinomycin, streptomycin, sulfacetamide, sulfamethizole, sulfanilimide, sulfasalazine, sulfisoxazole, sulfonamides, teicoplanin, telithromycin, tetracycline, tetracyclines, ticarcillin, tinidazole, tobramycin, trimethoprim, trimethoprim-sulfamethoxazole, troleandomycin, trovafloxacin, and vancomycin.

Active agents also include aldosterone, beclometasone, betamethasone, corticosteroids, cortisol, cortisone acetate, deoxycorticosterone acetate, dexamethasone, fludrocortisone acetate, glucocorticoids, hydrocortisone, methylprednisolone, prednisolone, prednisone, steroids, and triamcinolone. Any suitable combination of these active agents is also contemplated.

### Formulations and Methods of Administration

For in vivo use, a therapeutic agent as described herein is generally incorporated into a pharmaceutical composition prior to administration. Within such compositions, one or more therapeutic compounds as described herein are present as active ingredient(s) (i.e., are present at levels sufficient to provide a statistically significant effect on the symptoms of cystic fibrosis, as measured using a representative assay). A pharmaceutical composition comprises one or more such compounds in combination with any pharmaceutically acceptable carrier(s) known to those skilled in the art to be suitable for the particular mode of administration. In addition, other pharmaceutically active ingredients (including other therapeutic agents) may, but need not, be present within the composition.

The antibodies of the present invention can be formulated according to standard methods (see, for example, Remington's Pharmaceutical Science, latest edition, Mark Publishing Company, Easton, U.S.A.), and may comprise pharmaceutically acceptable carriers and/or additives. The present invention relates to compositions (including reagents and pharmaceuticals) comprising the antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, antibodies of the present invention may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for making sustained-release drugs are known, and these can be applied for the antibodies of the present invention (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

The term “therapeutically effective amount,” in reference to treating a disease state/condition, refers to an amount of a compound either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat,” “treating” and “treatment” as used herein include administering a compound prior to the onset of clinical symptoms of a disease state/condition so as to prevent any symptom, as well as administering a compound after the onset of clinical symptoms of a disease state/condition so as to reduce or eliminate any symptom, aspect or characteristic of the disease state/condition. Such treating need not be absolute to be useful.

In certain embodiments, the present therapeutic agent may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds of the present invention can be determined by comparing their in vitro activity, and in vivo activity in animal models. In certain embodiments, a useful dose is from about 0.1 mg/kg to about 5 mg/kg or from about 0.5 mg/kg to about 2 mg/kg. Methods for the extrapolation of effective dosages in humans and animals of different sizes are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 M. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Exemplary IL-4RA receptor (IL-4Rα) constructs useful for screening, identifying, and evaluating anti-IL-4RA antibodies that block receptor binding include the following:

i) Extracellular domain (ECD) of the canine
IL-4 receptor α-V5 epitope-AviTag-His Tag
(SEQ ID NO: 1)
GSVKVLHEPSCFSDYISTSVCQWKMDHPTNCSAELRLSYQLDEMG
SENHTCVPENREDSVCVCSMPIDDAVEADVYQLDLWAGQQLLWSG
SFQPSKHVKPRTPGNLTVHPNISHTWLLMWTNPYPTENHLHSELT
YMVNVSNDNDPEDFKVYNVTYMGPTLRLAASTLKSGASYSARVRA
WAQTYNSTWSDWSPSTTWLNYYEPWEQHLGKPIPNPLLGLDSTGL
NDIFEAQKIEWHEHHHHHHHH
ii) Extracellular domain (ECD) of the canine
IL-4 receptor α-AviTag-His Tag
(SEQ ID NO: 2)
GSVKVLHEPSCFSDYISTSVCQWKMDHPTNCSAELRLSYQLDEMG
SENHTCVPENREDSVCVCSMPIDDAVEADVYQLDLWAGQQLLWSG
SFQPSKHVKPRTPGNLTVHPNISHTWLLMWTNPYPTENHLHSELT
YMVNVSNDNDPEDFKVYNVTYMGPTLRLAASTLKSGASYSARVRA
WAQTYNSTWSDWSPSTTWLNYYEPWEQHLGLNDIFEAQKIEWHEH
HHHHHHH

Without limitation, and subject to sequence variation disclosed herein, binding proteins of the invention may comprise one or more CDRs or variable domains of the following Table 1.

TABLE 1
Variable Domain Sequences

| SEQ | | |
|---|---|---|
| ID | | |
| NO: | Clone | |
| 5 | TB640_002 VH | EVQLVESGGDLVKPGGSLRLSCVASGFTFRDYAMTWVRQAPGKGLQWVSS |
| | | ISGSGGNTYYADAVKGRFTISRDNAKNTLYLQMNSLRAEDTAVYYCAKDR |
| | | LSITIRPRYYGLDVWGQGTLVTVSS |
| 6 | TB640_002 VL | DIVMTQTPLSLSVSPGETASISCRSSQSLLYSIGYNYLDWYLQKPGQSPQ |
| | | GLIYLGSKRASGVPDRFSGSGSGTDFTLRISRVEANDTGVYYCMQALQTP |
| | | YTFGQGTKLEIK |
| 7 | TB640_005 | EVQLVESGGDLVKPGGSLRLSCVASGFTFRDYAMTWVRQAPGKGLQWVSL |
| | H chain | ISGSGGKTYYADAVKGRFTISRDNAKNTLYLQMNSLRAEDTAVYYCAVDR |
| | | LTKTIRPQYYGLWVWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEMLGGPS |
| | | VFIFPPKPKDTLLIARTPEVTCVVVDLDPEDPEVQISWFVDGKQMQTAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICAVMHEALHNHYT |
| | | QESLSHSPG |
| 8 | TB640_005 | DIVMTQTPLSLSVSPGETASISCRSSQSLLYSIGYNYLDWYLQKPGQSPQ |
| | L chain | GLIYLGSKRASGVPDRFSGSGSGTDFTLRISRVEANDTGVYYCMQGLQSP |
| | | LTFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 9 | TB640_275 | EVQLVESGGDLVKPGGSLRLSCVASGFTFRDYAMDWVRQAPGKGLQWLSL |
| | H chain | ISGSGGKTYYADAVKGRFTISRDNAKNTLYLQMNSLRAEDTAVYYCAVDR |
| | | LTKTIRPQYYGLWVWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEMLGGPS |
| | | VFIFPPKPKDTLLIARTPEVTCVVVDLDPEDPEVQISWFVDGKQMQTAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICAVMHEALHNHYT |
| | | QESLSHSPG |
| 10 | TB640_275 | DIVMTQTPLSLSVSPGETASISCRSSQSLLYSIGYNYLDWYMQKPGQSPQ |
| | L chain | GLIYLGSKRASGVSDRFSGSGSGADFTLKISRVEADDAGVYYCMQGLQSP |
| | | LTFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 11 | FJP07H09-H | EVQLVESGGDLVKPGGSLRLSCVASGFTFRDYAMDWVRQAPGKGLQWLSL |
| | | ISGSGGKTYYADAVKGRFTISRDNAKNTLYLQMNSLRAEDTAVYYCAVDR |
| | | LTKTIRPQYYGLWSWGQGTLVTVSSASTTAPSVFPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEAAGGPS |
| | | VFIFPPKPKDTLLIARTPEVTCVVVDLDPEDPEVQISWFVDGKQMQLAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICYVMHEALHNHYT |
| | | QESLSHSPG |
| 12 | FJP06D01-H | EVQLVESGGDLVKPGGSLRLSCVASGFPFRDYAMDWVRQAPGKGLQWLSL |
| | | ISGSGGKTYYADAVKGRFTISRDNAKNTLYLQMNSLRAEDTAVYYCAVDR |
| | | LTKTIRPQYYGLWVWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEAAGGPS |
| | | VFIFPPKPKDTLLIARTPEVTCVVVDLDPEDPEVQISWFVDGKQMQLAKT |
| | | QPREEQFNGTYRVVSVLPIGHQDWLKGKQFTCKVNNKALPSPIERTISKA |
| | | RGQAHQPSVYVLPPSREELSKNTVSLTCLIKDEFPPDIDVEWQSNGQQEP |
| | | ESKYRTTPPQLDEDGSYFLYSKLSVDKSRWQRGDTFICYVMHEALHNHYT |
| | | QESLSHSPG |
| 13 | FJP05A03-H | EVQLVESGGDLVKPGGSLRLSCVASGLPFRDYAMAWVRQAPGKGLQWLSL |
| | | ISGSGGKTYYADAVKGRFTISRDNAKNTLYLQMNSLRAEDTAVYYCAVDR |
| | | LTKTIRPQYYGLWVWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEAAGGPS |
| | | VFIFPPKPKDTLLIARTPEVTCVVVDLDPEDPEVQISWFVDGKQMQLAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICYVMHEALHNHYT |
| | | QESLSHSPG |
| 14 | Fjmix_PS-H | EVQLVESGGDLVKPGGSLRLSCVASGFPFRDYAMDWVRQAPGKGLQWLSL |
| | | ISGSGGKTYYADAVKGRFTISRDNAKNTLYLQMNSLRAEDTAVYYCAVDR |
| | | LTKTIRPQYYGLWSWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEAAGGPS |
| | | VFIFPPKPKDTLLIARTPEVTCVVVDLDPEDPEVQISWFVDGKQMQLAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICYVMHEALHNHYT |
| | | QESLSHSPG |
| 15 | Gfmix_PTH-H | EVQLVESGGDLVKPGGSLRLSCVASGFPFRDYAMTWVRQAPGKGLQWLSL |
| | | ISGSGGKTYYADAVKGRFTISRDNAKNTLYLQMNSLRAEDTAVYYCAVDR |
| | | LTKTIRPQYYGLWHWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEAAGGPS |
| | | VFIFPPKPKDTLLIARTPEVTCVVVDLDPEDPEVQISWFVDGKQMQLAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICYVMHEALHNHYT |
| | | QESLSHSPG |
| 16 | 21188-H | EVQLVESGGDLVKPGGSLRLSCVASGFTFRDYAMTWVRQAPGKGLQWLSL |
| | | ISGSGGKTYYADAVKGRFTISRDNAKNTLYLQMNSLRAEDTAVYYCAVDR |
| | | LTKTIRPQYYGLWHWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEAAGGPS |
| | | VFIFPPKPKDTLLIARTPEVTCVVVDLDPEDPEVQISWFVDGKQMQLAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICYVMHEALHNHYT |
| | | QESLSHSPG |
| 17 | FJP10A09-L | DIVMTQTPLSLSVSPGETASISCRSSQSLTYSIGYNYLDWYMQKPGQSPQ |
| | | GLIYLGSKRASGVSDRESGSGSGADFTLKISRVEADDAGVYYCMQGLHSP |
| | | LSFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 18 | FJP10F04-L | DIVMTQTPLSLSVSPGETASISCRSSKSLLYSIGYNYLDWYMQKPGQSPQ |
| | | GLIYLGSKRASGVSDRFSGSGSGADFTLKISRVEADDAGVYYCMQGLHSP |
| | | LTFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 19 | GFmix_THS-L | DIVMTQTPLSLSVSPGETASISCRSSQSLTYSIGYNYLDWYMQKPGQSPQ |
| | | GLIYLGSKRASGVSDRESGSGSGADFTLKISRVEADDAGVYYCMQGLHSP |
| | | LGFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 20 | GFmix2_KTHG-L | DIVMTQTPLSLSVSPGETASISCRSSKSLTYSIGYNYLDWYMQKPGQSPQ |
| | | GLIYLGSKRASGVSDRESGSGSGADFTLKISRVEADDAGVYYCMQGLHSP |
| | | LGFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 21 | GFmix3_KTHS-L | DIVMTQTPLSLSVSPGETASISCRSSKSLTYSIGYNYLDWYMQKPGQSPQ |
| | | GLIYLGSKRASGVSDRFSGSGSGADFTLKISRVEADDAGVYYCMQGLHSP |
| | | LSFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 22 | 21188-L | DIVMTQTPLSLSVSPGETASISCRSSQSLTYSIGYNYLDWYMQKPGQSPQ |
| | | GLIYLGSKRASGVSDRFSGSGSGADFTLKISRVEADDAGVYYCMQGLQSP |
| | | LGFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 23 | FJ2246MP07H09 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWSWGQGTLVTVSS |
| 24 | FJ2246MP07A09 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGMWSWGQGTLVTVSS |
| 25 | FJ2246MP06H07 | EVQLVESGGDLVKPGGSLRLSCVASGFTFRDYAMTWVRQAPGKGLQWVSL |
| | | INGTGGKTYYADAVKGRFTISRDNAKNTLYLQMNSLRAEDTAVYYCAVDR |
| | | LTKTIRPQYYGLWAWGQGTLVTVSS |
| 26 | FJ2246MP06D01 | EVQLVESGGDLVKPGGSLRLSCVASGFPERDYAMTWVRQAPGKGLQWVSL |
| | | VNGSGSKTYYADAVKGRFTISRDNAKNTLYLQMNSLRAEDTAVYYCAVDR |
| | | LTKTIRPQYYGLWVWGQGTLVTVSS |
| 27 | FJ2246MP07A10 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTVRPQYYGLWSWGQGTLVTVSS |
| 28 | FJ2246MP05F12 | EVQLVESGGDLVKPGGSLRLSCVASGFPERDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 29 | FJ2246MP10A09 | DIVMTQTPLSLSVSPGETASISCRSSQSLLYSIGYNYLDWYLQKPGQSPQ |
| | | GLIYLGSKRASGVPDRESGSGSGTDFTLRISRVEAEDTGVYYCMQGLHSP |
| | | LSFGQGTKLEIK |
| 30 | FJ2246MP06G08 | EVQLVESGGDLVKPGGSLRLSCVASGFTFRDYAMTWVRQAPGKGLQWVSL |
| | | INGNGSKTYYADAVKGRFTISRDNAKNTLYLQMNSLRAEDTAVYYCAVDR |
| | | LTKTIRPQYYGLWVWGQGTLVTVSS |
| 31 | FJ2246MP10F04 | DIVMTQTPLSLSVSPGETASISCRSSKSLLYSIGYNYLDWYLQKPGQSPQ |
| | | GLIYLGSKRASGVPDRFSGSGSGTDFTLRISRVEADDTGVYYCMQGLHSP |
| | | LTFGQGTKLEIK |
| 32 | FJ2246MP10B12 | DIVMTQTPLSLSVSPGETASISCRSSQSLLYSIGYNYLDWYLQKPGQSPQ |
| | | GLIYLGSKRASGVPDRESGSGNGTDFTLRISRVEAEDTGVYYCMQGLHSP |
| | | LSFGQGTKLEIK |
| 33 | FJ2246MP06A04 | EVQLVESGGDLVKPGGSLRLSCVASGFTFRDYAMTWVRQAPGKGLQWVSL |
| | | INGNGGKTYYADAVKGRFTISRDNAKNTLYLQMNSLRAEDTAVYYCAVDR |
| | | LTKTIRPQYYGLWVWGQGTLVTVSS |
| 34 | FJ2246MP06E12 | EVQLVESGGDLVKPGGSLRLSCVASGFTFRDYAMTWVRQAPGKGLQWVSL |
| | | INGNGGKTYYADAVKGRFTISRDNAKNTLYLQMNSLRAEDTAVYYCAVDR |
| | | LTKTIRPQYYGLWVWGQGTLVTVSS |
| 35 | FJ2246MP10H11 | DIVMTQTPLSLSVSPGETASISCRSSQSLLYSIGYNYLDWYLQKPGQSPQ |
| | | GLIYLGSKRASGVPDRESGSGSGTDFTLRISRVEANDTGVYYCMQGLHSP |
| | | LSFGQGTKLEIK |
| 36 | FJ2246MP06A06 | EVQLVESGGDLVKPGGSLRLSCVASGFTFRDYAMTWVRQAPGKGLQWVSL |
| | | INGNGGKTYYADAVKGRFTISRDNAKNTLYLQMNSLRAEDTAVYYCAVDR |
| | | LTKTIRPQYYGLWVWGQGTLVTVSS |
| 37 | FJ2246MP06G07 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 38 | FJ2246MP06B01 | EVQLVESGGDLVKPGGSLRLSCVASGFTFRDYAMTWVRQAPGKGLQWVSL |
| | | INGNGGKTYYADAVKGRFTISRDNAKNTLYLQMNSLRAEDTAVYYCAVDR |
| | | LTKTIRPQYYGLWVWGQGTLVTVSS |
| 39 | FJ2246MP06D12 | EVQLVESGGDLVKPGGSLRLSCVASGFTFRDYAMTWVRQAPGKGLQWVSL |
| | | INGNSGKTYYADAVKGRFTISRDNAKNTLYLQMNSLRAEDTAVYYCAVDR |
| | | LTKTIRPQYYGLWVWGQGTLVTVSS |
| 40 | FJ2246MP06A09 | EVQLVESGGDLVKPGGSLRLSCVASGFTFRDYAMTWVRQAPGKGLQWVSL |
| | | INGNGSKTYYADAVKGRFTISRDNAKNTLYLQMNSLRAEDTAVYYCAVDR |
| | | LTKTIRPQYYGLWVWGQGTLVTVSS |
| 41 | FJ2246MP06H01 | EVQLVESGGDLVKPGGSLRLSCVASGFTFRDYAMTWVRQAPGKGLQWVSL |
| | | INGDASKTYYADAVKGRFTISRDNAKNTLYLQMNSLRAEDTAVYYCAVDR |
| | | LTKTIRPQYYGLWVWGQGTLVTVSS |
| 42 | FJ2246MP06G02 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 43 | FJ2246MP10G07 | DIVMTQTPLSLSVSPGETASISCRSSQSLLYSIGYNYLDWYLQKPGQSPQ |
| | | GLIYLGSKRASGVPDRFSGSGSGTDFTLRISRVEADDTGVYYCMQGLHSP |
| | | LTFGQGTKLEIK |
| 44 | FJ2246MP06F01 | EVQLVESGGDLVKPGGSLRLSCVASGFTFRDYAMTWVRQAPGKGLQWVSL |
| | | INGSGTKTYYADAVKGRFTISRDNAKNTLYLQMNSLRAEDTAVYYCAVDR |
| | | LTKTIRPQYYGLWVWGQGTLVTVSS |
| 45 | FJ2246MP06H04 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 46 | FJ2246MP06B04 | EVQLVESGGDLVKPGGSLRLSCVASGFTFRDYAMTWVRQAPGKGLQWVSL |
| | | INGNGGKTYYADAVKGRFTISRDNAKNTLYLQMNSLRAEDTAVYYCAVDR |
| | | LTKTIRPQYYGLWVWGQGTLVTVSS |
| 47 | FJ2246MP06A11 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 48 | FJ2246MP06H05 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 49 | FJ2246MP06A05 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 50 | FJ2246MP06E01 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 51 | FJ2246MP06B12 | EVQLVESGGDLVKPGGSLRLSCVASGFTFRDYAMTWVRQAPGKGLQWVSL |
| | | INGSGGKTYYADAVKGRETISRDNAKNTLYLQMNSLRAEDTAVYYCAVDR |
| | | LTKTIRPQYYGLWVWGQGTLVTVSS |
| 52 | FJ2246MP06C01 | EVQLVESGGDLVKPGGSLRLSCVASGFTERDYAMTWVRQAPGKGLQWVSL |
| | | INGNGGKTYYADAVKGRFTISRDNAKNTLYLQMNSLRAEDTAVYYCAVDR |
| | | LTKTIRPQYYGLWVWGQGTLVTVSS |
| 53 | FJ2246MP06A12 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 54 | FJ2246MP05A03 | EVQLVESGGDLVKPGGSLRLSCVASGLPERDYAMAWVRQAPGKGLQWVSL |
| | | ISGSGGKTYYADAVKGRFTISRDNAKNTLYLQMNSLRAEDTAVYYCAVDR |
| | | LTKTIRPQYYGLWVWGQGTLVTVSS |
| 55 | FJ2246MP06H10 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 56 | FJ2246MP10B07 | DIVMTQTPLSLSVSPGETASISCRSSQSLLYSIGYNYLDWYLQKPGQSPQ |
| | | GLIYLGSKRASGVPDRFSGSGSGTDFTLRISRVEAEDTGVYYCMQGLHSP |
| | | LTFGQGTKLEIK |
| 57 | FJ2246MP06H11 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 58 | FJ2246MP06A08 | EVQLVESGGDLVKPGGSLRLSCVASGFTFRDYAMTWVRQAPGKGLQWVSL |
| | | INGSGGKTYYADAVKGRETISRDNAKNTLYLQMNSLRAEDTAVYYCAVDR |
| | | LTKTIRPQYYGLWVWGQGTLVTVSS |
| 59 | FJ2246MP06C02 | EVQLVESGGDLVKPGGSLRLSCVASGFTFRDYAMTWVRQAPGKGLQWVSL |
| | | INGDGSKVYYADAVKGRFTISRDNAKNTLYLQMNSLRAEDTAVYYCAVDR |
| | | LTKTIRPQYYGLWVWGQGTLVTVSS |
| 60 | FJ2246MP10H03 | DIVMTQTPLSLSVSPGETASISCRSSQSLLYSIGYNYLDWYLQKPGQSPQ |
| | | GLIYLGSKRASGVPDRESGSGSGTDFTLRISRVEAEDTGVYYCMQGLRSP |
| | | LSFGQGTKLEIK |
| 61 | FJ2246MP10A10 | DIVMTQTPLSLSVSPGETASISCRSSQSLLYSIGYNYLDWYLQKPGQSPQ |
| | | GLIYLGSKRASGVPDRFSGSGSGTDFTLRISRVEAEDTGVYYCMQGLHSP |
| | | LTFGQGTKLEIK |
| 62 | FJ2246MP10B02 | DIVMTQTPLSLSVSPGETASISCRSSQSLLYSIGYNYLDWYLQKPGQSPQ |
| | | GLIYLGSKRASGVPDRFSGSGSGTDFTLRISRVEANDTGVYYCMQGLHSP |
| | | LTFGQGTKLEIK |
| 63 | FJ2246MP06B09 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 64 | FJ2246MP06A02 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 65 | FJ2246MP06B11 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 66 | FJ2246MP06D11 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 67 | FJ2246MP06C10 | EVQLVESGGDLVKPGGSLRLSCVASGFTFRDYAMTWVRQAPGKGLQWVSL |
| | | INGSGGKTYYADAVKGRFTISRDNAKNTLYLQMNSLRAEDTAVYYCAVDR |
| | | LTKTIRPQYYGLWVWGQGTLVTVSS |
| 68 | FJ2246MP10G04 | DIVMTQTPLSLSVSPGETASISCRSSQSLLYSIGYNYLDWYLQKPGQSPQ |
| | | GLIYLGSKRASGVPDRESGSGSGTDETLRISRVEAEDTGVYYCMQGLQSP |
| | | LSFGQGTKLEIK |
| 69 | FJ2246MP07E01 | EVQLVESGGDLVKPGGSLRLSCVASGFTFRDYAMTWVRQAPGKGLQWVSL |
| | | ISGSGGKTYYADAVKGRFTISRDNAKNTLYLQMNSLRAEDTAVYYCAVDR |
| | | LTKTIRPQYYGLWSWGQGTLVTVSS |
| 70 | FJ2246MP10D01 | DIVMTQTPLSLSVSPGETASISCRSSQSLLYSIGYNYLDWYLQKPGQSPQ |
| | | GLIYLGSKRASGVPDRESGSGSGTDFTLRISRVEADDTGVYYCMQGLQSP |
| | | LSFGQGTKLEIK |
| 71 | FJ2246MP10E12 | DIVMTQTPLSLSVSPGETASISCRSSQSLLYSIGYNYLDWYLQKPGQSPQ |
| | | GLIYLGSKRASGVPDRFSGSGSGTDFTLRISRVEAEDTGVYYCMQGLQSP |
| | | LSFGQGTKLEIK |
| 72 | FJ2246MP06A03 | EVQLVESGGDLVKPGGSLRLSCVASGFTFRDYAMTWVRQAPGKGLQWVSL |
| | | INGNGGKTYYADAVKGRFTISRDNAKNTLYLQMNSLRAEDTAVYYCAVDR |
| | | LTKTIRPQYYGLWVWGQGTLVTVSS |
| 73 | FJ2246MP05G01 | EVQLVESGGDLVKPGGSLRLSCVASGFPERDFAMTWVRQAPGKGLQWVSL |
| | | ISGSGGKTYYADAVKGRFTISRDNAKNTLYLQMNSLRAEDTAVYYCAVDR |
| | | LTKTIRPQYYGLWVWGQGTLVTVSS |
| 74 | FJ2246MP05A04 | EVQLVESGGDLVKPGGSLRLSCVASGLPERDYAMTWVRQAPGKGLQWVSL |
| | | ISGSGGKTYYADAVKGRFTISRDNAKNTLYLQMNSLRAEDTAVYYCAVDR |
| | | LTKTIRPQYYGLWVWGQGTLVTVSS |
| 75 | FJ2246MP06F11 | EVQLVESGGDLVKPGGSLRLSCVASGFTFRDYAMTWVRQAPGKGLQWVSL |
| | | INGDGSKTYYADAVKGRFTISRDNAKNTLYLQMNSLRAEDTAVYYCAVDR |
| | | LTKTIRPQYYGLWVWGQGTLVTVSS |
| 76 | TB640_057 | EVQLVESGGDLVKPGGSLRLSCVASGFTFRDYAMTWVRQAPGKGLQWVST |
| | heavy chain | ITGSGGNTYYADAVKGRFTISRDNAKNTLYLQMNSLRAEDTAVYYCAKDR |
| | | LSITIRPRYYGLDVWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEMLGGPS |
| | | VFIFPPKPKDTLLIARTPEVTCVVVDLDPEDPEVQISWFVDGKQMQTAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICAVMHEALHNHYT |
| | | QESLSHSPG |
| 77 | TB640_057 | DIVMTQTPLSLSVSPGETASISCRASQSLLYSIGYNYLDWYLQKPGQSPQ |
| | light chain | GLIYLGSKRASGVPDRESGSGSGTDFTLRISRVEANDTGVYYCMQAIQAP |
| | | YTFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |
| 78 | TB640_109 | EVQLVESGGDLVKPGGSLRLSCVASGFTFRDYAMTWVRQAPGKGLQWVST |
| | heavy chain | ISGSGGKTYYADAVKGRFTISRDNAKNTLYLQMNSLRAEDTAVYYCAVDR |
| | | LTKTIRPQYYGLSVWGQGTLVTVSSASTTAPSVEPLAPSCGSTSGSTVAL |
| | | ACLVSGYFPEPVTVSWNSGSLTSGVHTFPSVLQSSGLYSLSSMVTVPSSR |
| | | WPSETFTCNVAHPASKTKVDKPVPKRENGRVPRPPDCPKCPAPEMLGGPS |
| | | VFIFPPKPKDTLLIARTPEVTCVVVDLDPEDPEVQISWFVDGKQMQTAKT |
| | | QPREEQFNGTYRVVSVLPIGHQDWLKGKQFTCKVNNKALPSPIERTISKA |
| | | RGQAHQPSVYVLPPSREELSKNTVSLTCLIKDFFPPDIDVEWQSNGQQEP |
| | | ESKYRTTPPQLDEDGSYFLYSKLSVDKSRWQRGDTFICAVMHEALHNHYT |
| | | QESLSHSPG |
| 79 | TB640_109 | DIVMTQTPLSLSVSPGETASISCRASQSLLYSIGYNYLDWYLQKPGQSPQ |
| | light chain | GLIYLGSKRASGVPDRESGSGSGTDETLRISRVEANDTGVYYCMQALRDP |
| | | YTFGQGTKLEIKRNDAQPAVYLFQPSPDQLHTGSASVVCLLNSFYPKDIN |
| | | VKWKVDGVIQDTGIQESVTEQDKDSTYSLSSTLTMSSTEYLSHELYSCEI |
| | | THKSLPSTLIKSFQRSECQRVD |

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Binding of Dupilumab to Canine IL-4RA

Blocking the signaling of human IL-4 and IL-13 with an antibody directed against the human IL-4 receptor has been shown to be an effective therapeutic approach for treating atopic dermatitis and other skin disorders (Gooderham M J, Hong H C, Eshtiaghi P, Papp K A. 2018. Dupilumab: A review of its use in the treatment of atopic dermatitis. J Am Acad Dermatol. 78:S28-S36.) Dupilumab is an approved therapeutic antibody for the treatment of human atopic dermatitis, and it was previously demonstrated that dupilumab bound to canine IL-4 receptor a (IL-4RA) using an ELISA format (U.S. Pat. No. 11,091,556 B2).

An antibody was constructed containing the V_{H} variable domain of dupilumab and a human IgG1 constant region (instead of human IgG4 found in the therapeutic antibody) and the dupilumab Vκ light chain:

V_{H} (SEQ ID NO: 3):
EVQLVESGGGLEQPGGSLRLSCAGSGFTFRDYAMTWVRQAPGKGL
EWVSSISGSGGNTYYADSVKGRFTISRDNSKNTLYLQMNSLRAED
TAVYYCAKDRLSITIRPRYYGLDVWGQGTTVTVSSASTKGPSVFP
LAPSSKSTSGGTAALGCLVKDYFPEPVTVSWNSGALTSGVHTFPA
VLQSSGLYSLSSVVTVPSSSLGTQTYICNVNHKPSNTKVDKKVEP
KSCDKTHTCPPCPAPELLGGPSVFLFPPKPKDTLMISRTPEVTCV
VVDVSHEDPEVKFNWYVDGVEVHNAKTKPREEQYNSTYRVVSVLT
VLHQDWLNGKEYKCKVSNKALPAPIEKTISKAKGQPREPQVYTLP
PSRDELTKNQVSLTCLVKGFYPSDIAVEWESNGQPENNYKTTPPV
LDSDGSFFLYSKLTVDKSRWQQGNVFSCSVMHEALHNHYTQKSLS
Vκ (SEQ ID NO: 4):
DIVMTQSPLSLPVTPGEPASISCRSSQSLLYSIGYNYLDWYLQKS
GQSPQLLIYLGSNRASGVPDRFSGSGSGTDFTLKISRVEAEDVGF
YYCMQALQTPYTFGQGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC

To determine the affinity of dupilumab more accurately for canine IL-4RA, an SPR experiment was completed using a Biacore T200 instrument. In this experiment, a CM5 sensor chip (Cytiva, cat #BR100530) was used to immobilize anti-human Fc following the instructions of Human Antibody Capture Kit (Cytiva, cat #BR100839). Dupilumab (SEQ ID NOS:3 and 4) at 1 μg/mL was flowed at 10 μL/min for 60 sec to reach approximately 500 RU. The analyte was the extracellular domain of the canine IL-4 receptor (XP_022275636) was fused at the C-terminus with the V5 epitope which is derived from a small epitope (Pk) found on the P and V proteins of the paramyxovirus of the simian virus 5 family then fused to the AviTag™ sequence followed by a His tag utilized for purification (SEQ ID NO:1). This construct was expressed with the Expi293 method (ThermoFisher Scientific) and the canine IL-4RA-V5-Avi-His construct was purified from the conditioned Expi293 medium using a HisTrap column with a linear gradient from 20 to 500 mM imidazole in PBS, 500 mM NaCl, pH 7.4. This construct was further purified with size exclusion chromatography using HiLoad Superdex200 XK 50/70 to isolate the monomeric form and formulated in 50 mM sodium phosphate, 300 mM NaCl, pH 7.0. The running buffer was HBS-EP buffer (10 mM HEPES pH 7.4, 150 mM NaCl, 3 mM EDTA and 0.005% (v/v) P20 surfactant). The analyte was serial-diluted to 50 nM, 25 nM, 12.5 nM, 6.25 nM, 3.12 nM, 1.56 nM and 0.78 nM. All kinetic and steady state analyses were performed using BIAEVAL provided by BIACORE Inc.

The kinetics of dupilumab for canine IL-4RA are shown in Table 2.

TABLE 2
Affinity of dupilumab for canine IL-4RA

| | Kₒₙ (1/Ms) | K_{off} (1/s) | K_{D} (M) |
|---|---|---|---|
| Dupilumab | 1.089E+6 | 0.1434 | 1.3E−7 |

### Example 2: Caninization of Dupilumab Using a Phage Display Approach

Canine germline framework sequences for IGHV and IGKV were identified as IGHV3-9*01 (www.IMGT.org) and IGKV2-12*01 (www.IMGT.org) respectively for the library design. The J chain for the heavy framework 4 is canine IGHJ3*01 and the J chain for the light framework 4 is canine IGKJ3*01. Several framework positions were back-mutated to human sequences. These vernier positions are present in the original panning library and were later mutated to canine germline sequence. In the heavy chain these positions (by IMGT position) include D→T (H40) and L→V (H53). In the light chain, human germline back-mutated positions include M→L (L43), S→P (L72), and A→T (L85). Other positions within the light chain framework for the library were mutated from canine to human germline sequence, particularly K→R (L90), D→N(L97), and A→T (L99). The resulting starting sequences of the TB640_002 heavy and light chains, into which CDR mutations were introduced, are shown below. The underlined positions are those that are changed from the canine germline.

TB640_002 heavy chain variable domain
(SEQ ID NO: 5):
EVQLVESGGDLVKPGGSLRLSCVASGFTFRDYAMTWVRQAPGKGL
QWVSSISGSGGNTYYADAVKGRFTISRDNAKNTLYLQMNSLRAED
TAVYYCAKDRLSITIRPRYYGLDVWGQGTLVTVSS
TB640_002 light chain variable domain
(SEQ ID NO: 6):
DIVMTQTPLSLSVSPGETASISCRSSQSLLYSIGYNYLDWYLQKP
GQSPQGLIYLGSKRASGVPDRFSGSGSGTDFTLRISRVEANDTGV
YYCMQALQTPYTFGQGTKLEIK

Diversity was introduced into the CDRs at shown in Table 3.

TABLE 3
Amino acids introduced by CDR position
(sequential numbering from the amino terminal)

| | | | |
|---|---|---|---|
| HCDR1 | no changes | LCDR1 | L25: A, F, G, H, |
| | | | I, N, S, V, Y |
| | | | L26: S |
| | | | L27: Q |
| | | | L28: S |
| | | | L29: L |
| | | | L30: D, L, P, V |
| | | | L31: G, S, Y |
| | | | L32: E, P, R, S |
| | | | L33: I |
| | | | L34: G |
| | | | L35: Y |
| | | | L36: N |
| | | | L37: L |
| | | | L38: L, Q |
| HCDR2 | 50: A, D, E, F, G, H, | LCDR2 | no changes |
| | I, L, P, R, S, V, Y | | |
| | 51: F, I, L, S, T, V | | |
| | 52: G, I, R, S, T, V | | |
| | 53: A, D, E, G, H, I, | | |
| | L, S, T, V, Y | | |
| | 54: A, D, G, P, R, S, T | | |
| | 55: A, G, S | | |
| | 56: A, D, E, G, I, L, R, S, T, V | | |
| | 57: A, D, F, G, H, I, K, | | |
| | L, N, R, S, T, V, Y | | |
| | 58: A, I, K, P, R, S, T, V, Y | | |
| | 59: D, F, G, H, N, R, S, Y | | |
| HCDR3 | 98: D, K, L, M, T, V | LCDR3 | 94: E, G, M, R |
| | 99: D, G, S | | 95: H, Q, R |
| | 100: A, F, R | | 96: A, D, G, L, N, S, V |
| | 101: L | | 97: A, F, I, L, M, |
| | 102: A, D, E, F, G, | | P, Q, R, S, T, V |
| | L, M, S, T, W, Y | | 98: E, H, Q, R, T |
| | 103: D, E, I, K, L, M, V | | 99: A, D, E, F, G, |
| | 104: T | | H, I, L, N, P, |
| | 105: I | | R, S, T, V, Y |
| | 106: R | | 100: P |
| | 107: P | | 101: F, G, H, L, P, |
| | 108: D, E, L, Q, R, V | | R, S, W, Y |
| | 109: F, W, Y | | |
| | 110: K, R, T, Y | | |
| | 111: G | | |
| | 112: A, F, L, M, N W, Y | | |
| | 113: D, E, H, M, Q, S, W | | |

The final format for the library was single-chain variable fragments (scFv) cloned into a phagemid vector pADL22c. Assembled scFv-phagemid was transformed into TG1 electrocompetent E. coli by electroporation using standard procedures. A total of 6.6×10⁸ CFUs were generated from large scale electroporation. Sanger sequencing was performed on individual colonies from electroporation, 89% of which had open reading frames. Antibody phage selections were completed with the extracellular domain of canine IL-4RA for four rounds and with each round the stringency was increased by reducing the antigen concentration and increasing the number of washes. Specifically, Maxisorb 96-multi well plates were coated with 200 nM of canine IL-4RA for the first round, 100 nM for the second round and 50 pmol for the third and fourth rounds. The number of washes with PBS-tween 20 (0.1%) after the selection were 3 times for 5 minutes for the first round, 4 times for 5 minutes for the second round, 4 times for 5 minutes and 1 time for 15 minutes for the third round, and 4 times for 5 minutes and 2 times for 15 minutes for the fourth round. After the washing was completed for each round, the bound phage were eluted with trypsin. The output scFv clones from the third and fourth rounds were screened in a canine IL-4RA ELISA. The positive clones were sequenced and 130 unique clones were reformatted into IgGs and screened for binding to canine IL-4RA by SPR. The clones with the best affinity for canine IL-4RA were TB640_005 (heavy chain: SEQ ID NO:7; light chain: SEQ ID NO:8), TB640_057 (heavy chain: SEQ ID NO:76; light chain SEQ ID NO:77), and TB640_109 (heavy chain: SEQ ID NO:78; light chain SEQ ID NO:79). VH CDRs of the clones are compared with the TB640_002 in Table 4. VL CDRs of the clones are compared with the TB640_002 in Table 5.

TABLE 4
VH CDRs of IL-4RA-binding variants of TB640_002

| | | HCDR1 | HCDR2 | HCDR3 |
|---|---|---|---|---|
| SEQ | | | | 1 |
| ID | | 2 3 | 5 6 | 9 1 |
| NO | Clone | 7 5 | 0 6 | 8 4 |
| 5 | TB640_002 | FTFRDYAMT | SISGSGGNTYYADAVKG | KDRLSITIRPRYYGLDV |
| | | (SEQ ID | (SEQ ID NO: 93) | (SEQ ID NO: 94) |
| | | NO: 92) | | |
| 7 | TB640_005 | --------- | L------K--------- | V---TK----Q----W- |
| | | | (SEQ ID NO: 83) | (SEQ ID NO: 97) |
| 76 | TB640_057 | --------- | T-T-------------- | |
| | | | (SEQ ID NO: 95) | |
| 78 | TB640_109 | --------- | T------K--------- | V---TK----Q----S- |
| | | | (SEQ ID NO: 96) | (SEQ ID NO: 98) |

TABLE 5
VH CDRs of IL-4RA-binding variants of TB640_002

| SEQ | | | | | | | |
|---|---|---|---|---|---|---|---|
| ID | | | | | | | |
| NO | Clone | LCDR1 | | LCDR2 | | LCDR3 | |
| | | | | | | | 1 |
| | | 2 | 3 | 5 | 6 | 9 | 0 |
| | | 4 | 9 | 5 | 1 | 4 | 2 |

| | | | | |
|---|---|---|---|---|
| 6 | TB640-002 | RSSQSLLYSIGYNYLD | LGSKRAS | MQALQTPYT |
| | | (SEQ ID NO: 88) | (SEQ ID | (SEQ ID |
| | | | NO: 90) | NO: 100) |
| 8 | TB640_005 | - - - | - - - | - - - G - - - S-L- |
| | | | | (SEQ ID |
| | | | | NO: 101) |
| 77 | TB640_057 | -A - - - | - - - | - - - I-A - - - |
| | | (SEQ ID NO: 99) | | (SEQ ID |
| | | | | NO: 102) |
| 79 | TB640_109 | -A - - - | - - - | - - - RD - - - |
| | | (SEQ ID NO: 99) | | (SEQ ID |
| | | | | NO: 103) |

Affinities were determined by SPR on a Biacore T200. The antibodies were captured using a CM5 Series S chip amine coupled with an anti-dog H+L antibody (Jackson Immunoresearch). Antigen, canine IL-4RA (EQ ID NO:2), binding was then assessed at multiple concentrations starting at 100 nM using PBSP+ running buffer (Cytiva) with a flow rate of 30 μL/min. The canine IL-4RA was the extracellular domain of the canine IL-4 receptor (XP_022275636) fused at the C-terminus with the AviTag™ sequence followed by a His tag utilized for purification. This construct was expressed in CHO cells and purified with HisTrap FF chromatography followed by 25/600 Superdex chromatography as a polishing step. The length of the association time was 120 s and the dissociation time was run for 600 s. The chip surface was regenerated with 10 mM glycine. Double reference-subtracted sensorgrams were fitted to a 1:1 binding model using Biacore T200 Evaluation software and the kinetics of binding are shown below in Table 6.

TABLE 6
Affinity of three antibody clones for canine IL-4RA

| Clone | ka (1/Ms) | kd (1/s) | KD (M) | Rmax (RU) |
|---|---|---|---|---|
| TB640_005 | 1.80E+5 | 4.84E−4 | 2.72E−9 | 55 |
| TB640_057 | 1.94E+5 | 1.07E−3 | 5.54E−9 | 57 |
| TB640_109 | 2.25E+5 | 1.36E−3 | 6.06E−9 | 98 |

To remove the back mutations and non-germline residues, the TB640_005 CDRs (IMGT designation) were grafted onto IGHV3-9*01 completely canine germline for the heavy chain and IGKV2-12*01 canine germline for the light chain. This clone is referred to as TB640_275 (heavy chain: SEQ ID NO:7; light chain: SEQ ID NO:8) and the same method for determining the affinity for TB640_005 was used and the kinetics of binding are shown below in Table 7.

TABLE 7
Affinity of TB640_275 for canine IL-4RA

| Clone | ka (1/Ms) | kd (1/s) | KD (M) | Rmax (RU) |
|---|---|---|---|---|
| TB640_275 | 2.87E+5 | 4.87E−04 | 1.71E−09 | 63 |

### Example 3: Affinity Maturation of Clone TB640_005 Using Site-Specific Mutagenesis of Each CDR Position

In this affinity maturation approach, the heavy variable domain and the CH1 domain of caninized clone TB640_005 (SEQ ID NO:7, aa 1-221) was subcloned in the GenScript FASEBA plasmid. At the C-terminus of the heavy chain (VH-CH1) is the SASA (single-domain antibody against serum albumin) tag (see, e.g. US 2013/0129727A1) which has low pM affinity for albumin) and further downstream is a His-tag for purification. The light chain (SEQ ID NO:8) was subcloned into a proprietary E. coli expression vector. Both the heavy chain and light chain had the PelB (pectate lyase B) signal peptide at the N-terminus to facilitate secretion of the Fab when expressed in TG1 E. coli. The expression of the variable domains was regulated by the Lac promoter.

A variant library was generated for each CDR position in the heavy and light chains using the GenScript proprietary Precision Mutant Library (PML), which utilizes semiconductor-based oligonucleotide synthesis technology. The CDRs were defined using a combination of Kabat and IMGT methodology and the mutated residues selected for each CDR are shown below in Table 8. The residue numbers for the CDRs are shown in parentheses.

TABLE 8
TB640_005 CDR residues mutated
in libraries

| | VH | VL |
|---|---|---|
| CDR1 | GFTFRDYAMT | RSSQSLLYSIGYNYLD |
| | (26-35) | (24-39) |
| | (SEQ ID NO: 104) | (SEQ ID NO: 88) |
| CDR2 | LISGSGGKTYYAD | LGSKRAS |
| | AVKG (50-66) | (55-61) |
| | (SEQ ID NO: 83) | (SEQ ID NO: 90) |
| CDR3 | DRLTKTIRPQYYG | MQGLQSPLT |
| | LWV (99-114) | (94-102) |
| | (SEQ ID NO: 86) | (SEQ ID NO: 101) |

Forty-four PML clones were selected from each library for expression in E. coli in 96 deep-well plates by inoculating into 2YT medium and inducing with 0.2 mM IPTG overnight at room temperature. The Fab secreted in the medium was analyzed for binding activity by completing an ELISA. In this ELISA, plates were coated with 10 μg/ml of BSA overnight at 4° C., washed 3× with 0.1% tween 20 in PBS, pH 7.4 (PBST), blocked non-specific interactions with 3% non-fat dry milk in PBS (phosphate-buffered saline, pH 7.4) at 37° C. for 1 hour, washed 3× with PBST, added crude Fab supernatant (diluted 1:1 with PBST) incubated at 37° C. for 1 hour, washed 3× with PBST, added canine IL-4RA (SEQ ID NO:1) incubated at 37° C. for 1 hour, washed 3× with PBST, added horseradish peroxidase (HRP) conjugated anti-His tag antibody (His tag present on canine IL-4RA) incubated at room temperature for 45 minutes, washed 3× with PBST and detected the HRP conjugate by incubating with TMB substrate for 10 minutes at room temperature and measured absorbance at 450 nm. The top 73 clones with an apparent increase in affinity as measured by ELISA were sequenced to detect the variant in the CDR. The 39 unique clones were confirmed by an off-rate screening assay in an SPR assay performed on a Biacore T200. For the SPR analyses, bovine serum albumin (BSA) was immobilized to CM5 sensor chip. The sensor chip surface was activated with 50 mmol/L H-Hydroxysuccinimide and 200 mmol/L 1-ethyl-3-(3-dimethylaminopropyl) carbodiimide hydrochloride for 420 s. Afterwards, BSA diluted in 10 mM sodium acetate, pH 4.5 was injected. After the amine coupling reaction, the remaining active coupling sites on the chip surface were blocked with 1 mM ethanolamine hydrochloride. The selected Fab-SASA variants in conditioned medium were captured on the BSA-coated chips. The running buffer was HBS-EP (10 mM HEPES 500 mM NaCl, 3 mM EDTA, 0.05% Tween 20, pH 7.4). After equilibration, canine IL-4RA was injected for 120 seconds (association phase) followed by the injection of running buffer for 420 sec (dissociation phase). The off-rates of the Fab-SASA clones were obtained from fitting the experimental data locally to a 1:1 interaction model using the Biacore T200 evaluation software. The Fab variants were ranked by their dissociation rate constants (off-rates, kd) in Table 9.

TABLE 9
Rank ordering of the TB640_005 Fab variants based on dissociation constants

| | | | | | | Ratio | |
|---|---|---|---|---|---|---|---|
| | | | | | | WT-kd/ | Selected |
| Sequence | Ka | Kd | KD | Rmax | Chi² | clone− | for |
| Analysis | (1/ms) | (1/s) | (M) | (RU) | (RU²) | kd | Library |
| VH-S52N | 6.62E+04 | 1.12E−04 | 1.83E−09 | 34.70 | 1.34E+00 | 7.88 | X |
| VL-G96P | 1.09E+05 | 3.58E−04 | 3.27E−09 | 14.00 | 1.25E−01 | 2.66 | X |
| VL-T102G | 7.85E+04 | 4.51E−04 | 5.74E−09 | 18.60 | 3.25E−01 | 2.11 | X |
| VL-Q95N | 1.16E+05 | 4.80E−04 | 4.15E−09 | 29.70 | 6.95E−01 | 1.99 | |
| VH-L112M | 3.53E+04 | 4.83E−04 | 1.37E−08 | 48.50 | 5.03E−01 | 1.97 | |
| VL-T102S | 7.44E+04 | 4.92E−04 | 6.61E−09 | 38.50 | 1.33E+00 | 1.94 | X |
| VL-T102N | 8.01E+04 | 5.48E−04 | 6.83E−09 | 28.50 | 7.58E−01 | 1.74 | X |
| VL-T102L | 8.60E+04 | 5.62E−04 | 6.53E−09 | 20.60 | 4.20E−01 | 1.70 | X |
| VH-V114H | 1.08E+05 | 5.89E−04 | 5.47E−09 | 54.30 | 2.80E+00 | 1.62 | X |
| VL-L30T | 5.22E+04 | 6.18E−04 | 1.18E−08 | 28.40 | 1.34E+00 | 1.54 | X |
| VH-L101K | 8.78E+04 | 6.23E−04 | 7.10E−09 | 54.40 | 2.56E+00 | 1.53 | X |
| VH-K103R | 8.92E+04 | 6.36E−04 | 7.13E−09 | 31.70 | 1.10E+00 | 1.50 | |
| VH-V114G | 1.85E+05 | 6.47E−04 | 3.50E−09 | 59.90 | 9.78E−01 | 1.47 | |
| VL-L55M | 8.39E+04 | 6.69E−04 | 7.97E−09 | 35.50 | 1.31E+00 | 1.42 | |
| VH-V114Q | 9.41E+04 | 6.74E−04 | 7.16E−09 | 52.30 | 2.87E+00 | 1.41 | |
| VL-S57H | 8.26E+04 | 6.77E−04 | 8.20E−09 | 50.40 | 2.31E+00 | 1.41 | X |
| VH-L101M | 9.23E+04 | 7.13E−04 | 7.73E−09 | 49.40 | 2.73E+00 | 1.34 | |
| VH-V114K | 1.20E+05 | 7.17E−04 | 5.97E−09 | 66.10 | 3.42E+00 | 1.33 | |
| VL-S57N | 8.18E+04 | 7.20E−04 | 8.80E−09 | 47.90 | 2.20E+00 | 1.32 | |
| VH-T28N | 6.92E+04 | 7.80E−04 | 1.13E−08 | 46.40 | 2.41E+00 | 1.22 | |
| VH-T28P | 8.78E+04 | 7.86E−04 | 8.95E−09 | 31.50 | 1.14E+00 | 1.21 | |
| VL-A60M | 1.88E+05 | 8.50E−04 | 4.52E−09 | 41.40 | 6.23E−01 | 1.12 | |
| VL-Q27E | 6.53E+04 | 8.73E−04 | 1.34E−08 | 37.10 | 1.87E+00 | 1.09 | |
| VL-Y35M | 1.12E+05 | 9.32E−04 | 8.34E−09 | 34.10 | 9.86E−01 | 1.02 | |
| VH-K103I | 5.19E+04 | 9.67E−04 | 1.86E−08 | 17.40 | 4.56E−01 | 0.99 | |
| VL-A60L | 1.80E+05 | 9.74E−04 | 5.41E−09 | 35.50 | 2.57E−01 | 0.98 | |
| VL-Q27H | 6.57E+04 | 9.79E−04 | 1.49E−08 | 36.00 | 1.56E+00 | 0.97 | |
| VL-Q27L | 6.42E+04 | 9.96E−04 | 1.55E−08 | 32.00 | 1.24E+00 | 0.96 | |
| VL-I33V | 7.98E+04 | 1.01E−03 | 1.27E−08 | 32.70 | 1.07E+00 | 0.94 | |
| VL-S57T | 7.04E+04 | 1.05E−03 | 1.50E−08 | 52.60 | 2.86E+00 | 0.91 | |
| VL-Q27T | 6.97E+04 | 1.11E−03 | 1.59E−08 | 38.20 | 1.73E+00 | 0.86 | |
| VH-L50M | 1.41E+05 | 1.20E−03 | 8.50E−09 | 38.10 | 1.53E+00 | 0.79 | |
| VH-Y110L | 2.02E+05 | 1.35E−03 | 6.65E−09 | 42.50 | 7.93E−01 | 0.71 | |
| VL-I33P | 1.09E+05 | 1.37E−03 | 1.25E−08 | 32.80 | 6.66E−01 | 0.70 | |
| VH-Y110R | 1.59E+05 | 1.53E−03 | 9.60E−09 | 55.10 | 1.28E+00 | 0.62 | |
| VL-I33A | 7.59E+04 | 1.53E−03 | 2.01E−08 | 32.70 | 1.17E+00 | 0.62 | |
| VL-L30A | 6.86E+04 | 1.87E−03 | 2.73E−08 | 42.20 | 2.55E+00 | 0.51 | |
| VH-L50F | 1.28E+05 | 1.93E−03 | 1.50E−08 | 39.90 | 1.34E+00 | 0.49 | |
| VL-L101N | 3.28E+05 | 3.21E−03 | 9.76E−09 | 41.70 | 4.81E−01 | 0.30 | |
| WT | 7.31E+04 | 9.52E−04 | 1.30E−08 | 33.50 | 1.19E+00 | 1.00 | |
| WT | 7.17E+04 | 9.53E−04 | 1.33E−08 | 32.00 | 1.07E+00 | 1.00 | |
| WT | 7.24E+04 | 9.53E−04 | 1.32E−08 | 32.75 | 1.13E+00 | 1.00 | |

The distribution of single CDR mutations in the top TB640_005 Fab variants is shown in Table 10 and Table 11, below.

TABLE 10
VH CDR mutations in top-ranked Fab
variants of TB640_005

| | HCDR1 | HCDR2 | HCDR3 |
|---|---|---|---|
| | | | 1 |
| | 2 3 | 5 6 | 9 1 |
| | 6 5 | 0 6 | 9 4 |
| TB640_005 | GFTFRDYAMT | LISGSGGKTYYADAVKG | DRLIKTIRPQYYGLWV |
| | (SEQ ID | (SEQ ID NO: 83) | (SEQ ID NO: 86) |
| | NO: 104) | | |
| S52N | ---------- | --N-------------- | ---------------- |
| | | (SEQ ID NO: 108) | |
| L112M | ---------- | ----------------- | -------------M-- |
| | | | (SEQ ID NO: 111) |
| V114H | ---------- | ----------------- | ---------------H |
| | | | (SEQ ID NO: 112) |
| L101K | ---------- | ----------------- | --K------------- |
| | | | (SEQ ID NO: 113) |
| K103R | ---------- | ----------------- | ----R----------- |
| | | | (SEQ ID NO: 114) |
| V114G | ---------- | ----------------- | ---------------G |
| | | | (SEQ ID NO: 115) |
| V114Q | ---------- | ----------------- | ---------------Q |
| | | | (SEQ ID NO: 116) |
| L101M | ---------- | ----------------- | --M------------- |
| | | | (SEQ ID NO: 117) |
| V114K | ---------- | ----------------- | ---------------K |
| | | | (SEQ ID NO: 118) |
| T28N | --N------- | ----------------- | ---------------- |
| | (SEQ ID | | |
| | NO: 106) | | |
| T28P | --P------- | ----------------- | ---------------- |
| | (SEQ ID | | |
| | NO: 107) | | |
| K103I | ---------- | ----------------- | ----I----------- |
| | | | (SEQ ID NO: 119) |
| L50M | ---------- | M---------------- | |
| | | (SEQ ID NO: 109) | |
| Y110L | ---------- | ----------------- | ------------L--- |
| | | | (SEQ ID NO: 120) |
| Y110R | ---------- | ----------------- | -----------R---- |
| | | | (SEQ ID NO: 121) |
| L50F | ---------- | F---------------- | ---------------- |
| | | (SEQ ID NO: 110) | |

TABLE 10
VK CDR mutations in top-ranked Fab variants
of TB640_005

| | LCDR1 | LCDR2 | LCDR3 |
|---|---|---|---|
| | | | 1 |
| | 2 3 | 5 6 | 9 0 |
| | 4 9 | 5 1 | 4 2 |
| TB640_005 | RSSQSLLYSIGYNYLD | LGSKRAS | MQGLQSPLT |
| | (SEQ ID NO: 88) | (SEQ ID | (SEQ ID |
| | | NO: 90) | NO: 101) |
| G96P | ---------------- | ------- | --P------ |
| | | | (SEQ ID |
| | | | NO: 138) |
| T102G | ---------------- | ------- | --------G |
| | | | (SEQ ID |
| | | | NO: 139) |
| Q95N | ---------------- | ------- | -N------- |
| | | | (SEQ ID |
| | | | NO: 140) |
| T102S | ---------------- | ------- | --------S |
| | | | (SEQ ID |
| | | | NO: 141) |
| T102N | ---------------- | ------- | --------N |
| | | | (SEQ ID |
| | | | NO: 142) |
| T102L | ---------------- | ------- | --------L |
| | | | (SEQ ID |
| | | | NO: 143) |
| L30T | -------T-------- | ------- | --------- |
| | (SEQ ID NO: 122) | | |
| L55M | ---------------- | M------ | --------- |
| | | (SEQ ID | |
| | | NO: 133) | |
| S57H | ---------------- | --H---- | --------- |
| | | (SEQ ID | |
| | | NO: 134) | |
| S57N | ---------------- | --N---- | --------- |
| | | (SEQ ID | |
| | | NO: 135) | |
| A60M | ---------------- | -----M- | --------- |
| | | (SEQ ID | |
| | | NO: 136) | |
| Q27E | ---E------------ | ------- | --------- |
| | (SEQ ID NO: 123) | | |
| Y35M | -----------M---- | ------- | --------- |
| | (SEQ ID NO: 124) | | |
| Y35L | -----------L---- | ------- | --------- |
| | (SEQ ID NO: 125) | | |
| A60L | ---------------- | -----L- | --------- |
| | | (SEQ ID | |
| | | NO: 410) | |
| Q27H | ---H------------ | ------- | --------- |
| | (SEQ ID NO: 126) | | |
| Q27L | ---L------------ | ------- | --------- |
| | (SEQ ID NO: 127) | | |
| I33V | ---------V------ | ------- | --------- |
| | (SEQ ID NO: 128) | | |
| S57T | ---------------- | --T---- | --------- |
| | | (SEQ ID | |
| | | NO: 137) | |
| Q27T | ---T------------ | ------- | --------- |
| | (SEQ ID NO: 129) | | |
| I33P | ---------P------ | ------- | --------- |
| | (SEQ ID NO: 130) | | |
| I33A | ---------A------ | ------- | --------- |
| | (SEQ ID NO: 131) | | |
| L30A | -----A---------- | ------- | --------- |
| | (SEQ ID NO: 132) | | |
| L101N | ---------------- | ------- | -------N- |
| | | | (SEQ ID |
| | | | NO: 144) |

### Example 4: Combinatorial Library of Selected TB640_005 Variants

Fab variant amino acids from the above tables were selected for a combinatorial Fab library (Table 12).

TABLE 12
Selected Fab variants for the combinatorial library

| | VH | | | VL | | | |
|---|---|---|---|---|---|---|---|
| | 52 | 101 | 114 | 30 | 57 | 96 | 102 |
| WT | S | L | V | L | S | G | T |
| Variant | N | K | H | T | H | P | L, N, G, S |

The combinatorial library was constructed in the same Fab-SASA construct described earlier. The theoretical diversity of the combinatorial library is 2×2×2×2×2×2×5=320 and the size of the constructed library was 5.6×10⁷ CFU (colony forming units). The library in-frame rate was 85% for the heavy chain and 98% for the light chain. The diversity was evaluated by DNA sequencing and the results are shown in the Table 13 and Table 14 below.

TABLE 13
Variant Frequency of VH Fragment

| | Analysis | S52N | L101K | V114H |
|---|---|---|---|---|
| VH | 41 | N(13), S(28) | L(31), K(10) | V(37), H(4) |

TABLE 14
Variant Frequency of VL Fragment

| | Analysis | L30T | S57H | G96P | T102S/N/G/L |
|---|---|---|---|---|---|
| VL | 47 | L(25), T(22) | S(25), H(22) | G(43), P(4) | T(10), S(13), N(2), |
| | | | | | G(18), L(4) |

From the combinatorial library, 368 clones were randomly selected binding and expressed binding by ELISA. The top 32 clones were sequenced and the 23 unique clones were evaluated by SPR shown in Table 15

TABLE 11
SPR analysis of 23 clones from combinatorial library

| | | | | Rmax | VH | | | VL | | | |
|---|---|---|---|---|---|---|---|---|---|---|---|
| Sequence ID | ka (1/Ms) | kd (1/s) | KD (M) | (RU) | S52 | L101 | V114 | L30 | S57 | G96 | T102 |
| AHF21170 | 1.47E+05 | 1.38E−04 | 9.40E−10 | 32.4 | N | — | — | T | — | — | — |
| AHF21171 | 2.85E+05 | 1.34E−04 | 4.70E−10 | 49.7 | N | — | H | — | H | — | S |
| AHF21172 | 2.26E+05 | 1.68E−04 | 7.40E−10 | 39.7 | N | — | — | — | H | — | — |
| AHF21173 | 1.60E+05 | 9.69E−05 | 6.06E−10 | 35.3 | N | K | — | T | H | — | S |
| AHF21174 | 1.84E+05 | 9.58E−05 | 5.21E−10 | 34.7 | N | — | — | T | H | — | S |
| AHF21176 | 2.95E+05 | 9.11E−05 | 3.09E−10 | 47.6 | N | K | H | T | H | — | G |
| AHF21177 | 1.70E+05 | 9.52E−05 | 5.61E−10 | 32.4 | N | — | — | T | — | — | G |
| AHF21178 | 1.78E+05 | 8.27E−05 | 4.66E−10 | 30.2 | N | — | — | T | — | — | S |
| AHF21180 | 2.19E+05 | 5.68E−05 | 2.59E−10 | 33.1 | N | K | H | T | — | P | - |
| AHF21182 | 2.86E+05 | 1.26E−04 | 4.42E−10 | 38 | N | — | H | T | H | — | L |
| AHF21184 | 2.87E+05 | 5.43E−04 | 1.89E−09 | 46.1 | — | — | H | — | — | — | S |
| AHF21186 | 3.06E+05 | 1.48E−04 | 4.85E−10 | 44.2 | N | — | H | — | H | — | — |
| AHF21187 | 1.72E+05 | 1.32E−04 | 7.69E−10 | 27.1 | N | K | — | T | H | — | G |
| AHF21188 | 3.25E+05 | 2.78E−04 | 8.57E−10 | 43.4 | — | — | H | T | — | — | G |
| AHF21190 | 2.23E+05 | 1.52E−04 | 6.80E−10 | 33.3 | N | — | — | — | H | — | S |
| AHF21191 | 2.59E+05 | 9.48E−05 | 3.66E−10 | 43.5 | N | — | H | T | — | — | G |
| AHF21192 | 2.39E+05 | 1.61E−04 | 6.73E−10 | 43.8 | N | K | — | T | — | — | - |
| AHF21194 | 2.12E+05 | 1.33E−04 | 6.27E−10 | 42.3 | N | — | — | — | — | — | S |
| AHF21195 | 1.93E+05 | 1.92E−04 | 9.92E−10 | 45.8 | N | K | — | — | — | — | S |
| AHF21197 | 2.14E+05 | 1.26E−04 | 5.88E−10 | 30.6 | N | K | — | T | — | — | — |
| AHF21199 | 2.07E+05 | 8.18E−05 | 3.95E−10 | 27.9 | N | — | — | T | H | — | G |
| AHF21200 | 1.46E+05 | 1.27E−04 | 8.75E−10 | 33.4 | N | K | — | T | — | P | S |
| AHF21201 | 3.44E+05 | 1.51E−04 | 4.40E−10 | 34.8 | N | — | H | — | H | — | G |
| Wild-type | 1.91E+05 | 1.16E−03 | 6.07E−09 | 29.1 | — | — | — | — | — | — | — |

### Example 5: Affinity Maturation of Clone TB640_005 Using Phage Display Libraries of Mutated Individual CDRs

In this strategy, affinity maturation phage display libraries were generated by mutagenesis of the CDRs. Individual CDR libraries were generated by introducing mutations to have three to four mutations per CDR. Up to four rounds of selections were completed with each of the six individual CDR libraries using biotinylated canine IL-4RA. The periplasmic extracts of the phage from the later rounds of selections were assayed in an off-rate assay using SPR. The positive clones were sequenced to identify the variants responsible for the slower off-rate.

The residues in each of the CDR regions of TB640_005 subjected to mutagenesis are underlined in Table 16.

TABLE 12
CDR and adjacent residues mutated
in library

| | VH | VL |
|---|---|---|
| CDR1 | GFTFRDYAMT | RSSQSLLYSIGYN |
| | (26-35) | YLDWYL (24-42) |
| | (SEQ ID NO: 104) | (SEQ ID NO: 145) |
| CDR2 | LISGSGGKTYYA | LGSKRAS |
| | DAVKG (50-66) | (55-61) |
| | (SEQ ID NO: 83) | (SEQ ID NO: 90) |
| CDR3 | VDRLTKTIRPQY | MQGLQSPLT |
| | YGLWV (98-114) | (94-102) |
| | (SEQ ID NO: 97) | (SEQ ID NO: 101) |

The CDR variant libraries were generated by PCR (polymerase chain reaction) using a large number of proprietary oligonucleotides and cloned into either the VL-WT_VFJB129 for the light chain or VH-WT_VFJB129 phagemids. The PCR was a three-step amplification at three different annealing temperatures (50° C., 55° C. and 60° C.) using the overlapping oligonucleotides. The PCR reactions were agarose gel-purified, digested with compatible restriction enzymes and subcloned into digested phagemid vectors for heavy or light chains. The ligation mixtures were transformed into ECC TG1 cells (Lucigen, Cat number 60502-2). Phage from the library bacterial cultures were rescued and precipitated. The library sizes are shown in Table 17.

TABLE 13
Individual library sizes

| | | Library size (Colony |
|---|---|---|
| Library name | Library ID | Forming Units) |
| IVX-04005AFM HCDR1 | FL3439 | 6.3E+08 |
| IVX-04005AFM_HCDR2 | FL3440 | 7.5E+09 |
| IVX-04005AFM_HCDR3 | FL3441 | 5.5E+09 |
| IVX-04005AFM LCDR1 | FL3442 | 5.4E+09 |
| IVX-04005AFM_LCDR2 | FL3443 | 1.8E+08 |
| IVX-04005AFM_LCDR3 | FL3444 | 1.6E+09 |

Approximately sixty clones from each library were sequenced to determine the quality of the libraries. Table 18 below describes the percentage of full-length clones and percentage of wild-type sequences.

TABLE 14
Percentage of full-length clones and wild-type sequences

| | | | % | % | |
|---|---|---|---|---|---|
| | | | valid | valid | % wild- |
| | Library | Clones | VH | VL | type |
| Library Name | ID | sequenced | clones | clones | sequences |
| IVX-04005AFM_HCDR1 | FL3439 | 63 | 75% | | 3% |
| IVX-04005AFM_HCDR2 | FL3440 | 63 | 69% | | 3% |
| IVX-04005AFM_HCDR3 | FL3441 | 62 | 71% | | 9% |
| IVX-04005AFM_LCDR1 | FL3442 | 63 | | 61% | 3% |
| IVX-04005AFM_LCDR2 | FL3443 | 63 | | 62% | 0% |
| IVX-04005AFM_LCDR3 | FL3444 | 62 | | 72% | 0% |

Evaluating the clones sequenced for each library, the average number of mutations per chain was three in all the libraries except for IVX-04005AFM_HCDR1 and IVX-04005AFM_LCDR1 which had an average of four mutations. In approximately 50% of the clones there were spurious one to two mutations in the frameworks that were presumably PCR-induced.

In-solution selections were carried out for the heavy and light chain libraries. Dynabeads Streptavidin T1 magnetic beads were coated with biotinylated canine-IL-4RA (SEQ ID NO:1) as the antigen. The canine IL-4RA protein (SEQ ID NO:1) was biotinylated at the Avi-tag site using the BirA500-RT kit (Avidity).

A KingFisher Flex system was used to wash the beads using 2% nonfat dry milk in PBS. Trypsin at 1 mg/ml was used to elute the phage after each selection step. Table 19 below has the IL-4RA concentration conditions for the selection rounds of the heavy chain CDR libraries.

TABLE 15
IL-4RA concentration conditions for the heavy chain selection rounds.

| HCDR | | | | |
|---|---|---|---|---|
| libraries | Round 1 | Round 2 | Round 3 | Round 4 |
| FL3439 | 10 nM IL-4RA | 0.8 nM IL-4RA | 1 nM IL-4RA | 1 nM, 0.1 nM, |
| | | | | 0.01 nM IL-4RA |
| FL3440 | 10 nM IL-4RA | 0.8 nM IL-4RA | 1 nM IL-4RA | 1 nM, 0.1 nM, |
| | | | | 0.01 nM IL-4RA |
| FL3441 | 10 nM IL-4RA | 0.8 nM IL-4RA | 1 nM IL-4RA | 1 nM IL-4RA |

The wash conditions for each round were for round 1, 100×IL-4RA (concentration used in selection) for 2 hours; for round 2, 100×IL-4RA for 20 hours; for round 3, 100×IL-4RA for 48 hours and for round 4, 100×IL-4RA for 72 hours.

Table 20 below provides the IL-4RA concentration conditions for the selection rounds of the light chain CDR libraries.

TABLE 16
IL-4RA concentration conditions for the light chain selection rounds.

| HCDR | | | | |
|---|---|---|---|---|
| libraries | Round 1 | Round 2 | Round 3 | Round 4 |
| FL3442 | 10 nM | 0.8 nM IL-4RA | 1 nM IL-4RA | 1 nM, 0.1 nM, |
| | IL-4RA | | | 0.01 nM IL-4RA |
| FL3440 | 10 nM | 0.8 nM IL-4RA | 1 nM IL-4RA | 1 nM, 0.1 nM, |
| | IL-4RA | | | 0.01 nM IL-4RA |
| FL3441 | 10 nM | 0.8 nM IL-4RA | 1 nM IL-4RA | 1 nM, 0.1 |
| | IL-4RA | | | nM IL-4RA |

The wash conditions for each round of light chain selections were the same as for the heavy chain CDR libraries.

Ninety-two clones from the output of the round 4 for each CDR library were plated into 96-well plates and grown. Periplasmic extracts containing soluble Fabs were prepared and screened using SPR. For this method, canine IL-4RA was immobilized on a CM5 sensor chip (Cytiva; cat #29149603) using a standard amine coupling method at approximately 1000 RU immobilization levels. Periplasmic extracts were diluted 1:5 into 1×HBS-EP, pH 7.4 (Cytiva; cat #BR-1008-26) and injected for 2 minutes at 30 μl/min. The dissociation rate was assessed for 5 minutes. The instrument used for the analyses was a Biacore 8K. Data were analyzed using the multi-cycle kinetics predefined evaluation method of the Biacore Insight Evaluation software. Dissociation constants were calculated using the 1:1 dissociation Langmuir binding model. All libraries generated binders with a kd having an increase of 2-fold over the parental antibody with the exception of the LCDR1 and LCDR2 libraries which didn't yield any higher affinity binders. All of the binders were sequenced. Sequences of CDRs of binders recovered from the separate CDR libraries compared to TB640_500 are shown in Table 21 for VH CDRs and Table 22 for VL CDRs. Amino acid numbering is sequential from the VH or VL amino terminal.

TABLE 17
VH CDRs of IL-4RA-binding variants of TB640_500

| HCDR1 | HCDR2 | HCDR3 |
|---|---|---|
| | | 1 |
| 2 3 | 5 6 | 9 1 |
| 7 5 | 0 6 | 8 4 |
| FTFRDYAMT | LISGSGGKTYYADAVKG | VDRLTKTIRPQYYGLWV |
| (SEQ ID NO: 92) | (SEQ ID NO: 83) | (SEQ ID NO: 97) |
| -A-D----- | --N-DAS---------- | A---------------- |
| (SEQ ID NO: 146) | (SEQ ID NO: 195) | (SEQ ID NO: 221) |
| -A-G----- | --N-D-S---------- | I---------------- |
| (SEQ ID NO: 147) | (SEQ ID NO: 196) | (SEQ ID NO: 222) |
| -A------- | --N-D-S-V-------- | R--------------Q- |
| (SEQ ID NO: 148) | (SEQ ID NO: 197) | (SEQ ID NO: 223) |
| -A------V | --N-I------------ | S-----------M-M-- |
| (SEQ ID NO: 149) | (SEQ ID NO: 198) | (SEQ ID NO: 224) |
| -D-G----- | --N-N---I-------- | T----H----------S |
| (SEQ ID NO: 150) | (SEQ ID NO: 199) | (SEQ ID NO: 225) |
| -D------- | --N-N------------ | T---------------- |
| (SEQ ID NO: 151) | (SEQ ID NO: 200) | (SEQ ID NO: 226) |
| -D------V | --N-N--R--------- | T-------------M-- |
| (SEQ ID NO: 152) | (SEQ ID NO: 201) | (SEQ ID NO: 227) |
| -I------- | --N-N-S---------- | ---I--------T---- |
| (SEQ ID NO: 153) | (SEQ ID NO: 202) | (SEQ ID NO: 228) |
| -P-G----- | --N-NS----------- | ------M---------S |
| (SEQ ID NO: 154) | (SEQ ID NO: 203) | (SEQ ID NO: 229) |
| -P-N-F--- | --N-NS--V-------- | ----------A-Q---- |
| (SEQ ID NO: 155) | (SEQ ID NO: 204) | (SEQ ID NO: 230) |
| -P---F--- | --N---D---------- | ------------A---S |
| (SEQ ID NO: 156) | (SEQ ID NO: 205) | (SEQ ID NO: 231) |
| -P------S | --N---DR--------- | ------------A---- |
| (SEQ ID NO: 157) | (SEQ ID NO: 206) | (SEQ ID NO: 232) |
| -P------- | --N----G--------- | ------------E---L |
| (SEQ ID NO: 158) | (SEQ ID NO: 207) | (SEQ ID NO: 233) |
| -P------V | --N--D----------- | ------------E---- |
| (SEQ ID NO: 159) | (SEQ ID NO: 208) | (SEQ ID NO: 234) |
| -S-N----V | --N-------------D | ------------F---- |
| (SEQ ID NO: 160) | (SEQ ID NO: 108) | (SEQ ID NO: 235) |
| -S------- | --N-----V-------- | ------------L---- |
| (SEQ ID NO: 161) | (SEQ ID NO: 209) | (SEQ ID NO: 236) |
| ---G----- | --N---N---------- | ------------M---- |
| (SEQ ID NO: 162) | (SEQ ID NO: 210) | (SEQ ID NO: 237) |
| ---S----- | --N---T---------- | ------------N---- |
| (SEQ ID NO: 163) | (SEQ ID NO: 211) | (SEQ ID NO: 238) |
| ---S----V | --N-T------------ | ------------Q---- |
| (SEQ ID NO: 164) | (SEQ ID NO: 212) | (SEQ ID NO: 239) |
| IA------- | --------S-------- | ------------R-F-D |
| (SEQ ID NO: 165) | (SEQ ID NO: 213) | (SEQ ID NO: 240) |
| IA------V | --------S-------- | ------------S---D |
| (SEQ ID NO: 166) | (SEQ ID NO: 213) | (SEQ ID NO: 241) |
| IA-S----V | -------N--------- | ------------S---- |
| (SEQ ID NO: 167) | (SEQ ID NO: 214) | (SEQ ID NO: 242) |
| ID------V | ------T---------- | ------------T---- |
| (SEQ ID NO: 168) | (SEQ ID NO: 215) | (SEQ ID NO: 243) |
| IP-G----A | -VN---S---------- | ----------------A |
| (SEQ ID NO: 169) | (SEQ ID NO: 216) | (SEQ ID NO: 244) |
| IP-G----- | S-N-------------- | ----------------D |
| (SEQ ID NO: 170) | (SEQ ID NO: 217) | (SEQ ID NO: 245) |
| IP-G---- | S-N----SI-------- | ----------------F |
| (SEQ ID NO: 170) | (SEQ ID NO: 218) | (SEQ ID NO: 246) |
| IP-G----V | T-N-N------------ | ----------------H |
| (SEQ ID NO: 171) | (SEQ ID NO: 219) | (SEQ ID NO: 247) |
| IR-G----- | T-N---N---------- | ----------------S |
| (SEQ ID NO: 172) | (SEQ ID NO: 220) | (SEQ ID NO: 248) |
| IR------V | | --------------M-H |
| (SEQ ID NO: 173) | | (SEQ ID NO: 249) |
| IS-G----A | | --------------M-S |
| (SEQ ID NO: 174) | | (SEQ ID NO: 250) |
| IS------V | | -----------V-R--- |
| (SEQ ID NO: 175) | | (SEQ ID NO: 251) |
| I--G----- | | -------V--------S |
| (SEQ ID NO: 176) | | (SEQ ID NO: 252) |
| I-------- | | -------V--------- |
| (SEQ ID NO: 177) | | (SEQ ID NO: 253) |
| I-------V | | -----N------M---- |
| (SEQ ID NO: 178) | | (SEQ ID NO: 254) |
| I--S----V | | ---M------------D |
| (SEQ ID NO: 179) | | (SEQ ID NO: 255) |
| I-S-----V | | ---M------------S |
| (SEQ ID NO: 180) | | (SEQ ID NO: 256) |
| LA------V | | ---M------------- |
| (SEQ ID NO: 181) | | (SEQ ID NO: 257) |
| LD-G----- | | |
| (SEQ ID NO: 182) | | |
| LD------- | | |
| (SEQ ID NO: 194) | | |
| LD------V | | |
| (SEQ ID NO: 183) | | |
| LP-G----- | | |
| (SEQ ID NO: 184) | | |
| LP------A | | |
| (SEQ ID NO: 185) | | |
| LP------- | | |
| (SEQ ID NO: 186) | | |
| LR-G----V | | |
| (SEQ ID NO: 187) | | |
| LR------- | | |
| (SEQ ID NO: 188) | | |
| LS------V | | |
| (SEQ ID NO: 189) | | |
| L-------- | | |
| (SEQ ID NO: 190) | | |
| L--S----- | | |
| (SEQ ID NO: 191) | | |
| L--S----V | | |
| (SEQ ID NO: 192) | | |
| VA------- | | |
| (SEQ ID NO: 193) | | |

TABLE 18
VL CDRs of IL-4RA-binding variants of TB640_500

| LCDR1 | LCDR2 | LCDR3 |
|---|---|---|
| | | 1 |
| 2 3 | 5 6 | 9 0 |
| 4 9 | 5 1 | 4 2 |
| RSSQSLLYSIGYNYLD | LGSKRAS | MQGLQSPLT |
| (SEQ ID NO: 88) | (SEQ ID NO: 90) | (SEQ ID NO: 101) |
| ---G-V---S----- | A-N--Q- | ----F--P- |
| (SEQ ID NO: 258) | (SEQ ID NO: 303) | (SEQ ID NO: 335) |
| ---K--I-------V- | H-N--Q- | ----HN--- |
| (SEQ ID NO: 259) | (SEQ ID NO: 304) | (SEQ ID NO: 336) |
| ---K-----G------ | --D---A | ----HN-P- |
| (SEQ ID NO: 260) | (SEQ ID NO: 305) | (SEQ ID NO: 337) |
| ---K-------L--V- | --N---A | ----H---S |
| (SEQ ID NO: 261) | (SEQ ID NO: 306) | (SEQ ID NO: 338) |
| ---K----------I- | --N---P | ----H---- |
| (SEQ ID NO: 262) | (SEQ ID NO: 307) | (SEQ ID NO: 339) |
| ---K------------ | --N---- | ----H---V |
| (SEQ ID NO: 263) | (SEQ ID NO: 135) | (SEQ ID NO: 340) |
| ---K----------V- | --N---T | ----I---S |
| (SEQ ID NO: 264) | (SEQ ID NO: 308) | (SEQ ID NO: 341) |
| ---K-----T-R---- | --N--QA | ----I---- |
| (SEQ ID NO: 265) | (SEQ ID NO: 309) | (SEQ ID NO: 342) |
| ----K----------- | --N--Q- | --------S |
| (SEQ ID NO: 266) | (SEQ ID NO: 310) | (SEQ ID NO: 141) |
| ---K-----V------ | --N--S- | -------V- |
| (SEQ ID NO: 267) | (SEQ ID NO: 311) | (SEQ ID NO: 343) |
| ---K-----V----V- | --NR--A | -------Y- |
| (SEQ ID NO: 268) | (SEQ ID NO: 312) | (SEQ ID NO: 344) |
| ---K--S-------V- | --NR--- | ----R---S |
| (SEQ ID NO: 269) | (SEQ ID NO: 313) | (SEQ ID NO: 345) |
| ---K-V---A----V- | ----L-A | ----R---- |
| (SEQ ID NO: 270) | (SEQ ID NO: 314) | (SEQ ID NO: 346) |
| ------I--NR---- | ----L-- | ----R--V- |
| (SEQ ID NO: 271) | (SEQ ID NO: 315) | (SEQ ID NO: 347) |
| ---------A-T---- | ------A | ----S---- |
| (SEQ ID NO: 272) | (SEQ ID NO: 316) | (SEQ ID NO: 348) |
| ---------A----I- | ------G | ----T---- |
| (SEQ ID NO: 273) | (SEQ ID NO: 317) | (SEQ ID NO: 349) |
| --------------A- | ------P | --N-HF-P- |
| (SEQ ID NO: 131) | (SEQ ID NO: 318) | (SEQ ID NO: 350) |
| ---------A----V- | ------Q | --S----I- |
| (SEQ ID NO: 274) | (SEQ ID NO: 319) | (SEQ ID NO: 351) |
| ----------GD---- | ------R | --S-----S |
| (SEQ ID NO: 275) | (SEQ ID NO: 320) | (SEQ ID NO: 352) |
| ---------G-A---- | ------T | --S------ |
| (SEQ ID NO: 276) | (SEQ ID NO: 321) | (SEQ ID NO: 353) |
| ---------GL----- | -----QA | --S----Y- |
| (SEQ ID NO: 277) | (SEQ ID NO: 322) | (SEQ ID NO: 354) |
| -----------A---- | -----Q- | -------I- |
| (SEQ ID NO: 278) | (SEQ ID NO: 323) | (SEQ ID NO: 355) |
| -----------F--I- | -----QT | ----T--IV |
| (SEQ ID NO: 279) | (SEQ ID NO: 324) | (SEQ ID NO: 356) |
| -----------F--V- | -----SA | --------V |
| (SEQ ID NO: 280) | (SEQ ID NO: 325) | (SEQ ID NO: 357) |
| -----------N--I- | ----Y- | |
| (SEQ ID NO: 281) | (SEQ ID NO: 326) | |
| -----------S---- | ---N--- | |
| (SEQ ID NO: 282) | (SEQ ID NO: 327) | |
| ----------S--V- | --T---- | |
| (SEQ ID NO: 283) | (SEQ ID NO: 137) | |
| --------------I- | --T--QA | |
| (SEQ ID NO: 284) | (SEQ ID NO: 328) | |
| --------------V- | --Y---P | |
| (SEQ ID NO: 285) | (SEQ ID NO: 329) | |
| ---------NRT---- | --Y---- | |
| (SEQ ID NO: 286) | (SEQ ID NO: 330) | |
| ---------S-I---- | --Y---T | |
| (SEQ ID NO: 287) | (SEQ ID NO: 331) | |
| ---------S------ | -SN--SA | |
| (SEQ ID NO: 288) | (SEQ ID NO: 332) | |
| ---------S----V- | N-Y---A | |
| (SEQ ID NO: 289) | (SEQ ID NO: 333) | |
| ---------V----I- | Q--R--A | |
| (SEQ ID NO: 290) | (SEQ ID NO: 334) | |
| ------S-I------- | | |
| (SEQ ID NO: 291) | | |
| ------S--------- | | |
| (SEQ ID NO: 292) | | |
| ------S-------V- | | |
| (SEQ ID NO: 293) | | |
| -----V---A------ | | |
| (SEQ ID NO: 294) | | |
| -----V---G------ | | |
| (SEQ ID NO: 295) | | |
| -----V---T----V- | | |
| (SEQ ID NO: 296) | | |
| ---R-----V------ | | |
| (SEQ ID NO: 297) | | |
| ---T----G------- | | |
| (SEQ ID NO: 298) | | |
| ---T------------ | | |
| (SEQ ID NO: 129) | | |
| ---T----------V- | | |
| (SEQ ID NO: 299) | | |
| ---T--Q----K---- | | |
| (SEQ ID NO: 300) | | |
| ---T--S----R---- | | |
| (SEQ ID NO: 301) | | |
| ---TT----A----V- | | |
| (SEQ ID NO: 302) | | |

Binding affinities of the top clones are shown in Table 23 and sequences of complete VH or VL domains are identified. Of the top clones, CDRs of the top binding VH chains are shown in Table 24 and CDRs of the top binding VH chains are shown in Table 25. Full VH and VL variable domain sequences are provided in Sequence Table 1.

TABLE 19
Binding of the top TB640_005 variants

| SEQ | | | | | Improvement |
|---|---|---|---|---|---|
| ID | | | RU | | over |
| NO: | Clone ID | Library ID | max | kd (1/s) | parental |
| 23 | FJ2246MP07H09 | FL3441 | 509.4 | 5.23E−05 | 5.4 |
| 24 | FJ2246MP07A09 | FL3441 | 252.0 | 6.73E−05 | 4.2 |
| 25 | FJ2246MP06H07 | FL3440 | 568.0 | 7.50E−05 | 3.7 |
| 26 | FJ2246MP06D01 | FL3440 | 323.3 | 7.72E−05 | 3.6 |
| 27 | FJ2246MP07A10 | FL3441 | 614.3 | 8.18E−05 | 3.4 |
| 28 | FJ2246MP05F12 | FL3439 | 208.5 | 8.83E−05 | 3.2 |
| 29 | FJ2246MP10A09 | FL3444 | 436.8 | 8.91E−05 | 3.1 |
| 30 | FJ2246MP06G08 | FL3440 | 199.3 | 9.01E−05 | 3.1 |
| 31 | FJ2246MP10F04 | FL3444 | 291.5 | 9.16E−05 | 3.1 |
| 32 | FJ2246MP10B12 | FL3444 | 304.2 | 9.41E−05 | 3.0 |
| 33 | FJ2246MP06A04 | FL3440 | 187.5 | 9.59E−05 | 2.9 |
| 34 | FJ2246MP06E12 | FL3440 | 126.9 | 9.66E−05 | 2.9 |
| 35 | FJ2246MP10H11 | FL3444 | 261.0 | 9.90E−05 | 2.8 |
| 35 | FJ2246MP06A06 | FL3440 | 183.9 | 1.00E−04 | 2.8 |
| 37 | FJ2246MP06G07 | FL3440 | 181.4 | 1.00E−04 | 2.8 |
| 38 | FJ2246MP06B01 | FL3440 | 192.8 | 1.01E−04 | 2.8 |
| 39 | FJ2246MP06D12 | FL3440 | 153.6 | 1.02E−04 | 2.7 |
| 40 | FJ2246MP06A09 | FL3440 | 152.1 | 1.06E−04 | 2.6 |
| 41 | FJ2246MP06H01 | FL3440 | 225.4 | 1.06E−04 | 2.6 |
| 42 | FJ2246MP06G02 | FL3440 | 173.4 | 1.07E−04 | 2.6 |
| 43 | FJ2246MP10G07 | FL3444 | 372.5 | 1.11E−04 | 2.5 |
| 44 | FJ2246MP06F01 | FL3440 | 198.8 | 1.12E−04 | 2.5 |
| 45 | FJ2246MP06H04 | FL3440 | 193.6 | 1.12E−04 | 2.5 |
| 46 | FJ2246MP06B04 | FL3440 | 172.6 | 1.12E−04 | 2.5 |
| 47 | FJ2246MP06A11 | FL3440 | 180.0 | 1.15E−04 | 2.4 |
| 48 | FJ2246MP06H05 | FL3440 | 185.9 | 1.16E−04 | 2.4 |
| 49 | FJ2246MP06A05 | FL3440 | 185.6 | 1.18E−04 | 2.4 |
| 50 | FJ2246MP06E01 | FL3440 | 151.5 | 1.19E−04 | 2.4 |
| 51 | FJ2246MP06B12 | FL3440 | 148.1 | 1.21E−04 | 2.3 |
| 52 | FJ2246MP06C01 | FL3440 | 160.3 | 1.22E−04 | 2.3 |
| 53 | FJ2246MP06A12 | FL3440 | 134.2 | 1.24E−04 | 2.3 |
| 54 | FJ2246MP05A03 | FL3439 | 400.0 | 1.24E−04 | 2.3 |
| 55 | FJ2246MP06H10 | FL3440 | 139.8 | 1.24E−04 | 2.3 |
| 56 | FJ2246MP10B07 | FL3444 | 252.2 | 1.25E−04 | 2.2 |
| 57 | FJ2246MP06H11 | FL3440 | 162.0 | 1.25E−04 | 2.2 |
| 58 | FJ2246MP06A08 | FL3440 | 156.1 | 1.25E−04 | 2.2 |
| 59 | FJ2246MP06C02 | FL3440 | 272.2 | 1.26E−04 | 2.2 |
| 60 | FJ2246MP10H03 | FL3444 | 243.2 | 1.26E−04 | 2.2 |
| 61 | FJ2246MP10A10 | FL3444 | 368.6 | 1.26E−04 | 2.2 |
| 62 | FJ2246MP10B02 | FL3444 | 202.2 | 1.27E−04 | 2.2 |
| 63 | FJ2246MP06B09 | FL3440 | 160.9 | 1.27E−04 | 2.2 |
| 64 | FJ2246MP06A02 | FL3440 | 163.0 | 1.29E−04 | 2.2 |
| 65 | FJ2246MP06B11 | FL3440 | 123.8 | 1.31E−04 | 2.1 |
| 66 | FJ2246MP06D11 | FL3440 | 146.1 | 1.32E−04 | 2.1 |
| 67 | FJ2246MP06C10 | FL3440 | 135.4 | 1.33E−04 | 2.1 |
| 68 | FJ2246MP10G04 | FL3444 | 251.3 | 1.34E−04 | 2.1 |
| 69 | FJ2246MP07E01 | FL3441 | 423.4 | 1.35E−04 | 2.1 |
| 70 | FJ2246MP10D01 | FL3444 | 283.0 | 1.37E−04 | 2.0 |
| 71 | FJ2246MP10E12 | FL3444 | 251.0 | 1.39E−04 | 2.0 |
| 72 | FJ2246MP06A03 | FL3440 | 116.6 | 1.40E−04 | 2.0 |
| 73 | FJ2246MP05G01 | FL3439 | 343.6 | 1.42E−04 | 2.0 |
| 74 | FJ2246MP05A04 | FL3439 | 328.9 | 1.42E−04 | 2.0 |
| 75 | FJ2246MP06F11 | FL3440 | 195.9 | 1.43E−04 | 2.0 |
| | FJ2246MP06G06 | Parental Fab | 105.2 | 2.80E−04 | 1.0 |

HCDRs of top VH variants are shown in Table 24.

TABLE 20
HCDR sequences of top TB640_005 variants

| | | HCDR1 | HCDR2 | HCDR3 |
|---|---|---|---|---|
| SEQ | | | | 1 |
| ID | | 2 3 | 5 6 | 9 1 |
| NO: | Clone ID | 7 5 | 0 6 | 8 4 |
| 23 | FJ2246MP07H09 | FTERDYAMT | LINGSGGKTYYADAVKG | VDRLTKTIRPQYYGLWS |
| | | (SEQ ID | (SEQ ID NO: 108) | (SEQ ID NO: 248) |
| | | NO: 92) | | |
| 24 | FJ2246MP07A09 | FTERDYAMT | LINGSGGKTYYADAVKG | VDRLTKTIRPQYYGMWS |
| | | (SEQ ID | (SEQ ID NO: 108) | (SEQ ID NO: 250) |
| | | NO: 92) | | |
| 25 | FJ2246MP06H07 | FTERDYAMT | LINGTGGKTYYADAVKG | VDRLTKTIRPQYYGLWA |
| | | (SEQ ID | (SEQ ID NO: 212) | (SEQ ID NO: 244) |
| | | NO: 92) | | |
| 26 | FJ2246MP06D01 | FPFRDYAMT | LVNGSGSKTYYADAVKG | VDRLTKTIRPQYYGLWV |
| | | (SEQ ID | (SEQ ID NO: 216) | (SEQ ID NO: 97) |
| | | NO: 158) | | |
| 27 | FJ2246MP07A10 | FTFRDYAMT | LINGSGGKTYYADAVKG | VDRLTKTVRPQYYGLWS |
| | | (SEQ ID | (SEQ ID NO: 108) | (SEQ ID NO: 252) |
| | | NO: 92) | | |
| 28 | FJ2246MP05F12 | FPFRDYAMT | LINGSGGKTYYADAVKG | VDRLTKTIRPQYYGLWV |
| | | (SEQ ID | (SEQ ID NO: 108) | (SEQ ID NO: 97) |
| | | NO: 158) | | |
| 30 | FJ2246MP06G08 | FTERDYAMT | LINGNGSKTYYADAVKG | VDRLTKTIRPQYYGLWV |
| | | (SEQ ID | (SEQ ID NO: 202) | (SEQ ID NO: 97) |
| | | NO: 92) | | |
| 33 | FJ2246MP06A04 | FTERDYAMT | LINGNGGKTYYADAVKG | VDRLTKTIRPQYYGLWV |
| | | (SEQ ID | (SEQ ID NO: 200) | (SEQ ID NO: 97) |
| | | NO: 92) | | |
| 34 | FJ2246MP06E12 | FTFRDYAMT | LINGNGGKTYYADAVKG | VDRLTKTIRPQYYGLWV |
| | | (SEQ ID | (SEQ ID NO: 200) | (SEQ ID NO: 97) |
| | | NO: 92) | | |
| 36 | FJ2246MP06A06 | FTERDYAMT | LINGNGGKTYYADAVKG | VDRLTKTIRPQYYGLWV |
| | | (SEQ ID | (SEQ ID NO: 200) | (SEQ ID NO: 97) |
| | | NO: 92) | | |
| 37 | FJ2246MP06G07 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 38 | FJ2246MP06B01 | FTERDYAMT | LINGNGGKTYYADAVKG | VDRLTKTIRPQYYGLWV |
| | | (SEQ ID | (SEQ ID NO: 200) | (SEQ ID NO: 97) |
| | | NO: 92) | | |
| 39 | FJ2246MP06D12 | FTERDYAMT | LINGNSGKTYYADAVKG | VDRLTKTIRPQYYGLWV |
| | | (SEQ ID | (SEQ ID NO: 203) | (SEQ ID NO: 97) |
| | | NO: 92) | | |
| 40 | FJ2246MP06A09 | FTERDYAMT | LINGNGSKTYYADAVKG | VDRLTKTIRPQYYGLWV |
| | | (SEQ ID | (SEQ ID NO: 202) | (SEQ ID NO: 97) |
| | | NO: 92) | | |
| 41 | FJ2246MP06H01 | FTERDYAMT | LINGDASKTYYADAVKG | VDRLTKTIRPQYYGLWV |
| | | (SEQ ID | (SEQ ID NO: 195) | (SEQ ID NO: 97) |
| | | NO: 92) | | |
| 42 | FJ2246MP06G02 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 44 | FJ2246MP06F01 | FTERDYAMT | LINGSGTKTYYADAVKG | VDRLTKTIRPQYYGLWV |
| | | (SEQ ID | (SEQ ID NO: 211) | (SEQ ID NO: 97) |
| | | NO: 92) | | |
| 45 | FJ2246MP06H04 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 46 | FJ2246MP06B04 | FTERDYAMT | LINGNGGKTYYADAVKG | VDRLTKTIRPQYYGLWV |
| | | (SEQ ID | (SEQ ID NO: 200) | (SEQ ID NO: 97) |
| | | NO: 92) | | |
| 47 | FJ2246MP06A11 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 48 | FJ2246MP06H05 | FTFRDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 49 | FJ2246MP06A05 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 50 | FJ2246MP06E01 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 51 | FJ2246MP06B12 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 52 | FJ2246MP06C01 | FTERDYAMT | LINGNGGKTYYADAVKG | VDRLTKTIRPQYYGLWV |
| | | (SEQ ID | (SEQ ID NO: 200) | (SEQ ID NO: 97) |
| | | NO: 92) | | |
| 53 | FJ2246MP06A12 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 54 | FJ2246MP05A03 | LPFRDYAMA | LISGSGGKTYYADAVKG | VDRLTKTIRPQYYGLWV |
| | | (SEQ ID | (SEQ ID NO: 83) | (SEQ ID NO: 97) |
| | | NO: 185) | | |
| 55 | FJ2246MP06H10 | FTFRDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 57 | FJ2246MP06H11 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 58 | FJ2246MP06A08 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 59 | FJ2246MP06C02 | FTERDYAMT | LINGDGSKVYYADAVKG | VDRLTKTIRPQYYGLWV |
| | | (SEQ ID | (SEQ ID NO: 197) | (SEQ ID NO: 97) |
| | | NO: 92) | | |
| 63 | FJ2246MP06B09 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 64 | FJ2246MP06A02 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 65 | FJ2246MP06B11 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 66 | FJ2246MP06D11 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 67 | FJ2246MP06C10 | FTERDYAMT | LINGSGGKTYYADAVKG | VDRLTKTIRPQYYGLWV |
| | | (SEQ ID | (SEQ ID NO: 108) | (SEQ ID NO: 97) |
| | | NO: 92) | | |
| 69 | FJ2246MP07E01 | FTERDYAMT | LISGSGGKTYYADAVKG | VDRLTKTIRPQYYGLWS |
| | | (SEQ ID | (SEQ ID NO: 83) | (SEQ ID NO: 248) |
| | | NO: 92) | | |
| 72 | FJ2246MP06A03 | FTERDYAMT | LINGNGGKTYYADAVKG | VDRLTKTIRPQYYGLWV |
| | | (SEQ ID | (SEQ ID NO: 200) | (SEQ ID NO: 97) |
| | | NO: 92) | | |
| 73 | FJ2246MP05G01 | FPFRDFAMT | LISGSGGKTYYADAVKG | VDRLTKTIRPQYYGLWV |
| | | (SEQ ID | (SEQ ID NO: 83) | (SEQ ID NO: 97) |
| | | NO: 156) | | |
| 74 | FJ2246MP05A04 | LPERDYAMT | LISGSGGKTYYADAVKG | VDRLTKTIRPQYYGLWV |
| | | (SEQ ID | (SEQ ID NO: 83) | (SEQ ID NO: 97) |
| | | NO: 186) | | |
| 75 | FJ2246MP06F11 | FTERDYAMT | LINGDGSKTYYADAVKG | VDRLTKTIRPQYYGLWV |
| | | (SEQ ID | (SEQ ID NO: 196) | (SEQ ID NO: 97) |
| | | NO: 92) | | |
| 76 | TB640 057 | FTERDYAMT | TITGSGGNTYYADAVKG | KDRLSITIRPRYYGLDV |
| | | (SEQ ID | (SEQ ID NO: 95) | (SEQ ID NO: 94) |
| | | NO: 92) | | |
| 78 | TB640 109 | FTERDYAMT | LISGSGGKTYYADAVKG | VDRLTKTIRPQYYGLSV |
| | | (SEQ ID | (SEQ ID NO: 83) | (SEQ ID NO: 98) |
| | | NO: 92) | | |

LCDRs of top VL variants are shown in Table 25.

TABLE 21
LCDR sequences of top TB640_005 variants

| SEQ | | LCDR1 | LCDR2 | LCDR3 |
|---|---|---|---|---|
| ID | | 2 3 | 5 6 | 9 0 |
| NO: | Clone ID | 4 9 | 5 1 | 4 2 |
| 29 | FJ2246MP10A09 | RSSQSLLYSIGYNYLD | LGSKRAS | MQGLHSPLS |
| | | (SEQ ID NO: 88) | (SEQ ID NO: 90) | (SEQ ID NO: 338) |
| 31 | FJ2246MP10F04 | RSSKSLLYSIGYNYLD | LGSKRAS | MQGLHSPLT |
| | | (SEQ ID NO: 263) | (SEQ ID NO: 90) | (SEQ ID NO: 339) |
| 32 | FJ2246MP10B12 | RSSQSLLYSIGYNYLD | LGSKRAS | MQGLHSPLS |
| | | (SEQ ID NO: 88) | (SEQ ID NO: 90) | (SEQ ID NO: 338) |
| 35 | FJ2246MP10H11 | RSSQSLLYSIGYNYLD | LGSKRAS | MQGLHSPLS |
| | | (SEQ ID NO: 88) | (SEQ ID NO: 90) | (SEQ ID NO: 338) |
| 43 | FJ2246MP10G07 | RSSQSLLYSIGYNYLD | LGSKRAS | MQGLHSPLT |
| | | (SEQ ID NO: 88) | (SEQ ID NO: 90) | (SEQ ID NO: 339) |
| 56 | FJ2246MP10B07 | RSSQSLLYSIGYNYLD | LGSKRAS | MQGLHSPLT |
| | | (SEQ ID NO: 88) | (SEQ ID NO: 90) | (SEQ ID NO: 339) |
| 60 | FJ2246MP10H03 | RSSQSLLYSIGYNYLD | LGSKRAS | MQGLRSPLS |
| | | (SEQ ID NO: 88) | (SEQ ID NO: 90) | (SEQ ID NO: 345) |
| 61 | FJ2246MP10A10 | RSSQSLLYSIGYNYLD | LGSKRAS | MQGLHSPLT |
| | | (SEQ ID NO: 88) | (SEQ ID NO: 90) | (SEQ ID NO: 339) |
| 62 | FJ2246MP10B02 | RSSQSLLYSIGYNYLD | LGSKRAS | MQGLHSPLT |
| | | (SEQ ID NO: 88) | (SEQ ID NO: 90) | (SEQ ID NO: 339) |
| 68 | FJ2246MP10G04 | RSSQSLLYSIGYNYLD | LGSKRAS | MQGLQSPLS |
| | | (SEQ ID NO: 88) | (SEQ ID NO: 90) | (SEQ ID NO: 141) |
| 70 | FJ2246MP10D01 | RSSQSLLYSIGYNYLD | LGSKRAS | MQGLQSPLS |
| | | (SEQ ID NO: 88) | (SEQ ID NO: 90) | (SEQ ID NO: 141) |
| 71 | FJ2246MP10E12 | RSSQSLLYSIGYNYLD | LGSKRAS | MQGLQSPLS |
| | | (SEQ ID NO: 88) | (SEQ ID NO: 90) | (SEQ ID NO: 141) |
| 77 | TB640 057 | RADQSLLYSIGYNYLD | LGSKRAS | MQAIQAPYT |
| | | (SEQ ID NO: 105) | (SEQ ID NO: 90) | (SEQ ID NO: 102) |
| 79 | TB640 109 | RADQSLLYSIGYNYLD | LGSKRAS | MQALRDPYT |
| | | (SEQ ID NO: 105) | (SEQ ID NO: 90) | (SEQ ID NO: 103) |

### Example 5: Generation and Characterization of Affinity Matured Anti-Canine IL-4RA Antibodies

Based on the results of the two affinity maturation strategies, a series of heavy and light chain sequences were synthesized (SEQ ID NOS:11-22) and subcloned into pcDNA3.4. Both affinity maturation strategies identified the variant S52N in HCDR2 as a variant that improved the affinity but this variant was not included in the constructs since this introduced a potential deamidation site (NG) which is a significant sequence liability. Table 26 below has the CDR sequences of the different heavy chains and the underlined and bolded residues are those that differ from TB640_275 heavy chain. The CDR sequences as shown include Kabat and IMGT CDR positions. The frameworks of the heavy chains are IGHV3-9*01.

TABLE 22
HCDR sequences of the different variant heavy chains

| SEQ | | | | |
|---|---|---|---|---|
| ID | | | | |
| NO | Clone | HCDR1 | HCDR2 | HCDR3 |
| 11 | FJP07H09-H | GFTFRDYAMD | LISGSGGKTYYADAVKG | AVDRLTKTIRPQYYGLWS |
| | | (SEQ ID NO: 358) | (SEQ ID NO: 83) | (SEQ ID NO: 361) |
| 12 | FJP06D01-H | GFPFRDYAMD | LISGSGGKTYYADAVKG | AVDRLTKTIRPQYYGLWV |
| | | (SEQ ID NO: 359) | (SEQ ID NO: 83) | (SEQ ID NO: 87) |
| 13 | FJP05A03-H | GLPFRDYAMA | LISGSGGKTYYADAVKG | AVDRLTKTIRPQYYGLWV |
| | | (SEQ ID NO: 360) | (SEQ ID NO: 83) | (SEQ ID NO: 87) |
| 14 | Fjmix_PS-H | GFPFRDYAMD | LISGSGGKTYYADAVKG | AVDRLTKTIRPQYYGLWS |
| | | (SEQ ID NO: 359) | (SEQ ID NO: 83) | (SEQ ID NO: 361) |
| 15 | Gfmix_PTH-H | GFPFRDYAMT | LISGSGGKTYYADAVKG | AVDRLTKTIRPQYYGLWH |
| | | (SEQ ID NO: 107) | (SEQ ID NO: 83) | (SEQ ID NO: 362) |
| 16 | 21188-H | GFTFRDYAMT | LISGSGGKTYYADAVKG | AVDRLTKTIRPQYYGLWH |
| | | (SEQ ID NO: 104) | (SEQ ID NO: 83) | (SEQ ID NO: 362) |
| 9 | TB640_275-H | GFTFRDYAMD | LISGSGGKTYYADAVKG | AVDRLTKTIRPQYYGLWV |
| | | (SEQ ID NO: 358) | (SEQ ID NO: 83) | (SEQ ID NO: 87) |

Table 27 below has the CDR sequences of the different light chains and the underlined and bolded residues are those that differ from TB640_275 light chain. The CDR sequences as shown include Kabat and IMGT positions. The frameworks of the light chains are IGKV12-2*01.

TABLE 23
LCDR sequences of the different variant light chains.

| SEQ | | | | |
|---|---|---|---|---|
| ID | | | | |
| NO: | Clone | LCDR1 | LCDR2 | LCDR3 |
| 17 | FJP10A09-L | RSSQSLTYSIGYNYLD | LGSKRAS | MQGLHSPLS |
| | | (SEQ ID NO: 363) | (SEQ ID | (SEQ ID |
| | | | NO: 90) | NO: 338) |
| 18 | FJP10F04-L | RSSKSLLYSIGYNYLD | LGSKRAS | MQGLHSPLT |
| | | (SEQ ID NO: 263) | (SEQ ID | (SEQ ID |
| | | | NO: 90) | NO: 339) |
| 19 | GFmix THS-L | RSSQSLTYSIGYNYLD | LGSKRAS | MQGLHSPLG |
| | | (SEQ ID NO: 363) | (SEQ ID | (SEQ ID |
| | | | NO: 90) | NO: 365) |
| 20 | GFmix2_KTHG-L | RSSKSLTYSIGYNYLD | LGSKRAS | MQGLHSPLG |
| | | (SEQ ID NO: 364) | (SEQ ID | (SEQ ID |
| | | | NO: 90) | NO: 365) |
| 21 | GFmix3_KTHS-L | RSSKSLTYSIGYNYLD | LGSKRAS | MQGLHSPLS |
| | | (SEQ ID NO: 364) | (SEQ ID | (SEQ ID |
| | | | NO: 90) | NO: 338) |
| 22 | 21188-L | RSSQSLTYSIGYNYLD | LGSKRAS | MQGLQSPLG |
| | | (SEQ ID NO: 363) | (SEQ ID | (SEQ ID |
| | | | NO: 90) | NO: 139) |
| 10 | TB640_275-L | RSSQSLLYSIGYNYLD | LGSKRAS | MQGLQSPLT |
| | | (SEQ ID NO: 88) | (SEQ ID | (SEQ ID |
| | | | NO: 90) | NO: 101) |

The variant heavy chain constructs were co-transfected with the 21188 light chain construct in a 1:1 ratio in CHO cells and purified using Protein A resin. In a separate experiment, the variant heavy chain 21188 was co-transfected with the variant light chain constructs. The expression and purification were the same as described above.

Both the heavy and light chain variants were evaluated for their binding kinetics to canine IL-4RA using SPR on a Biacore T200. The antibodies were captured using a CM5 Series S chip amine coupled with an anti-dog Fc antibody (Jackson Immunoresearch). Antigen, canine IL4Ra (SEQ ID NO:2), binding was then assessed at multiple concentrations starting at 100 nM using PBSP+ running buffer (Cytiva) with a flow rate of 30 uL/min. The length of the association time was 120 s and the dissociation time was run for 600 s. The chip surface was regenerated with 10 mM glycine. Double reference-subtracted sensorgrams were fitted to a 1:1 binding model using Biacore T200 Evaluation software. The binding kinetics of the heavy chain variants when paired with 21188 light chain are shown in Table 28 below.

TABLE 24
Binding kinetics of heavy chain variants when paired
with 21188 light chain.

| | Light | | | | Rmax |
|---|---|---|---|---|---|
| Heavy Chain | Chain | ka (1/Ms) | kd (1/s) | KD (M) | (RU) |
| FJP07H09-H | 21188-L | 2.36E+05 | 2.53E−04 | 1.07E−09 | 83.3 |
| FJP06D01-H | 21188-L | 2.3E+05 | 2.08E−04 | 9.08E−10 | 71.4 |
| FJP05A03-H | 21188-L | 2.79E+05 | 1.05E−04 | 3.77E−10 | 85.6 |
| Fjmix_PS-H | 21188-L | 2.35E+05 | 2.06E−04 | 8.78E−10 | 78.3 |
| Gfmix PTH-H | 21188-L | 2.55E+05 | 9.71E−05 | 3.81E−10 | 69.7 |
| 21188-H | 21188-L | 2.46E+05 | 9.85E−05 | 4E−10 | 74.4 |

The binding kinetics of the light chain variants when paired with 21188 heavy chain are shown in Table 29.

TABLE 25
Binding kinetics of light chain variants when paired
with 21188 heavy chain.

| Heavy | | | | | Rmax |
|---|---|---|---|---|---|
| Chain | Light Chain | ka (1/Ms) | kd (1/s) | KD (M) | (RU) |
| 21188-H | FJP10A09-L | 3.24E+05 | 4.62E−05 | 1.43E−10 | 71.5 |
| 21188-H | FJP10F04-L | 2.86E+05 | 6.81E−05 | 2.38E−10 | 74.3 |
| 21188-H | GFmix_THS-L | 3.57E+05 | 5.78E−05 | 1.62E−10 | 72.6 |
| 21188-H | GFmix2_KTHG-L | 3.54E+05 | 5.92E−05 | 1.68E−10 | 89.0 |
| 21188-H | GFmix3_KTHS-L | 3.33E+05 | 4.79E−05 | 1.44E−10 | 82.9 |
| 21188-H | 21188-L | 3.32E+05 | 5.96E−05 | 1.8E−10 | 70.3 |

Six different combinations of heavy and light chain variants (Table 30) were co-transfected and produced as described above. The binding kinetics of the heavy and light chain variants to canine IL-4RA was determined using the SPR methods described above. The results of the binding kinetics are shown below in Table 30.

TABLE 26
Binding kinetics of heavy and light chain combinations

| | | ka | | | Rmax |
|---|---|---|---|---|---|
| Heavy Chain | Light Chain | (1/Ms) | kd (1/s) | KD (M) | (RU) |
| FJP05A03-H | GFmix_THS-L | 4.92E+05 | 8.32E−05 | 1.69E−10 | 87.9 |
| FJP05A03-H | FJP10A09-L | 4.42E+05 | 8.87E−05 | 2.01E−10 | 83.2 |
| FJP05A03-H | GFmix3_ | 4.66E+05 | 8.68E−05 | 1.87E−10 | 90.7 |
| | KTHS-L | | | | |
| GFmix PTH-H | GFmix_THS-L | 4.58E+05 | 7.78E−05 | 1.71E−10 | 84.8 |
| GFmix PTH-H | FJP10A09-L | 4.19E+05 | 7.00E−05 | 1.67E−10 | 81.8 |
| GFmix PTH-H | GFmix3_ | 4.47E+05 | 7.58E−05 | 1.70E−10 | 94.1 |
| | KTHS-L | | | | |
| 21188-H | 21188-L | 4.10E+05 | 9.93E−05 | 2.43E−10 | 101.3 |
| TB640_005-H | TB640_005-L | 2.12E+05 | 4.86E−04 | 2.29E−09 | 67.8 |

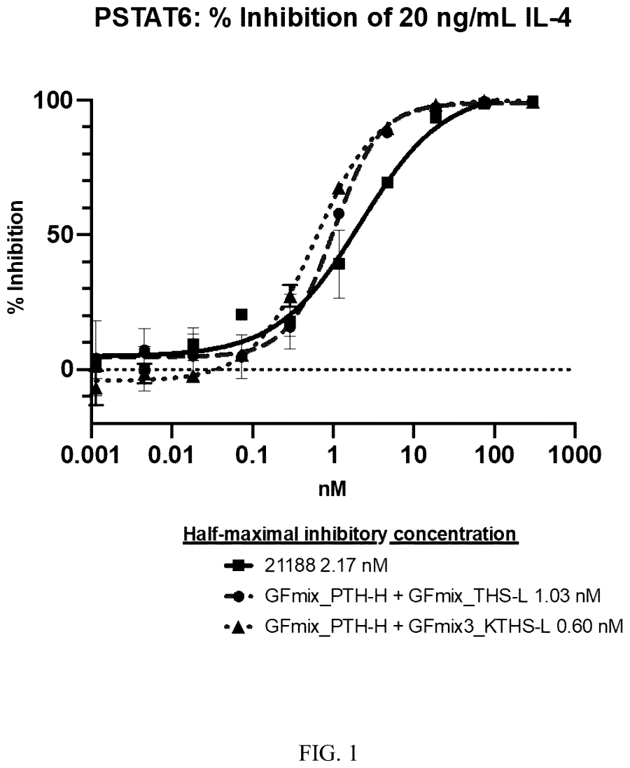

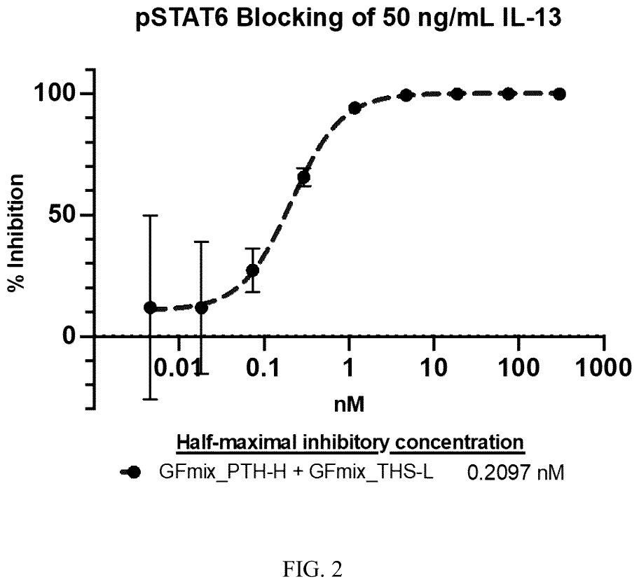

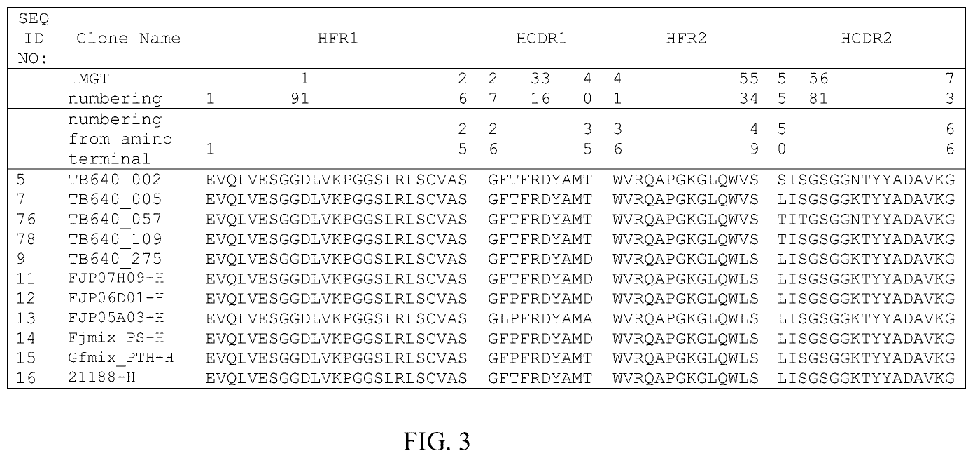

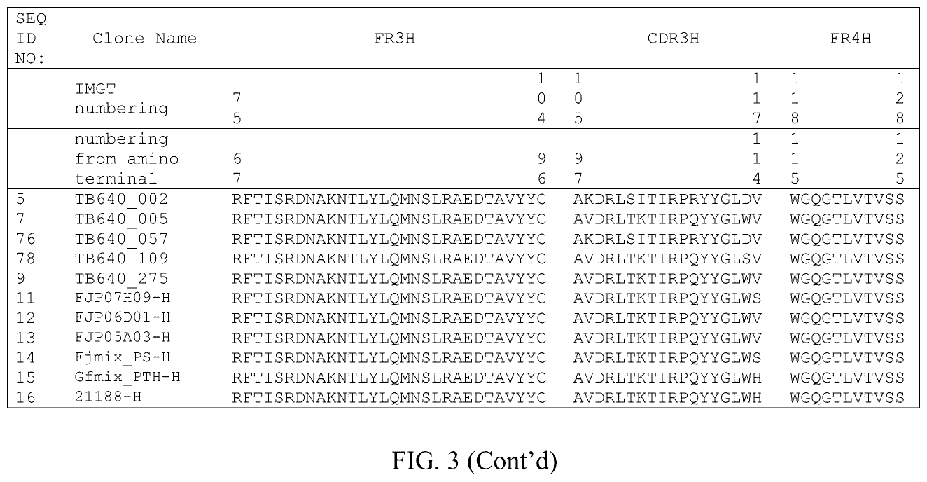

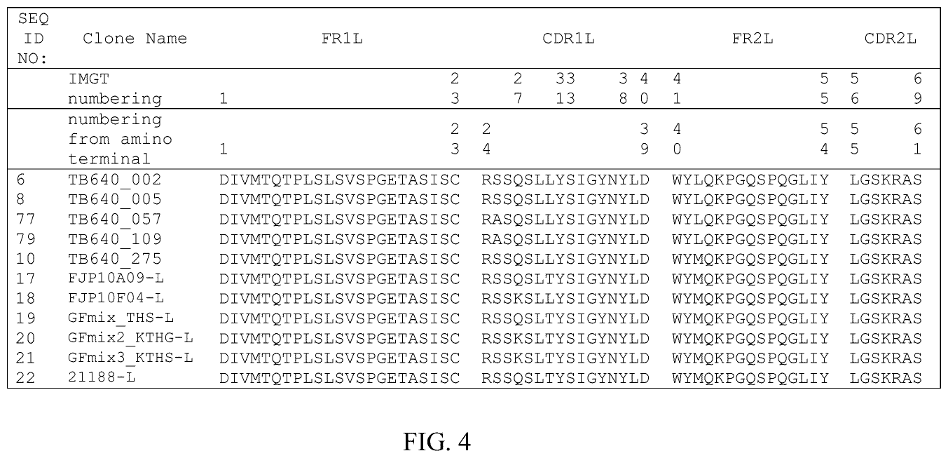

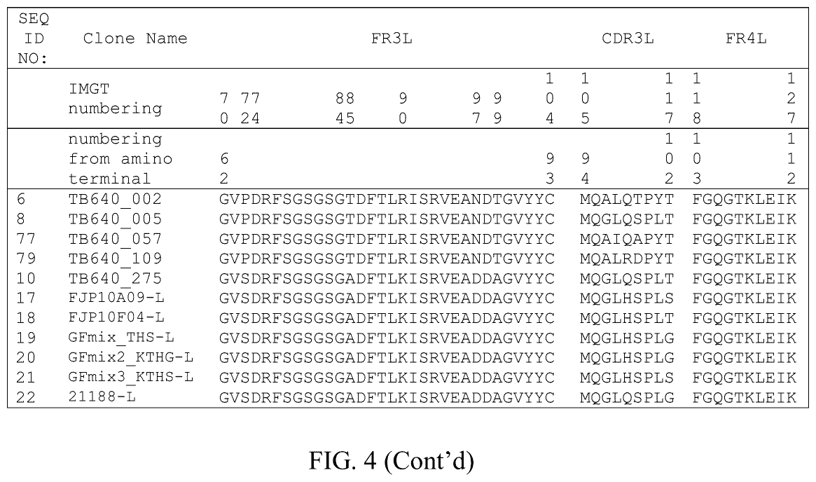

Three antibodies (GFmix_PTH-H+GFmix_THS-L; GFmix_PTH-H+GFmix3_KTHS-L; 21188-H+21188-L) were tested in a functional assay with the canine DH82 cells (FIGS. 1 and 2). In this assay, the antibodies were tested for their ability to block either canine JL-4 or canine JL-13 signaling through the canine JL-4RA. The canine JL-4RA signaling was measured using the phospo-STAT6 assay which measures the tyrosine phosphorylation of STAT6. In a 96 well TC-Spectra plate, 80,000 cells were seeded per well and incubated at 37 CC and 5% CO₂ overnight. The next day, cells were treated with serially diluted antibodies as indicated for 15 minutes followed by 20 ng/mL canine JL-4 (R&D Systems) or 50 ng/mL canine JL-13 (R&D Systems) for 15 minutes. The AlphaLISA pSTAT6 protocol (Revvity) was followed to harvest cells and detect pTyr641 in lysis. Resulting luminescence was read on a ThermoFisher Varoskian Lux. The results demonstrate that all three antibodies block canine IL-4 signaling and the antibody GFmix_PTH-H+GFmix_THS-L blocks canine IL-13 signaling.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An antigen binding protein that specifically binds to interleukin-4 receptor alpha (IL-4RA), which comprises:
(a) a heavy chain complementarity determining region 1 (HCDR1) comprising X₂₇X₂₈X₂₉X₃₀DX₃₂AMX₃₅, wherein X₂₇ comprises F, I, L, or V, X₂₈ comprises A, D, I, N, P, R, S, or T, X₂₉ comprises F or S, X₃₀ comprises D, G, N, R, or S, X₃₂ comprises F or Y, and X₃₅ comprises A, D, S, T, or V;
(b) a heavy chain complementarity determining region 2 (HCDR2) comprising X₅₀X₅₁X₅₂GX₅₄X₅₅X₅₆X₅₇X₅₈YYADAVKX₆₆, wherein X₅₀ comprises F, L, M, S, or T, X₅₁ comprises I or V, X₅₂ comprises N, S, or T, X₅₄ comprises D, I, N, S, or T, X₅₅ comprises A, G, or S, X₅₆ comprises D, G, N, S, or T, X₅₇ comprises G, K, N, R, or S, X₅₈ comprises I, S, T, or V, and X₆₆ comprises D or G;
(c) a heavy chain complementarity determining region 3 (HCDR3) comprising X₉₈DRX₁₀₁X₁₀₂X₁₀₃X₁₀₄X₁₀₅RPX₁₀₈YX₁₁₀GX₁₁₂X₁₁₃X₁₁₄, wherein X₉₈ comprises A, I, K, R, S, T, or V, X₁₀₁ comprises I, K, L, or M, X₁₀₂ comprises S or T, X₁₀₃ comprises I, H, N, K, or R, X₁₀₄ comprises M or T, X₁₀₅ comprises I or V, X₁₀₈ comprises A, Q, R, or V, X₁₁₀ comprises A, E, F, L, M, N, R, S, T, Q, or Y, X₁₁₂ comprises F, L, or M, X₁₁₃ comprises D, Q, S, or W, and X₁₁₄ comprises, A, D, F, G, H, K, L, Q, R, S, or V;
(d) a light chain complementarity determining region 1 (LCDR1) comprising X₂₇X₂₈X₂₉X₃₀YX₃₂X₃₃X₃₄X₃₅NYX₃₈D, wherein X₂₇ comprises E, G, H, K, L, Q, R, or T, X₂₈ comprises S or T, X₂₉ comprises L or V, X₃₀ comprises A, I, L, Q, S, or T, X₃₂ comprises G or S, X₃₃ comprises A, G, I, N, P, S, T, or V, X₃₄ comprises D, G, L, or R, X₃₅ comprises A, F, I, K, L, M, N, R, S, T, or Y, and X₃₈ comprises I, L, or V;
(e) a light chain complementarity determining region 2 (LCDR2) comprising X₅₅X₅₆X₅₇X₅₈X₅₉X₆₀X₆₁, wherein X₅₅ comprises A, H, L, M, N, or Q, X₅₆ comprises G or S, X₅₇ comprises D, H, N, S, T, or Y, X₅₈ comprises K, N, or R, X₅₉ comprises L or R, X₆₀ comprises A, M, Q, S, or Y, and X₆₁ comprises A, G, P, Q, R, S, or T; and
(f) a light chain complementarity determining region 3 (LCDR3) comprising MX₉₅X₉₆X₉₇X₉₈X₉₉PX₁₀₁X₁₀₂, wherein X₉₅ comprises N, or Q, X₉₆ comprises A, G, N, P, or S, X₉₇ comprises I or L, X₉₈ comprises F, H, I, Q, R, S, or T, X₉₉ comprises A, D, F, N, S, or T, X₁₀₁ comprises I, L, N, P, V, or Y, and X₁₀₂ comprises G, L, N, S, T, or V.

2. The antigen binding protein of claim 1, wherein:
HCDR1 comprises FX₂₈FRDYAMT, wherein X₂₈ comprises N, P, or T;
HCDR2 comprises X₅₀IX₅₂GSGGX₅₇TYYADAVKG, wherein X₅₀ comprises F, L, M, S, or T, X₅₂ comprises N, or S, and X₅₇ comprises K, or N;
HCDR3 comprises X₉₈DRX₁₀₁X₁₀₂X₁₀₃TIRPX₁₀₈YX₁₁₀GX₁₁₂X₁₁₃X₁₁₄, wherein X₉₈ comprises K, or V, X₁₀₁ comprises K, L, or M, X₁₀₂ comprises S or T, X₁₀₃ comprises I, K, or R, X₁₀₈ comprises R, or Q, X₁₁₀ comprises L, R, or Y, X₁₁₂ comprises L, or M, X₁₁₃ comprises D, S, or W, and X₁₁₄ comprises G, H, K, Q, or V; and/or
LCDR1 comprises X₂₇SLX₃₀YSX₃₃GX₃₅NYLD, wherein X₂₇ comprises E, H, L, Q, or T, X₃₀ comprises A, L, or T, X₃₃ comprises A, I, P, or V, and X₃₅ comprises M, or Y;
LCDR2 comprises X₅₅GX₅₇KRX₆₀S, wherein X₅₅ comprises L, of M, X₅₇ comprises H, N, S, or T, and X₆₀ comprises A, L, or M; and
LCDR3 comprises MX₉₅X₉₆X₉₇X₉₈X₉₉PX₁₀₁X₁₀₂, wherein X₉₅ comprises N, or Q, X₉₆ comprises A, G, or P, X₉₇ comprises I or L, X₉₈ comprises Q, or R, X₉₉ comprises A, D, S, or T, X₁₀₀ comprises P, X₁₀₁ comprises L, N, or Y, and X₁₀₂ comprises G, L, N, S, or T.

3. The antigen binding protein of claim 2, wherein:
HCDR1 comprises SEQ ID NO:104, or SEQ ID NO:106, or SEQ ID NO: 107;
HCDR2 comprises SEQ ID NO:83, or SEQ ID NO:108, or SEQ ID NO: 109, or SEQ ID NO:110;
HCDR3 comprises SEQ ID NO:86, or SEQ ID NO:111, or SEQ ID NO:112, or SEQ ID NO:113, or SEQ ID NO:114, or SEQ ID NO:115, or SEQ ID NO:116, or SEQ ID NO:117, or SEQ ID NO:118, or SEQ ID NO:119, or SEQ ID NO:120, or SEQ ID NO:121;
LCDR1 comprises SEQ ID NO:88, or SEQ ID NO:122, or SEQ ID NO:123, or SEQ ID NO:124, or SEQ ID NO:125, or SEQ ID NO:126, or SEQ ID NO:127, or SEQ ID NO:128, or SEQ ID NO:129, or SEQ ID NO:130, or SEQ ID NO:131, or SEQ ID NO:132;
LCDR2 comprises SEQ ID NO:90, or SEQ ID NO:133, or SEQ ID NO:134, or SEQ ID NO:135, or SEQ ID NO:136, or SEQ ID NO:137; and/or
LCDR3 comprises SEQ ID NO:101, or SEQ ID NO:138, or SEQ ID NO:139, or SEQ ID NO:140, or SEQ ID NO:141, or SEQ ID NO:142, or SEQ ID NO:143, or SEQ ID NO:144.

4. The antigen binding protein of claim 1, wherein:
HCDR1 comprises FTFRDYAMT; and/or
HCDR2 comprises LIX₅₂GSGGKTYYADAVKG, wherein X₅₂ comprises N, or S; and/or
HCDR3 comprises DRX₁₀₁TKTIRPQYYGLWX₁₁₄, wherein X₁₀₁ comprises K or L, and X₁₁₄ comprises H or V; and/or
LCDR1 comprises QSLX₃₀YSIGYNYLD, wherein X₃₀ comprises L, or T; and/or
LCDR2 comprises LGX₅₇KRAS, wherein X₅₇ comprises H or S; and/or
LCDR3 comprises MQX₉₆LGQPLX₁₀₂, wherein X₉₆ comprises G or P, and X₁₀₂ comprises G, L, N, S, or T.

5. The antigen binding protein of claim 4, which comprises a heavy chain variable domain at least 80%, at least 85%, at least 90%, at least 95%, at least 97%, or 100% identical to a heavy chain variable domain of SEQ ID NO:5, SEQ ID NO:7, SEQ ID NO:15, SEQ ID NO:16, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:30, SEQ ID NO:33, SEQ ID NO:34, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:57, SEQ ID NO:58, SEQ ID NO:59, SEQ ID NO:63, SEQ ID NO:64, SEQ ID NO:65, SEQ ID NO:66, SEQ ID NO:67, SEQ ID NO:68, SEQ ID NO:69, SEQ ID NO:72, SEQ ID NO:73, SEQ ID NO:74, SEQ ID NO:75, SEQ ID NO:76, or SEQ ID NO:78, and a light chain variable domain at least 80%, at least 85%, at least 90%, at least 95%, at least 97%, or 100% identical to a light chain variable domain of SEQ ID NO:6, SEQ ID NO:8, SEQ ID NO:17, SEQ ID NO:18, SEQ ID NO:19, SEQ ID NO:20, SEQ ID NO:21, SEQ ID NO:22, SEQ ID NO:29, SEQ ID NO:31, SEQ ID NO:32, SEQ ID NO:35, SEQ ID NO:43, SEQ ID NO:56, SEQ ID NO:60, SEQ ID NO:61, SEQ ID NO:62, SEQ ID NO:68, SEQ ID NO:70, SEQ ID NO:71, SEQ ID NO:77, or SEQ ID NO:79.

6. The antigen binding protein of claim 1, wherein:
HCDR1 comprises X₂₇X₂₈FRDX₃₂AMX₃₅, wherein X₂₇ comprises F or L, X₂₈ comprises P or T, X₃₂ comprises F or Y, and X₃₅ comprises A or T;
HCDR2 comprises X₅₀X₅₁X₅₂GX₅₄X₅₅X₅₆X₅₇X₅₈YYADAVKG, wherein X₅₀ comprises L or T, X₅₁ comprises I or V, X₅₂ comprises N, S, or T, X₅₄ comprises D, N, S, or T, X₅₅ comprises A, G, or S, X₅₆ comprises G, S, or T, X₅₇ comprises K or N, and X₅₈ comprises T or V;
HCDR3 comprises X₉₈DRLX₁₀₂X₁₀₃TX₁₀₅RPX₁₀₈YYGX₁₁₂X₁₁₃X₁₁₄, wherein X₉₈ comprises K or V, X₁₀₂ comprises S or T, X₁₀₃ comprises I or K, X₁₀₅ comprises I or V, X₁₀₈ comprises Q or R, X₁₁₂ comprises L or M, X₁₁₃ comprises D, S, or W, and X₁₁₄ comprises A, S, or V;
LCDR1 comprises RX₂₅X₂₆X₂₇SLLYSIGYNYLD, wherein X₂₅ comprises A or S, X₂₆ comprises D or S, and X₂₇ comprises K or Q;
LCDR2 comprises LGSKARAS; and
LCDR3 comprises MQX₉₆X₉₇X₉₈X₉₉PX₁₀₁X₁₀₂, wherein X₉₆ comprises A or G, X₉₇ comprises I or L, X₉₈ comprises H, Q, or R, X₉₉ comprises A, D, or S, X₁₀₁ comprises L or Y, and X₁₀₂ comprises S or T.

7. The antigen binding protein of claim 6, wherein:
HCDR1 comprises SEQ ID NO:92, HCDR2 comprises SEQ ID NO:108, and HCDR3 comprises SEQ ID NO:248; or
HCDR1 comprises SEQ ID NO:92, HCDR2 comprises SEQ ID NO:108, and HCDR3 comprises SEQ ID NO:250; or
HCDR1 comprises SEQ ID NO:92, HCDR2 comprises SEQ ID NO:212, and HCDR3 comprises SEQ ID NO:244; or
HCDR1 comprises SEQ ID NO:158, HCDR2 comprises SEQ ID NO:216, and HCDR3 comprises SEQ ID NO:97; or
HCDR1 comprises SEQ ID NO:92, HCDR2 comprises SEQ ID NO:108, and HCDR3 comprises SEQ ID NO:252; or
HCDR1 comprises SEQ ID NO:158, HCDR2 comprises SEQ ID NO:108, and HCDR3 comprises SEQ ID NO:97; or
HCDR1 comprises SEQ ID NO:92, HCDR2 comprises SEQ ID NO:202, and HCDR3 comprises SEQ ID NO:97; or
HCDR1 comprises SEQ ID NO:92, HCDR2 comprises SEQ ID NO:200, and HCDR3 comprises SEQ ID NO:97; or
HCDR1 comprises SEQ ID NO:92, HCDR2 comprises SEQ ID NO:108, and HCDR3 comprises SEQ ID NO:97; or
HCDR1 comprises SEQ ID NO:92, HCDR2 comprises SEQ ID NO:203, and HCDR3 comprises SEQ ID NO:97; or
HCDR1 comprises SEQ ID NO:92, HCDR2 comprises SEQ ID NO:195, and HCDR3 comprises SEQ ID NO:97; or
HCDR1 comprises SEQ ID NO:92, HCDR2 comprises SEQ ID NO:211, and HCDR3 comprises SEQ ID NO:97; or
HCDR1 comprises SEQ ID NO:185, HCDR2 comprises SEQ ID NO:83, and HCDR3 comprises SEQ ID NO:97; or
HCDR1 comprises SEQ ID NO:92, HCDR2 comprises SEQ ID NO:197, and HCDR3 comprises SEQ ID NO:97; or
HCDR1 comprises SEQ ID NO:92, HCDR2 comprises SEQ ID NO:83, and HCDR3 comprises SEQ ID NO:248; or
HCDR1 comprises SEQ ID NO:156, HCDR2 comprises SEQ ID NO:83, and HCDR3 comprises SEQ ID NO:97; or
HCDR1 comprises SEQ ID NO:186, HCDR2 comprises SEQ ID NO:83, and HCDR3 comprises SEQ ID NO:97; or
HCDR1 comprises SEQ ID NO:92, HCDR2 comprises SEQ ID NO:196, and HCDR3 comprises SEQ ID NO:97; or
HCDR1 comprises SEQ ID NO:92, HCDR2 comprises SEQ ID NO:95, and HCDR3 comprises SEQ ID NO:94; or
HCDR1 comprises SEQ ID NO:92, HCDR2 comprises SEQ ID NO:83, and HCDR3 comprises SEQ ID NO:98;
and/or wherein LCDR1 comprises SEQ ID NO:88, LCDR2 comprises SEQ ID NO:90, and LCDR3 comprises SEQ ID NO:338; or
LCDR1 comprises SEQ ID NO:263, LCDR2 comprises SEQ ID NO:90, and LCDR3 comprises SEQ ID NO:339; or
LCDR1 comprises SEQ ID NO:88, LCDR2 comprises SEQ ID NO:90, and LCDR3 comprises SEQ ID NO:339; or
LCDR1 comprises SEQ ID NO:88, LCDR2 comprises SEQ ID NO:90, and LCDR3 comprises SEQ ID NO:345; or
LCDR1 comprises SEQ ID NO:88, LCDR2 comprises SEQ ID NO:90, and LCDR3 comprises SEQ ID NO:141; or
LCDR1 comprises SEQ ID NO:105, LCDR2 comprises SEQ ID NO:90, and LCDR3 comprises SEQ ID NO:102; or
LCDR1 comprises SEQ ID NO:105, LCDR2 comprises SEQ ID NO:90, and LCDR3 comprises SEQ ID NO:103.

8. The antigen binding protein of claim 7, which comprises a heavy chain variable domain at least 80%, at least 85%, at least 90%, at least 95%, at least 97%, or 100% identical to SEQ ID NO:23, SEQ ID NO:24, SEQ ID NO:25, SEQ ID NO:26, SEQ ID NO:27, SEQ ID NO:28, SEQ ID NO:30, SEQ ID NO:33, SEQ ID NO:34, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:57, SEQ ID NO:58, SEQ ID NO:59, SEQ ID NO:63, SEQ ID NO:64, SEQ ID NO:65, SEQ ID NO:66, SEQ ID NO:67, SEQ ID NO:69, SEQ ID NO:72, SEQ ID NO:73, SEQ ID NO:74, SEQ ID NO:75, SEQ ID NO:76, or SEQ ID NO:78 and/or a light chain variable domain at least 80%, at least 85%, at least 90%, at least 95%, at least 97%, or 100% identical to SEQ ID NO:29, SEQ ID NO:31, SEQ ID NO:32, SEQ ID NO:35, SEQ ID NO:43, SEQ ID NO:56, SEQ ID NO:60, SEQ ID NO:61, SEQ ID NO:62, SEQ ID NO:68, SEQ ID NO:70, SEQ ID NO:71, SEQ ID NO:77, or SEQ ID NO:79.

9. The antigen binding protein of claim 1, wherein HCDR1 comprises SEQ ID NO:358, HCDR2 comprises SEQ ID NO:83, and HCDR3 comprises SEQ ID NO:361, or HCDR1 comprises SEQ ID NO:359, HCDR2 comprises SEQ ID NO:83, and HCDR3 comprises SEQ ID NO:87, or HCDR1 comprises SEQ ID NO:360, HCDR2 comprises SEQ ID NO:83, and HCDR3 comprises SEQ ID NO:87, or HCDR1 comprises SEQ ID NO:359, HCDR2 comprises SEQ ID NO:83, and HCDR3 comprises SEQ ID NO:361, or HCDR1 comprises SEQ ID NO:107, HCDR2 comprises SEQ ID NO:83, and HCDR3 comprises SEQ ID NO:362, or HCDR1 comprises SEQ ID NO:104, HCDR2 comprises SEQ ID NO:83, and HCDR3 comprises SEQ ID NO:362, or HCDR1 comprises SEQ ID NO:358, HCDR2 comprises SEQ ID NO:83, and HCDR3 comprises SEQ ID NO:87, and/or wherein LCDR1 comprises SEQ ID NO:363, LCDR2 comprises SEQ ID NO:90, and LCDR3 comprises SEQ ID NO:338, or LCDR1 comprises SEQ ID NO:263, LCDR2 comprises SEQ ID NO:90, and LCDR3 comprises SEQ ID NO:339, or LCDR1 comprises SEQ ID NO:363, LCDR2 comprises SEQ ID NO:90, and LCDR3 comprises SEQ ID NO:365, or LCDR1 comprises SEQ ID NO:364, LCDR2 comprises SEQ ID NO:90, and LCDR3 comprises SEQ ID NO:338, or LCDR1 comprises SEQ ID NO:363, LCDR2 comprises SEQ ID NO:90, and LCDR3 comprises SEQ ID NO:139, or wherein LCDR1 comprises SEQ ID NO:88, LCDR2 comprises SEQ ID NO:90, and LCDR3 comprises SEQ ID NO:101.

10. The antigen binding protein of claim 9, which comprises a heavy chain variable domain at least 80%, at least 85%, at least 90%, at least 95%, at least 97%, or 100% identical to SEQ ID NO: 11, SEQ ID NO:12, SEQ ID NO:13, SEQ ID NO:14, SEQ ID NO:15, or SEQ ID NO:16 and a light chain variable domain at least 80%, at least 85%, at least 90%, at least 95%, at least 97%, or 100% identical to SEQ ID NO:17, SEQ ID NO:18, SEQ ID NO:19, SEQ ID NO:20, SEQ ID NO:21, or SEQ ID NO:22.

11. The antigen binding protein of claim 9, wherein the heavy chain variable domain comprises SEQ ID NO:13 and the light chain variable domain comprises SEQ ID NO:19, or the heavy chain variable domain comprises SEQ ID NO:13 and the light chain variable domain comprises SEQ ID NO:17, or the heavy chain variable domain comprises SEQ ID NO:13 and the light chain variable domain comprises SEQ ID NO:21, or the heavy chain variable domain comprises SEQ ID NO:15 and the light chain variable domain comprises SEQ ID NO:19, or the heavy chain variable domain comprises SEQ ID NO:15 and the light chain variable domain comprises SEQ ID NO:17, or the heavy chain variable domain comprises SEQ ID NO:15 and the light chain variable domain comprises SEQ ID NO:21, or the heavy chain variable domain comprises SEQ ID NO:16 and the light chain variable domain comprises SEQ ID NO:12.

12. The antigen binding protein of claim 1, which comprises no more than two (2) changes in any VH-CDR as compared to SEQ ID NO:15 and no more than two (2) changes in any VL-CDR as compared to SEQ ID NO:19.

13. The antigen binding protein of claim 1, which comprises no more than one (1) change in any VH-CDR as compared to SEQ ID NO:15 and no more than one (1) change in any VL-CDR as compared to SEQ ID NO:19.

14. The protein of claim 1, which comprises a heavy chain variable domain framework (HFR1+HFR2+HFR3+HFR4) at least 75%, or at least 80%, or at least 85%, or at least 90%, or at least 93%, or at least 95% identical, or identical to the heavy chain variable domain framework of SEQ ID NO:15.

15. The protein of claim 1, which comprises a light chain variable domain framework (LFR1+LFR2+LFR3+LFR4) at least 75%, or at least 80%, or at least 85%, or at least 90%, or at least 93%, or at least 95% identical, or identical to the light chain variable domain framework of SEQ ID NO:19.

16. An isolated nucleic acid sequence encoding an anti-IL-4RA antigen binding protein of claim 1.

17. A vector that comprises the nucleic acid of claim 16.

18. A recombinant cell which comprises the nucleic acid of claim 16.

19. A cell that expresses the antigen binding protein of claim 1.

20. A method of producing an antigen binding protein that specifically binds to interleukin-4 receptor alpha (IL-4RA), which comprises culturing the cell of claim 19 under conditions that result in production of the antigen binding protein.

21. A pharmaceutical composition comprising a therapeutically effective amount of the anti-IL-4RA protein of claim 1.

22. A method of suppressing atopic dermatitis in a subject, which comprises administering to the subject a therapeutically effective amount of an anti-IL-4RA protein of claim 1.

23. A method of suppressing asthma in a subject which comprise administering to the subject a therapeutically effective amount of an anti-IL-4RA protein of claim 1.

24. A method of suppressing IgE production in a subject which comprise administering to the subject a therapeutically effective amount of an anti-IL-4RA protein of claim 1.

25. A method of inhibiting binding of IL-4 to anti-IL-4RA in a subject, which comprises administering to the subject a therapeutically effective amount of the anti-IL-4RA antigen binding protein of claim 1.

26. The method of claim 22, wherein the subject comprises a canine, a feline, or an equine.

27. A method of detecting anti-IL-4RA in a sample comprising incubating the sample with an anti-IL-4RA protein of claim 1 and detecting the anti-IL-4RA protein bound to IL-4 in the sample.
